(19) United States
(12) Patent Application Publication
(10) Pub. No.: US 2024/0268694 A1
(43) Pub. Date: Aug. 15, 2024

(54) Noninvasive Structural and Valvular Abnormality Detection System based on Flow Aberrations

(71) Applicant: MEDICI TECHNOLOGIES, LLC, Albuquerque, NM (US)

(72) Inventors: Mark Ries Robinson, Albuquerque, NM (US); Jasmine Sanchez, Albuquerque, NM (US); Robert K Rowe, Poulsbo, WA (US)

(21) Appl. No.: 18/618,730

(22) Filed: Mar. 27, 2024

Related U.S. Application Data
(63) Continuation-in-part of application No. PCT/US2023/062068, filed on Feb. 6, 2023.
(60) Provisional application No. 63/560,549, filed on Mar. 1, 2024.

(51) Int. Cl. A61B 5/026; A61B 5/00; A61B 5/02

(52) U.S. Cl. CPC A61B 5/0261; A61B 5/02007; A61B 5/4842

(57) ABSTRACT

Embodiments provide a reliable, convenient, noninvasive, and cost-effective determination of structural and valvular abnormalities in the left ventricular outflow tract. Embodiments obtain a noninvasive optical pulse plethysmogram from the systolic and diastolic phases of the cardiac cycle for subsequent morphologic waveform analysis to determine left ventricular outflow tract anomalies. Left ventricular outflow tract abnormalities alter the rate of increase in flow during early systole and decrease in flow during late systole. These flow variances at the aortic valve are amplified as the pulse wave moves to the periphery due to a combination of the ventricular-aortic interaction, pulse augmentation, and reflections at branching vessels and changes in diameter. The invention addresses historical limitations in the noninvasive determination of structural and valvular abnormalities in the left ventricular outflow tract by using one or more of (1) improved optical measurement systems, (2) peripheral sampling locations that maximize signal differences, (3) volitional patient maneuvers to improve the diagnostic ability of the system, and (4) pulse enhancement techniques. The resulting test system can be used to determine the presence of abnormalities and to diagnose the type of abnormality. The ability to more efficiently and effectively diagnose aortic stenosis, the most common abnormality, in the primary care clinic will result in fewer patients experiencing complications such as heart failure, heart attack, and sudden death.

### TECHNICAL FIELD

The present invention relates to the noninvasive determination of structural and valvular abnormalities in the left ventricular outflow tract by analysis of noninvasive optical pulse plethysmogram. The noninvasive system enables testing in the clinic and does not require expensive direct assessment of blood flow characteristics at the location of the abnormality.

The system enables classification of the abnormality as well as a quantitative assessment of the abnormality, such as the estimation of aortic valve area.

### BACKGROUND

Aortic Stenosis Clinical Progression and Diagnostic Limitations. Aortic stenosis impacts the flow characteristic as blood is ejected from the heart. FIG. 1 is an illustration of aortic stenosis progression, flow impact and illustrates flow compromise as the valvular area becomes restricted. The narrowing requires increased pressure within the heart to pump blood across a smaller opening. This is similar to attaching smaller and smaller nozzles to the end of a garden hose (bottom row of the figure). The narrowing of the nozzle slows the forward flow of water, creates a jet of flow with higher velocities, and results in pressure buildup within the garden hose.

Aortic stenosis (AS) is the most common type of left ventricular outflow tract obstruction in developed countries. The typical course of AS involves a long asymptomatic period—many patients with severe AS are asymptomatic. Once symptoms begin, mortality increases. Without surgery, 40% to 50% of patients with classic symptoms die within 1 year. FIG. 2, reproduced from the paper by Carabello et al., graphically illustrates the consequences of aortic stenosis with significant mortality after the development of symptoms. The table illustrates the ability of the heart to maintain cardiac output even in the presence of diminished valvular area. (Carabello, Blase A., and Walter J. Paulus. “Aortic stenosis.” The Lancet 373.9667 (2009): 956-966). Good outcomes generally result with careful follow-up and monitoring in asymptomatic individuals and urgent aortic valve replacement in symptomatic individuals. Heart valve replacement is one of the most common procedures representing approximately 25% of all procedures.

The importance of early detection is critical as a high percentage of patients with asymptomatic aortic stenosis suffer irreversible damage prior to the development of symptoms. Tastet et al. investigated the degree of damage in patients with asymptomatic aortic stenosis. The study examined 735 asymptomatic patients with moderate aortic stenosis as evaluated by Doppler echocardiography. The authors found that 88% of the patients had some form of left ventricular damage. (Tastet, Lionel, et al. “Staging cardiac damage in patients with asymptomatic aortic valve stenosis.” Journal of the American College of Cardiology 74.4 (2019): 550-563). Given the consequences of undiagnosed aortic stenosis, early detection by screening is vital to avoid irreversible disease progression and preventable death. A 60+ year-old patient complaining of dyspnea after walking up the stairs represents a complicated diagnostic pathway with the need to rule out aortic stenosis. The process should involve an echocardiogram with doppler interrogation of the aortic valve as it serves as the mainstay of diagnosis. However, the process is complicated because insurance companies are reluctant to approve the test until all other potential etiologies are evaluated. The resulting process is inefficient and can result in delays in diagnosis. Thus, the need for a simple in-clinic screening test has significant value for the family practice or internal medicine physician.

Types of Left Ventricular Outflow Tract Abnormalities. Left ventricular outflow tract (LVOT) abnormalities are conditions that affect the structure or function of the left ventricular outflow tract, which is the part of the heart that carries blood from the left ventricle (the heart's main pumping chamber) to the rest of the body. These abnormalities can range from mild to severe and significantly impact heart function and overall health.

There are several types of left ventricular outflow tract abnormalities, including:

Stenosis: This is a narrowing of the left ventricular outflow tract, which can reduce the amount of blood that is able to flow out of the left ventricle and into the body. Stenosis can be caused by a variety of factors, including scar tissue, plaque buildup, or congenital defects.

Hypertrophy: This is an abnormal thickening of the walls of the left ventricle, which can lead to an enlarged left ventricular outflow tract. Hypertrophy can be caused by high blood pressure, heart disease, or other conditions.

Aneurysm: This is a bulge or ballooning in the wall of the left ventricle, which can cause the left ventricular outflow tract to become enlarged and weakened. A variety of factors, including coronary artery disease, high blood pressure, and genetics, can cause aneurysms.

Coarctation: This is a condition in which the left ventricular outflow tract is constricted or narrowed, which can reduce blood flow to the body. A variety of factors, including congenital defects and scar tissue can cause coarctation.

Left ventricular outflow tract abnormalities are often referred to by the location of the abnormality. The three main types are (1) restrictions located at the aortic valve level (valvular), (2) in the ascending aorta (supravalvular), and (3) in the proximal portion of the left ventricular outflow tract (subvalvular). Valvular stenosis is the most common type and constitutes approximately 65 to 75% of cases, whereas subvalvular and supravalvular stenosis constitutes approximately 15% to 20% and 5% to 10% of cases, respectively (Gulino, Simona, Alessio Di Landro, and Antonino Indelicato. “Aortic stenosis: epidemiology and pathogenesis.” Percutaneous Treatment of Left Side Cardiac Valves: A Practical Guide for the Interventional Cardiologist (2018): 245-252.)

The most common form of subvalvular obstruction is hypertrophic cardiomyopathy (HCM) and is a structural disease in which the heart muscle becomes abnormally thick (hypertrophied). The thickened heart muscle can make it harder for the heart to pump blood effectively. The resulting left ventricular hypertrophy can occur in various morphologies resulting in a wide array of clinical manifestations and hemodynamic abnormalities.

The most common form of valvular left ventricular outflow tract obstruction is aortic stenosis. Aortic stenosis is a common disease that usually affects older patients. Aortic stenosis is a narrowing of the aortic valve opening. Aortic stenosis restricts the blood flow from the left ventricle to the aorta and may also affect the pressure in the left atrium. Although some people have aortic stenosis because of a congenital heart defect called a bicuspid aortic valve, this condition more commonly develops during aging as calcium or scarring damages the valve and restricts the amount of blood flowing through the left ventricular outflow tract.

The most common form of supravalvular left ventricular outflow tract obstruction is a narrowing (stenosis) of the large blood vessel that carries blood from the heart to the rest of the body (the aorta). The condition is described as supravalvular because the section of the aorta that is narrowed is located just above the valve that connects the aorta with the heart (the aortic valve).

In addition to aortic stenosis, other heart valves can become compromised. Degenerative valvular heart disease includes aortic valve stenosis, mitral regurgitation, aortic regurgitation, and mitral stenosis. The diagnosis and evaluation of valvular disease is challenging due to various pitfalls. These include discrepancies between the severity of valve dysfunction and patient symptoms. In addition, a number of confounding factors and technical issues affect the assessment, including the presence of concurrent conditions such as uncontrolled hypertension, rapid atrial fibrillation, left ventricular dysfunction, and other valvular dysfunctions.

Current Screening and Diagnostic Approaches. The diagnosis of structural and valvular abnormalities in the left ventricular outflow tract is complicated and typically involves a direct assessment of the heart or the flow characteristics during systole. Given the prevalence of aortic stenosis, this summary of standard tests will be aortic stenosis centric, but the tests listed can be used for other abnormalities. (Zakkar, M., Alan J. Bryan, and G. D. Angelini. “Aortic stenosis: diagnosis and management.” Bmj 355 (2016).)

One common test is auscultation, in which a healthcare provider listens to the heart with a stethoscope to determine if any abnormal sounds may indicate aortic stenosis. However, there are several limitations to the use of auscultation for this purpose. One limitation is that the accuracy of auscultation for diagnosing aortic stenosis can vary depending on the individual's body habitus and the presence of other conditions that may affect the sounds heard through the stethoscope. For example, obesity or the presence of lung disease can make it more difficult to interpret the sounds heard during auscultation accurately.

Another limitation is that the diagnostic accuracy of auscultation for aortic stenosis may be affected by the skill and experience of the healthcare provider performing the test. Auscultation requires the ability to recognize and differentiate between normal and abnormal heart sounds, and this can be challenging for healthcare providers who are less experienced in this area.

Overall, while auscultation can be a useful tool for diagnosing aortic stenosis, it should be combined with other tests, such as echocardiography or cardiac catheterization to ensure an accurate diagnosis.

Echocardiography, or ultrasound, is the most common test used to diagnose aortic stenosis. This test uses sound waves to create a visual image of the heart and can provide detailed information about the size and function of the aortic valve. However, the accuracy of echocardiography may be limited in cases where the patient has poor acoustic windows or when there are calcifications or other abnormalities in the aortic valve that can affect the accuracy of the image.

Magnetic resonance imaging (MRI) is another test that can be used to diagnose aortic stenosis. This test uses a powerful magnetic field and radio waves to create detailed images of the heart and surrounding structures. While MRI is generally very accurate and provides detailed images, it is not always feasible for patients with certain medical implants or conditions that may be affected by the magnetic field.

Cardiac catheterization, also known as angiography, is a more invasive test that involves inserting a small tube (catheter) into a blood vessel and injecting a contrast agent to visualize the blood vessels and heart. This test can provide detailed information about the anatomy of the aortic valve and the blood flow through it. However, it does involve some risks and is generally reserved for cases where other tests are inconclusive or when treatment is being planned.

Computed tomography (CT) is another test that can be used to diagnose aortic stenosis. This test uses x-rays to create detailed images of the heart and surrounding structures. While CT is generally very accurate and provides detailed images, it does expose the patient to radiation and may not be suitable for patients who are pregnant or have certain medical conditions. A comparison of the diagnostic modalities is shown in FIG. 3 and includes catheterization, CT, and MRI.

Due to the difficulty of detecting left ventricular outflow tract obstructions via clinical exam and the cost associated with ultrasound determination, there is a need for a simple screening test that can be administered in the patient's home or the outpatient clinic.

### Disclosure of the Invention

Embodiments of the present invention provide an apparatus for determining the presence and quantification of a structural or valvular heart abnormality in the left ventricular outflow tract of a patient, comprising: a peripherally attached noninvasive speckle sensor system comprising one or more optical sensors configured to measure an optical pulse plethysmogram sensitive to both the systolic pulse wave and reflected pulse waves; a sensor control system configured to operate the noninvasive speckle sensor system during a measurement period comprising at least one cardiac cycle to produce a measurement signal during the systolic and diastolic phases of the at least one cardiac cycle; a left ventricular outflow tract assessment system comprising a programmed data processor and memory, wherein the programmed data processor is programmed to implement a mapping function between the measurement signal and the presence or absence of a left ventricular outflow tract anomaly; a left ventricular outflow tract reporting system configured to report the presence or absence of a left ventricular outflow tract abnormality. In addition to furnishing classification information regarding LVOT, the system can offer a quantitative result associated with the abnormality, facilitating precise assessment and ongoing monitoring of the cardiac disease state.

In some embodiments, the programmed data processor is programmed to use morphologic waveform analysis to create a prediction model, and to use the prediction model to determine the presence or absence of a left ventricular tract anomaly. In some embodiments, the programmed data processor is programmed to use a matching model to determine the presence or absence of a left ventricular tract anomaly. In some embodiments, the programmed data processor is programmed to use a set of parameters that define a mapping function between the measurement signal and the left ventricular outflow tract abnormality. In some embodiments, the programmed data processor is programmed to use a set of parameters defining a mapping function between the measured signals and the area of the aortic valve and to determine the quantitative severity of aortic stenosis.

In some embodiments, the sensor control system is configured to operate the sensor system during a measurement period comprising a period during which the patient performs one or more volitional maneuvers, and wherein the left ventricular outflow tract assessment system is configured to determine the presence or absence of a left ventricular outflow tract anomaly based on the measurement signal. In some embodiments, the left ventricular outflow tract assessment system is configured to determine the presence or absence of a left ventricular outflow tract anomaly based on morphologic waveform analysis of the measurement signal.

Embodiments of the present invention provide an apparatus for determining the presence of a left ventricular outflow tract abnormality of a patient, comprising: an optical measurement system comprising (i) one or more optical emitters configured to emit light toward a measurement region of the user and (ii) one or more detectors configured such that light reaches the detectors from the one or more emitters after the light from the emitters has interacted with the measurement region; a sensor control system configured to operate the sensor system at a first set of operational parameters to detect changes in blood flow or blood volume to produce a first measurement signal that is sensitive to the systolic pulse wave and reflected pulse waves during at least one cardiac cycle; a left ventricular outflow tract assessment system comprising a programmed data processor and memory, wherein the programmed data processor is programmed to implement a mapping function between the first measurement signal and the presence or absence of a left ventricular outflow tract abnormality; a left ventricular outflow tract reporting system configured to report the presence or absence of a left ventricular outflow tract abnormality.

In some embodiments, the noninvasive flow sensor system is a speckle plethysmograph. In some embodiments, the noninvasive flow sensor system is a photo plethysmograph. In some embodiments, the noninvasive flow sensor system comprises a speckle and a photo plethysmograph. In some embodiments, the left ventricular outflow tract assessment is configured to use morphologic waveform analysis to create a prediction model, and to use the prediction model to determine the presence or absence of a left ventricular tract anomaly. In some embodiments, the left ventricular outflow tract assessment is configured to use a matching model to determine the presence or absence of a left ventricular tract anomaly.

Embodiments of the present invention provide a method for determining the presence of structural or valvular heart abnormalities in the left ventricular outflow tract of a patient, comprising: providing a noninvasive optical sensor system configured to detect changes in blood volume or blood flow in a measurement region of the user, where the changes are sensitive to the systolic pulse wave and reflected pulse waves; mounting the sensor in optical communication with a peripheral location of the body; acquiring a measurement signal from the noninvasive sensor system during a data acquisition period of at least one cardiac cycle of the patient to obtain an optical pulse plethysmogram; determining the presence of left ventricular outflow tract obstruction by evaluation of the optical pulse plethysmogram using a left ventricular outflow tract assessment system; providing the output of the left ventricular outflow tract assessment system to a user.

Embodiments of the present invention provide a method for estimating the aortic valve area, comprising: providing a noninvasive optical sensor system configured to detect blood flow in a peripheral measurement region of the user; mounting the sensor in optical communication with the skin; acquiring a measurement signal from the noninvasive sensor system during a data acquisition period of at least one cardiac cycle of the patient to obtain an optical pulse plethysmogram; estimating the aortic valve area by evaluation of the optical pulse plethysmogram using a left ventricular outflow tract assessment system; providing the output of the left ventricular outflow tract assessment system to a user.

Example embodiments of the present invention provide a method of determining the presence of preload independence and the presence of cardiac vagal control, comprising: (a) providing a noninvasive sensor configured to detect changes in blood flow in a measurement region of the user; (b) providing a sensor control system configured to operate the noninvasive sensor at operational parameters to acquire a measurement signal; (c) providing a physiological assessment system configured to determine the presence of a preload independence and cardiac vagal control from the measured signal; (d) providing a left ventricular outflow tract assessment system configured to determine the presence of a left ventricular outflow tract abnormality and calculate a quantitative assessment of the abnormality; (e) providing the output of the left ventricular outflow tract assessment system to a user.

Example embodiments of the present invention provide a method for determining a repeatable cardiac state of a user, comprising: (a) acquiring a first measurement signal from a noninvasive sensor configured to detect changes in blood volume or flow in a measurement region of the user, where changes contain systolic time interval information; (b) providing a physiological assessment system configured to analyze the measured systolic time interval information to determine the presence of a repeatable cardiac state; (c) providing a qualification or validation system configured to indicate the presence of a repeatable cardiac state as determined by the physiological assessment system; (d) providing a left ventricular outflow tract assessment system configured to analyze the optical pulse plethysmogram to determine a quantitative assessment of aortic stenosis; (e) providing a quantitative assessment of aortic stenosis to user.

In some embodiments, the left ventricular outflow tract assessment system further considers ancillary information in determining the presence of left ventricular outflow tract obstruction. In some embodiments, the optical measurement system uses pulse enhancement techniques to improve signal quality. Some embodiments further comprise causing the patient to perform one or more volitional patient maneuvers to alter venous return during the data acquisition period. In some embodiments, the one or more volitional patient maneuvers comprises a change in body position Some embodiments further comprise providing a resistance breathing device, and wherein the one or more volitional patient maneuvers comprises the patient using the resistance breathing device. In some embodiments, the peripheral location of the body is a finger. In some embodiments, the peripheral location of the body is the ear or ear canal. In some embodiments, the peripheral location of the body is the retinal of the eye. In some embodiments, the left ventricular outflow analysis system comprises a prediction model that maps the optical pulse plethysmogram to the type of left ventricular outflow tract abnormality. In some embodiments, the prediction model comprises multiple hierarchical layers. In some embodiments, providing an optical measurement system comprises providing a noninvasive speckle sensor system sensitive to flow in peripheral arteries during both systolic and diastolic phases of the cardiac cycle.

### BRIEF DESCRIPTION OF DRAWINGS

FIG. 1 is an illustration of aortic stenosis and the flow impacts.

FIG. 2 is a figure illustrating the silent progression of aortic stenosis.

FIG. 3 is a table of current methods used to access aortic stenosis.

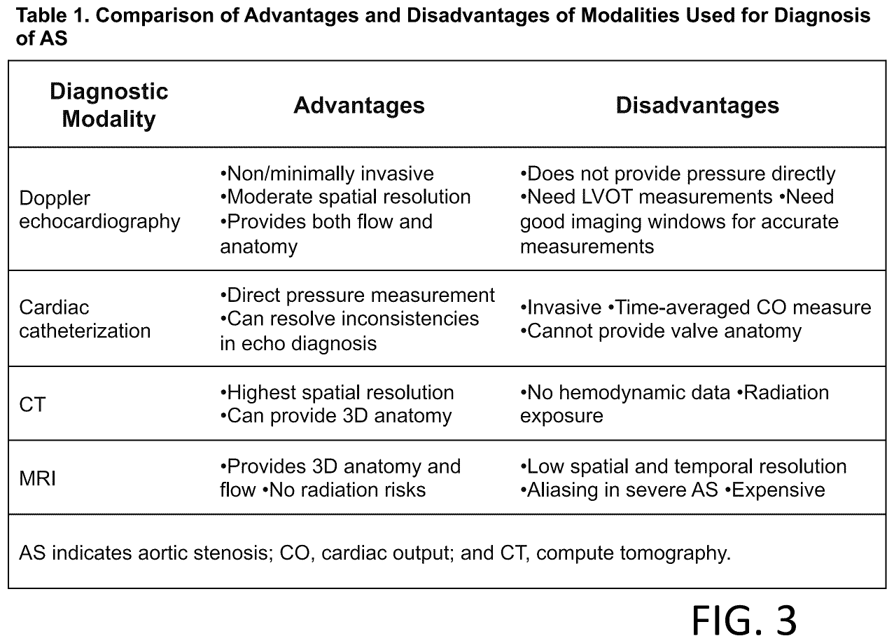

FIG. 4 is a graph showing the differences between moderate and severe aortic stenosis.

FIG. 5 is a table of physical examination findings to differentiate various causes of LVOT obstruction.

FIG. 6 is a pictorial representation of the aortic valves used in the in silico simulation.

FIG. 7 is an in silico simulation of aortic stenosis at peripheral sites illustrating pressure and PPG waves.

FIG. 8 is an in silico simulation of aortic stenosis at peripheral sites illustrating flow and PPG waves.

FIG. 9 is an illustrative example of the impact of vascular aging on pulse waveforms.

FIG. 10 is an illustration showing the impact of vasoconstriction and vasodilation on reflected waves.

FIG. 11 is an illustration showing the size and shape impacts of changes in vascular tone.

FIG. 12 is an illustration of a finger based speckle measurement system.

FIG. 13 is a graph showing representative SPG and PPG waveforms.

FIG. 14 is an example of an eye imaging system using a mobile phone.

FIG. 15 is an illustration of sampling in the ear canal with a pulse plethysmography system.

FIG. 16 is an illustration of the impact of controlled breathing on cardiac function.

FIG. 17 is a flow chart showing how volitional maneuvers can be used to improve diagnostic information.

FIG. 18 is an illustration showing the impact of positional changes on blood distribution.

FIG. 19 illustrates the relationships between pressure and volume during the cardiac cycle.

FIG. 20 shows the relationships between EKG, PCG, and PPG and their associated time intervals.

FIG. 21 shows the impact of hydrostatic pressure differences on pulse size.

FIG. 22 shows the impact of hydrostatic pressure differences on pulse size.

FIG. 23 shows the Frank-Starling curve and the location of preload independence.

FIG. 24 illustrates other passive leg-raising maneuvers.

FIG. 25 illustrates a coordinate system for the assessment of cardiac vagal control.

FIG. 26 illustrates a method for determining LVOT abnormality.

FIG. 27 is an illustration showing outflow tract disease progression in two patients.

FIG. 28 is a diagram showing the measurement configuration elements.

FIG. 29 is a flow chart of an example measurement process.

FIG. 30 is a system configuration diagram.

FIG. 31 is a pictorial representation of the finger SPG measurement configuration.

FIG. 32 is the detailed measurement configuration associated with the finger SPG measurement.

FIG. 33 is a pictorial representation of the ear PPG measurement configuration.

FIG. 34 Is the detailed measurement configuration associated with the ear PPG measurement configuration.

FIG. 35 is a diagram showing a potential breathing protocol.

FIG. 36 is a pictorial representation of the eye laser contrast speckle imaging configuration.

FIG. 37 is the detailed measurement configuration associated with the eye laser contrast speckle imaging configuration.

FIG. 38 is a pictorial representation of the dual eye measurement configuration.

FIG. 39 is the detailed measurement configuration associated with the dual eye measurement configuration.

FIG. 40 is an optical system layout enabling dual sampling of the retina.

FIG. 41 is a pictorial representation of the chest PPG measurement configuration.

FIG. 42 is the detailed measurement configuration associated with the chest PPG measurement configuration.

FIG. 43 is a pictorial representation of the face iSPG measurement configuration.

FIG. 44 is the detailed measurement configuration associated with the face iSPG measurement configuration.

FIG. 45 is a detailed measurement configuration illustrating the ability to create different measurement system based on measurement needs.

FIG. 46 is an embodiment of both spg and ppg sampling at the wrist.

FIG. 47 is a schematic of a closed loop 1D/0D blood flow model of the entire cardiovascular system.

FIG. 48 is a table summarizing the hemodynamic characteristics of all healthy subjects.

FIG. 49 illustrates the substantial variability in pulse waveforms across all healthy subjects.

FIG. 50 compares a selection of simulated pulse waves to in vivo pulse waves.

FIG. 51 compares the hemodynamic characteristics of the simulated pulse waves to in vivo pulse waves.

FIG. 52 is a table summarizing the hemodynamic characteristics of all 75-year-old subjects.

FIG. 53 is simulated pressure and flow pulse waves for the 75-year-old baseline subject.

FIG. 54 depicts simulated Reynolds number and peak aortic flow across the left aortic valve for all subjects.

FIG. 55 plots SPG and PPG pulse waves at the radial artery with variance in age between 50 and 75.

FIG. 56 plots SPG and PPG pulse waves at the radial artery with variance of cardiovascular properties.

FIG. 57 plots SPG and PPG pulse waves at the radial artery with variance orifice area and constant age.

FIG. 58 plots SPG and PPG pulse waves at the carotid artery with variance in age between 50 and 75.

FIG. 59 plots SPG and PPG pulse waves at the carotid artery with variance of cardiovascular properties.

FIG. 60 plots SPG and PPG pulse waves at the carotid artery with variance orifice area and constant age.

FIG. 61 is a pictorial representation of a neural network.

FIG. 62 is the analysis results for flow predictions utilizing simulated waveforms at the radial artery.

FIG. 63 is plot of the sensitivity against specificity for flow predictions at the radial artery.

FIG. 64 is the analysis results for volume predictions utilizing simulated waveforms at the carotid artery.

FIG. 65 is plot of the sensitivity against specificity for volume predictions at the carotid artery.

FIG. 66 is a graph of Reynolds number across aortic valve for simulated subjects.

### BRIEF DESCRIPTION OF THE INVENTION

Embodiments of the invention provide apparatuses and methods for determining and quantifying structural and valvular abnormalities of the left ventricular outflow tract from noninvasive, peripherally obtained optical pulse plethysmogram. The invention does not require direct imaging of the heart or invasively obtained pressure and flow measurements but rather uses peripherally obtained noninvasive measurements of flow, volume, or a combination thereof. The invention addresses historical limitations of auscultation for screening and diagnosis of structural and valvular abnormalities in the left ventricular outflow tract by (1) utilizing improved optical measurement systems, (2) sampling locations that maximize the information content of the pulse waveform, (3) conducting volitional patient maneuvers to improve the diagnostic ability of the system, (4) utilizing pulse enhancement techniques, (5) eliminating necessity of an audible murmur, (6) elimination operator specific determination, and (7) defining a repeatable cardiac state that improves measurement accuracy. Unlike many other diagnostic methods that focus solely on the early systolic phase of the cardiac cycle, the invention can utilize the information for both the systolic and diastolic phases of the cardiac cycle.

### DEFINITIONS

### General Terms

Measuring or measurement process, as used herein, refers to the process of obtaining a signal from a sensor.

A measurement signal or measured signal, as used herein, is the raw data or information obtained from the sensor system during a measurement process. Analysis and assessment systems process measurement signals for desired measurement results.

A parameter, as used herein, is a value that characterizes, summarizes, defines, or describes the properties of an entity. For example, a parameter can be calculated from a measurement signal to describe the properties of the signal. A parameter can also describe the properties of an individual (e.g., age, gender, weight, or the presence of a medical condition).

A plethysmogram, as used herein, is the time series of data enabling a graphical representation of either blood volume or blood flow changes over time. In this specification, a plethysmogram is synonymous with a waveform. A plethysmograph is a device that measures, creates, and records plethysmograms. Plethysmography is the process of using a plethysmograph to measure and record plethysmograms.

Classification, as used herein, is a systematic method of categorizing heart abnormalities based on shared characteristics or properties. This process involves grouping similar abnormalities into distinct categories or classes, facilitating diagnosis, treatment, and research in the field of cardiology.

Risk score, as used herein, denotes a probability value derived from predictive models that provides an indicator score indicating a likelihood that the patient is at risk of or suffering from the cardiac disease using a predictive model. It is utilized primarily in healthcare settings to guide clinical decisions, enabling healthcare providers to identify individuals at higher risk of disease, and to tailor prevention and treatment strategies accordingly. The predictive model is configured to receive the plurality of features as inputs and is trained on a corpus of training plethysmography signals according to a multiple instance learning via embedded instance selection (MILES) approach with yet-another-radial-distance-based-similarity (YARDS) measure.

Quantitative result, as used herein, denotes a quantitative result generated by computation on measurement or input data. These quantitative results offer precise and quantifiable estimation of a physical attribute. For example, the estimation of aortic valve area. In the realm of cardiac assessment, quantitative analysis plays a fundamental role in defining the physical attributes of the disease condition. A quantitative result is distinguished from a risk score as it quantifies the presence of disease rather than the likelihood of acquiring or having the disease.

Qualification, qualifying, and validation, as used herein, refer to the processes of ensuring that equipment, procedures, individuals, physiological states, signal-2-noise ratios, etc meet predetermined criteria (qualification), the act of demonstrating suitability for a specific purpose (qualifying), and the comprehensive testing to confirm that a system, process, or result produces an output meeting pre-defined acceptance criteria (validation).

### Sensors and Signals

Noninvasive sensors, as used herein, refers to a class of sensors that can be used outside the body and are sensitive to blood flow and blood volume, cardiac function, and physiological signals.

Electrocardiogram, as used herein, is a test that records the electrical activity of the heart. The measured signals can be used in both physiological assessments and the determination of cardiac fitness.

Phonocardiogram, as used herein, is a recording of the sounds made by the heart and are related to the mechanical activities of the heart. The measured signals can be used in both physiological assessments and the determination of cardiac fitness.

Seismocardiogram, as used herein, is a technique for recording and analyzing cardiac vibratory activity as a measure of cardiac contractile functions. The measured signals can be used in both physiological assessments and the determination of cardiac fitness.

Ballistocardiography, as used herein, is a technique for producing a graphical representation of the reaction of the body to cardiac ejection forces or the reaction of the body to the blood mass ejected by the heart with each contraction associated with arterial circulation. The measured signals can be used in both physiological assessments and the determination of cardiac fitness.

Vibrational and acoustic measures, as used herein, refers to those measurement technologies that are sensitive to the vibration generated by the heart or blood flow and include phonocardiogram, seismocardiogram, ballistocardiography, or any other method that is sensitive to the vibrations or sound created by the heart.

Echocardiography, as used herein, is the use of ultrasound to investigate the action and functioning of the heart. The measured signals can be used in both physiological assessments and the determination of cardiac fitness.

Speckle plethysmograph (SPG), as used herein, is a noninvasive optical measurement system that measures blood flow in the body. The system uses a laser or other light source to illuminate the skin and tissue, and then analyzing the scattered light patterns, or speckles, which are produced. The system can operate in reflection sampling mode and transmission and transmission sampling mode. The system can be used to measure blood flow in various parts of the body, such as the hand, finger, wrist, foot, or brain, and can provide important information about the function of the circulatory system and the health of tissues and organs. A speckle sensor system creates a plethysmogram representing changes in blood flow over the cardiac cycle.

Photo plethysmograph (PPG), as used herein, is an optical measurement system that measures changes in blood volume using changes in light absorption and can be used to measure blood volume in a transmission sampling mode and reflection sampling mode. The measured signals, a plethysmogram, can be used to calculate both physiological and cardiometric parameters for both physiological assessments and the determination of cardiac fitness. A PPG system creates a photo plethysmogram representing changes in blood volume over the cardiac cycle.

Radar plethysmograph (RPG) is a noninvasive millimeter-wave, radar-based device for the accurate measurement of arterial pulse waveforms. Radar plethysmography can be utilized at any location on the body where a pulse creates a detectable movement of the skin or tissue. A common location is to use the system as a wrist-worn device that positions the radar near the radial artery without touching the skin, allowing for interrogation of the pulse at close range without perturbing the pulse waveform.

Optical sensors, as used herein, refers to any optically based system that can be used to capture signals related to changes in blood volume, flow, or pressure in a measurement region of the individual, which changes are indicative of cardiac function.

Optical pulse plethysmogram, as used herein, is the graphical representation and associated data stream or signal obtained noninvasively via the use of optical systems that are sensitive to changes in blood volume or blood flow in a region of the body resulting from cardiac activity over the entire cardiac cycle.

Speckle plethysmogram, as used herein, is the graphical representation and associated data stream or signal obtained noninvasively via the use of optical systems that are sensitive to changes in blood flow in a region of the body resulting from cardiac activity over the entire cardiac cycle.

Photo plethysmogram, as used herein, is the graphical representation and associated data stream or signal obtained noninvasively via the use of optical systems that are sensitive to changes in blood volume in a region of the body resulting from cardiac activity over the entire cardiac cycle.

Noninvasive optical sensor system, as used herein, is a noninvasive system for acquiring optical pulse plethysmogram data.

Ancillary information, as used herein, defines additional information used in the measurement process to include demographic parameters, health status measures, and other additional information that allows a more accurate and meaningful assessment. Ancillary information can include blood pressure, heart rate, stroke volume, left ventricular ejection time, age, gender, health status, height, weight, medications, mean arterial pressure, pulse wave velocity, body position and arterial diameter.

Ancillary signals, as used herein, pertain to different measurement methods and signals that provide additional information regarding cardiac or respiratory function. Examples include but are not limited to electrocardiogram, phonocardiogram, seismocardiogram, ballistocardiography, anemometry, and spirometry.

### Volitional Activities

Volitional patient maneuvers, as used herein, are volitional actions performed by the patient that modify cardiac performance in a deterministic fashion by changing filling pressure, afterload pressure, or heart rate.

Resistance breathing, as used herein, refers to any breathing method that increases, decreases, or changes intrathoracic pressure over normal breathing and alters venous return. A resistance breathing test can include inhalation resistance breathing or exhalation resistance breathing, independently or in combination. Resistance breathing is a method that can be used to change venous return to the heart and influences end-diastolic volume.

Paced breathing, as used herein, is a general term that applies to any method that defines a breathing rate and can include depth of breathing.

Controlled breathing, as used herein, is the process of combining elements of paced breathing with resistance breathing.

Continuous Positive Airway Pressure (CPAP), as used herein, describes a system that maintains a constant positive pressure throughout the breathing cycle.

Hydrostatic positional change, as used herein, is a general term that applies to any process that changes the hydrostatic pressure in a vessel due to positional changes.

### Sampling Terms and Locations

Vascular tone, as used herein, are changes in the size of the arteries that impact both the shape and the size of the arterial pulse waveform. Locations with reduced vascular tone changes include locations with highly consistent perfusion and reduced sensitivity to autonomic changes relative to peripheral sites such as the wrist and finger. Such locations are typically those supplied by the arterials supporting the brain. Specific examples include the ear canal, tympanic membrane, retinal vessels, inter-month vasculature, and the nasal septum.

Peripherally located arteries, as used herein, refer to those arteries located outside the thorax cavity. These arteries are closer to the skin surface, making them more accessible for examination and evaluation compared to central arteries. Peripheral arteries are in the neck, arms, legs, and on the skin surface of the thorax. In contrast, central arteries are located within the thorax, deeper within the body, and are surrounded by other vital organs. Additionally, the turbulence present in central arteries near the heart with aortic stenosis is not present in the peripherally located arteries. The Reynolds numbers in these peripheral arteries do not support turbulent flow.

Tri-layered vessels, as used herein, refers to blood vessels comprised of three layers: the tunica intima, the tunica media, and the tunica adventitia. Tri-layered vessels include arteries, arterioles, venules, and veins but do not include capillaries, which are comprised of a single layer of endothelial cells.

Transmission dominant sampling, as used herein, refers to optical sampling of the tissue where the majority of photons penetrate and travel through the tissue, interacting with (i.e., reflected by, scattered by, or absorbed by) tri-layered vessels.

Transmural pressure adjustment, as used herein, refers to the ability to adjust the transmural pressure at the sight of sampling based on anatomical and physiological characteristics. For example, the physical size of the finger can impact the pressure exerted by a spring, and the patient's blood pressure will impact the ideal transmural pressure for optimal signal quality. Additionally, changes in the hydrostatic pressure will impact the transmural pressure, such as raising or lowering the arm.

Peripheral sampling location(s), as used herein, refers to the areas of the body that are outside the inter thorax, and include arteries, arterioles, and capillaries at or near the skin surface and typically located in limbs and extremities. Specific sampling location includes the carotid artery in the neck, the capillary bed of the inner ear, the capillary bed of the finger, the radial or ulnar arteries, and the digital arteries.

### Medical Terminology

Left ventricular outflow tract (LVOT), as used herein, is the anatomical structure through which the left ventricular stroke volume passes towards the aorta.

Audible murmur, as used herein, is an audible manifestation of turbulent blood flow during the heart's systolic phase, primarily caused by the acceleration of blood through a narrowed valve or pathologic change in the heart's anatomy. These murmurs arise from the principles of fluid dynamics, particularly through the concept of Reynolds number. This dimensionless number predicts when flow transitions from laminar to turbulent, based on variables such as flow velocity, fluid density, viscosity, and the diameter of the vessel. When blood accelerates through a narrowed valve during systole, exceeding the critical Reynolds number, it transitions to turbulent flow, producing vibrations. These vibrations, generated by the chaotic movement of blood, propagate through the heart and thorax, becoming audible as murmurs when they reach the chest wall. Thus, the characteristic sounds of these murmurs, heard during auscultation, directly result from turbulent blood flow, offering insights into the severity of valve obstruction.

Structural and valvular heart abnormalities in the left ventricular outflow tract, as used herein, refer to abnormalities that create deviations from normal left ventricular flow. Structural obstructions are commonly defined based on the location of the obstruction and include entities located at the aortic valve level (valvular), in the ascending aorta (supravalvular), and in the proximal portion of the left ventricular outflow tract (subvalvular). Valvular abnormalities can include aortic valve stenosis, mitral regurgitation, aortic regurgitation, and mitral stenosis.

Left ventricular outflow tract anomaly, as used herein, is any abnormality that impacts blood flow during ventricular contraction.

The systolic pulse wave, as used herein, is the pulse wave that is generated by the contraction of the left ventricle of the heart. When the left ventricle contracts, it pumps blood out into the aorta, creating a pressure wave that moves from the aortic valve to the ascending aorta and into the periphery.

The reflected pulse waves or reflected waves, as used herein, are waves that are generated when blood flows through the arteries in the human body. As the blood flows through the arteries, it encounters bifurcations, elasticity changes, and changes in the diameter of the arterial lumen, which can cause the pulse wave to be reflected.

Arterial tree transformations, as used herein, are the changes in the shape of the aortic pulse waveform that occur as the pulse travels away from the heart. The transformation of the pulse waveform is due to many parameters, including the length of travel, the pressure in the vessel, and the characteristics of the conduit vessel, including stiffness.

Patient-specific transformations, as used herein, are those transformations influenced by the anatomical and functional characteristics of the individual. Anatomical differences can refer to the vessels' size, length, and shape. Functional differences can include differences in stiffness/elasticity. These functional properties are influenced by age, gender, the presence of hypertension, and other disease conditions.

Early systole refers to the time from the aortic opening until the point of maximum flow. has been obtained.

Late systole refers to the time from the point of maximum flow to the closure of the aortic valve.

A pulse waveform, as used herein, is generic for waveforms resulting from cardiac activity and is not singularly associated with pressure. Pulse waveforms can be associated with pressure, volume, and flow.

Early-Stage Aortic Stenosis, as used herein, refers to a condition characterized by initial pathological changes in the aortic valve area that affect blood flow, but not to the extent of creating turbulent flow resulting in vibrations or sounds at detectable levels at the skin surface, an audible murmur. This stage of aortic stenosis disease progression is marked by a measurable but subtle alteration in the structure or function of the aortic valve, such as slight narrowing or stiffness, which impacts the hemodynamics through the valve. These changes do not create an aubidle murmur. Audible murmurs are typically associated with more advanced aortic stenosis. The detection of aortic stenosis at this early stage is critical for proactive disease management and may offer opportunities for interventions that can delay progression and improve patient prognosis, as well as prevent irreversible damage to the heart.

Peripheral Flow Alterations due to Aortic stenosis, as used herein, are changes in the flow plethysmogram to include but are not limited to: decreased peak systolic velocity, prolonged ejection time, reduced amplitude and blunted peaks, delayed dissipation of flow rate after peak flow, altered dicrotic notch appearance, delay in flow recovery after dicrotic notch, and altered flow dynamics. These listed changes define observable differences in the flow plethysmogram occurring both during systole and diastole.

Repeatable Cardiac State, as used herein, is a resting state defined by the presence of cardiac vagal control and preload independence. An individual in a repeatable cardiac state is resting, unstressed, and has a venous return at or near maximum normal physiological capacity.

Cardiac Vagal Control, as used herein, defines as an autonomic state when the vagus nerve alters interbeat time intervals with high responsivity, precision, and sensitivity. Cardiac vagal control occurs when sympathetic activation is low, and the parasympathetic nervous system exerts greater control over cardiac function (interbeat time interval and contractility) than the sympathetic nervous system.

Preload Independence, as used herein, defines a physiological state where the variations in cardiac filling pressures have minimal effect on stroke volume. Preload independence occurs during conditions of high venous return when the heart is filled at or around natural capacity. The location of the body in a supine position facilitates preload independence by increasing venous return.

Systolic time intervals, as used herein, are one or more calculated or measured parameters that describe the temporal phases of the cardiac cycle. Cardiac-specific systolic time intervals include EMAT (electromechanical activation time), ICT (isovolumic contraction time), PEP (pre-ejection time), heart rate, interbeat interval, and LVET (left ventricular ejection time). Parameters associated with pulse transit times are PTT (pulse transit time) and PAT (pulse arrival time).

Systolic time interval information, as used herein, is information containing temporal descriptions of the phases of the cardiac cycle. Systolic time interval information can be direct measures such as LVET and heart rate or derived measures such as heart rate variability. Measurement signals containing systolic time interval information include PPG, SPG, EKG, phonocardiogram, seismocardiogram, and ballistocardiography. Systolic time interval information can be used by matching models based on algorithms to include artificial intelligence, machine learning, and deep learning methods.

### System Components and Terms

A sensor system, as used herein, refers to software and hardware that measures the physical or electrical characteristics of cardiac function that enables an assessment for left ventricular outflow tract abnormalities. The sensor system enables the sampling and recording of flow, volume, or pressure from a peripheral location. Capabilities of the sensor system can include but are not limited to sampling, conversion, filtering, amplification, signal quality assessment, processing, and recording. For illustrative purposes, a sensor system for optical measurements can be composed of an emitter, detector, power supply, and microcontroller containing one or more CPUs (processor cores) along with memory and programmable input/output peripherals and RAM.

A sensor control system, as used herein, refers to software and hardware that is designed to control one or more elements of the sensor system. In most implementations, the control system regulates the operation of the sensor system based on input or logic. The logic element is often implemented via a microcontroller with associated software representing the logic needed for operation. The sensor control unit may utilize a microprocessor, a programable data processor, or a computer to process and analyze data from the sensors, and to make sensor operational changes or decisions based on the data. The sensor control system can modify the operation of the sensor system to enable different data acquisition modes, change the length of data acquisition, and initiate changes in the sensor used for signal measurement.

A data acquisition system, as used herein, refers to a device and software for collecting and processing data from various sensors or inputs for subsequent analysis.

Data analysis software, as used herein, refers to computer programs or tools designed to interpret, organize, and transform acquired data into meaningful information for additional evaluation.

The data acquisition period, as used herein, is the time duration needed to acquire a measurement signal for subsequent assessment and analysis.

The left ventricular outflow tract assessment system, as used herein, refers to software and hardware that processes measured signals, including ancillary information, if desired, to determine the presence or absence of a left ventricular outflow tract abnormality, may determine the type of abnormality present, and may provide a quantitative assessment of the degree of abnormality.

Morphological waveform analysis, as used herein, is a method to provide insight into the behavior and characteristics of an underlying system by analyzing the time-varying signals produced by that system. By decomposing the signal into its constituent waveform shapes, or morphs, and analyzing the properties and characteristics of these morphs, it is possible to gain a better understanding of the processes and mechanisms that are driving the system. Morphological waveform analysis can be a valuable tool for understanding the behavior of complex systems such as the human circulatory system and for identifying patterns and trends within the signals that are produced during normal and abnormal conditions.

The left ventricular outflow tract reporting system, as used herein, refers to hardware and software that provides information back to the designated person or designated system. The reporting system may use a designated graphical interface or may transmit information via Wi-Fi or Bluetooth to other display or presentation systems.

The designated person, as used herein, can be the patient, medical staff, or provider.

A designated system, as used herein, can include a screen display, printer, secondary data repository, or electronic medical record.

A physiological assessment system, as used herein, refers to software and hardware that processes measured physiological signals, including ancillary information, if desired, to determine the patient's physiological state.

Venous Return Change Evaluation, as used herein, refers to a volitional activity by the patient or an activity done to the patient that creates a change in venous return to the heart. The resulting change in cardiac function is evaluated to determine if preload independence is present.

Mapping function as used herein describes a mathematical relationship between input information, such as pulse plethysmogram data, and output information regarding the presence or absence, type, or severity of a left ventricular outflow tract obstruction. This function is typically defined by a set of parameters learned from historical data during the training process of predictive algorithms. These parameters vary depending on the type of predictive algorithm employed, such as linear regression models, decision trees, neural networks, support vector machines (SVM), random forest, k-nearest neighbors (k-NN), naive bayes, and gradient boosting machines (GBM), and many others. The learning process involves adjusting these parameters to minimize errors between predictions and true values. Once learned, the mapping function can be applied to new input data to make predictions about future events or outcomes. The output of the mapping function can take various forms, including binary outcomes, multi-class classifications, or continuous variables, depending on the nature of the prediction task. Overall, the mapping function facilitates the translation of input data into meaningful predictions, aiding in clinical assessments of left ventricular outflow tract obstruction.

### Detailed Description of the Invention

The invention relates to a simple and noninvasive screening and diagnostic test for the presence of structural and valvular heart abnormalities in the left ventricular outflow tract. Left ventricular outflow tract obstruction is a condition in which there is a blockage or narrowing of the outflow tract of the left ventricle, which is the main pumping chamber of the heart. This narrowing can cause a reduction in the amount of blood that is pumped out of the heart, leading to symptoms such as shortness of breath, chest pain, fatigue, and can lead to heart failure and death.

The invention provides an apparatus and method for the classification of the left ventricular outflow tract abnormality as well as a quantitative assessment of the degree of abnormality. Classification based on the location of the obstruction is provided, subvalvular, valvular and supravalvular. Additionally, the invention can provide a quantitative measure of the abnormality as a measure of the physical restriction, a percent change from normal, or a body adjusted index.

The invention provides apparatuses and methods for determining the presence of early-stage aortic stenosis from a noninvasive, peripherally obtained speckle pulse plethysmogram. The invention is not dependent on the two critical conditions necessary for operation of auscultation-based screening systems for aortic stenosis: the generation of turbulent blood flow by the stenotic condition and the transmission of resulting vibrations or sounds through the thorax at detectable levels. The invention employs speckle plethysmography, a sensitive flow-based measurement technique, enabling the detection of hemodynamic alterations preceding the manifestation of audible turbulence associated with more advanced stenotic conditions. The apparatus quantitatively evaluates blood flow dynamics, facilitating the early identification and assessment of aortic stenosis without necessitating the presence of turbulent flow resulting in an audible murmur. Consequently, this innovative approach transcends the limitations of traditional auscultation, offering an objective, noninvasive screening and diagnostic modality that is not contingent upon the presence of an audible murmur resulting from turbulent blood flow and the subsequent propagation of those vibration through the chest wall.

Historical descriptions and assessments. The standard description and diagnostic tests associated with structural and valvular abnormalities of the left ventricular outflow tract focus on flow or pressure aberrations from a typical or normal waveform during systole. Pressure pulse waveform abnormalities can be detected via invasive catheterization, while flow aberration can be detected by echocardiography.

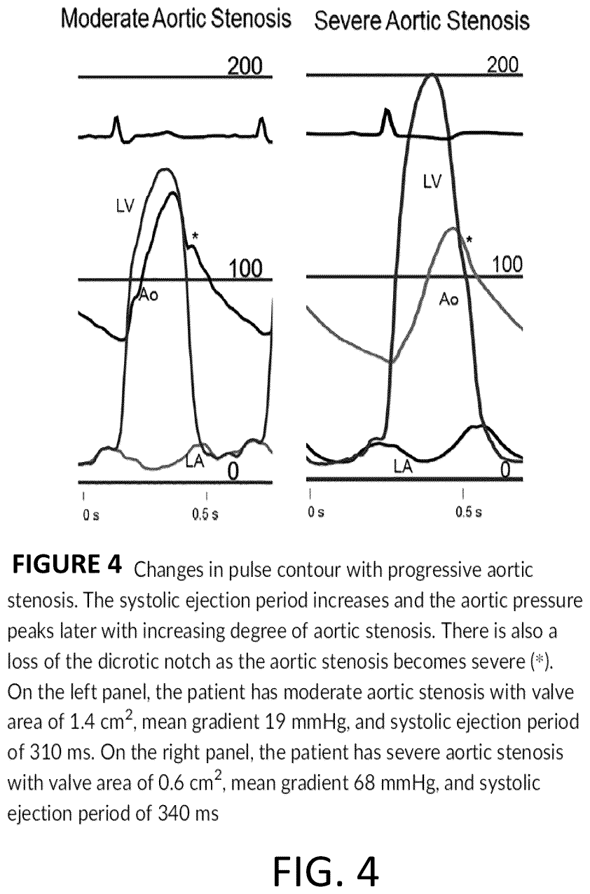

The standard description of aortic stenosis focuses on the systolic phase of the cardiac cycle. A typical description of aortic stenosis describes the pulse waveform in the aorta. Aortic stenosis is characterized by a diminished and delayed aortic pulse. FIG. 4 shows the distinct differences between moderate and severe aortic stenosis. With further stenosis, the ejection period increases, the pressure peaks later, and the dicrotic notch becomes less apparent. It is important to note that the murmur often associated with aortic stenosis is a systolic ejection murmur.

### Auscultation Nuances and Limitations

The screening for aortic stenosis (AS) typically begins with auscultation during a physical exam when a healthcare provider listens to the heart sounds using a stethoscope. The auscultation can reveal the presence of a systolic ejection murmur, a hallmark sign of AS. A systolic ejection murmur is a specific type of heart murmur that occurs during the systole phase of the cardiac cycle, when the heart muscle contracts and pumps blood out of the left ventricle through the aortic valve into the aorta.

The creation of this murmur is attributed to the vibrations caused by turbulence as blood flows across the narrowed aortic valve. This turbulence is not a random occurrence but follows fluid dynamics principles, notably the Reynolds number concept, which predicts the transition from laminar (smooth) to turbulent flow in fluids. In the context of AS, the narrowing of the aortic valve reduces the effective opening through which blood can flow, increasing the velocity of the blood flow to a point where it becomes turbulent, thereby generating the characteristic murmur.

The creation of turbulence is a necessary element, but the resulting vibration must travel though the thorax with enough intensity to create an audible murmur. The human thorax acts as a variable sound conduction system, influencing the transmission and audibility of heart sounds, including murmurs. Factors such as the thickness of the chest wall, the presence of lung tissue between the heart and the chest wall, and even the position of the heart within the thorax can affect how well a murmur is conducted and heard.

Consequently, the screening for aortic stenosis through auscultation is highly variable and depends on several factors. The clinician's experience plays a significant role in accurately identifying and interpreting heart murmurs. Additionally, the physical attributes of the patient, such as body habitus and the presence of other cardiac or pulmonary conditions, can significantly influence the detection of a murmur. The degree of stenosis is also crucial; more severe narrowing is likely to produce a more audible murmur, although this direct linkage is not always observed. Finally, the environment in which the examination occurs can impact the ability to detect murmurs, with noise and distractions potentially obscuring subtle heart sounds.

The performance of auscultation in asymptomatic patients has been well documented and is remarkably poor. The study conducted by Gardezi et al. titled “Cardiac auscultation poorly predicts the presence of valvular heart disease in asymptomatic primary care patients” explored the effectiveness of cardiac auscultation in detecting valvular heart disease (VHD) among asymptomatic patients in primary care settings. (Gardezi, Syed K M, et al. “Cardiac auscultation poorly predicts the presence of valvular heart disease in asymptomatic primary care patients.” Heart 104.22 (2018): 1832-1835.) The sensitivity of auscultation was low for the diagnosis of mild VHD (32%) but slightly higher for significant VHD (44%), with specificities of 67% and 69%, respectively.

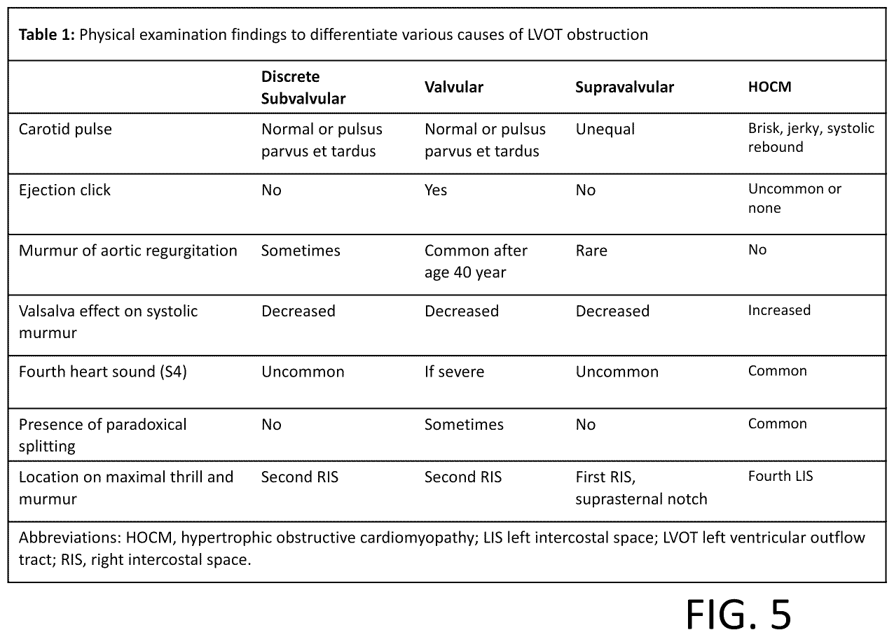

The complexity of using auscultation and physical exam for the classification of left ventricular outflow tract obstruction represents a nuanced and complex diagnostic pathway. FIG. 5 is an example of the various elements of an example pathway for left ventricular outflow trace obstruction and includes hypertrophic obstructive cardiomyopathy. (Devabhaktuni, Subodh R., et al. “Subvalvular aortic stenosis: a review of current literature.” Clinical cardiology 41.1 (2018): 131-136.) As evidenced by the diagnostic table, the information provides a classification but does not quantify the degree of the abnormality.

While auscultation remains a current first step in detecting AS, its nuances and limitations underscore the necessity for improved screening methods. The development of an ideal screening device for aortic stenosis is crucial, one that is not dependent on the operator's skill level and reduces dependencies on patient variances such as thoracic anatomy. This device should be capable of detecting the presence of aortic stenosis with only minimal hemodynamic impact, ensuring early identification of the condition before significant complications arise. By providing a consistent and objective measure of aortic stenosis, such a tool would represent a significant advancement in the screening process, making it more accessible and reliable across diverse clinical environments.

With a desire for more peripheral sampling locations, the use of invasive peripheral pressure measurements might appear as a valid approach to diagnosing aortic stenosis. However, a review of peer-reviewed literature identified only one paper from 1964. Mason et al. reported results from brachial arteries in their report “Diagnostic Value of the First and Second Derivatives of the Arterial Pressure Pulse in Aortic Valve Disease and in Hypertrophic Subaortic Stenosis”. These results were based on absolute pressure measurements obtained from an invasive pressure monitor. The authors conclude that derivative “of the brachial artery pressure pulse afford a simple and reliable assessment of the nature and location of left ventricular outflow obstruction and are helpful in the differentiation of valvular aortic stenosis, combined stenosis and regurgitation, and pure aortic regurgitation.” (Mason, D. T., Braunwald, E., Ross, J., & Morrow, A. G. (1964). Diagnostic Value of the First and Second Derivatives of the Arterial Pressure Pulse in Aortic Valve Disease and in Hypertrophic Subaortic Stenosis. Circulation, 30(1), 90-100.) Again, the focus of the measurement is on the rate of change of the pressure profile during systole. No products or subsequent research papers were identified after the publication in 1964.

### Quantitative Measures of Left Ventricular Aortic Stenosis

Subaortic, aortic, and supra-aortic stenosis are conditions that affect the flow of blood through the heart and the great vessels. They are quantitatively assessed using various measurements to evaluate the severity of the stenosis and guide treatment decisions.

For aortic stenosis, the key measurements include the aortic valve area (AVA), typically measured in square centimeters (cm²), which is directly related to the severity of stenosis. The normal AVA ranges from 5.0 to 3.0 cm², with values less than 1.0 cm² indicating severe stenosis. Aortic Valve Area Index (AVAI) is a measure used to assess the severity of aortic stenosis, adjusted for a patient's body size. It is calculated by dividing the aortic valve area (AVA) by the body surface area (BSA) to normalize the valve size relative to the patient's body size, providing a more accurate assessment of stenosis severity. Additional measures include the peak jet velocity across the valve, measured in meters per second (m/s) using Doppler echocardiography, which estimates the severity of obstruction, with higher velocities indicating more severe stenosis. The mean pressure gradient (MPG), measured in millimeters of mercury (mmHg), is another critical parameter, with higher gradients indicating more significant obstruction. The dimensionless index, which compares the flow rates in the left ventricular outflow tract (LVOT) to that across the aortic valve, is also used, with values less than 0.25 indicating severe stenosis.

For subaortic stenosis, measurements focus on the anatomical and hemodynamic assessment of the left ventricular outflow tract (LVOT). The diameter of the left ventricular outflow tract (LVOT) just below the aortic valve is a key measurement in assessing subaortic stenosis. This measurement is typically obtained using echocardiography, with the LVOT diameter measured in the parasternal long-axis view. The LVOT gradient, measured in mmHg, quantifies the pressure difference between the left ventricle and the aorta. The thickness of the subaortic membrane or the size of the subaortic obstruction, if present, is measured in millimeters (mm) using imaging techniques such as echocardiography or magnetic resonance imaging (MRI).

Supra-aortic stenosis involves the narrowing above the aortic valve, and its quantification may include measurements of the narrowest diameter of the affected vessel, in mm, and the pressure gradient across the stenotic region, measured in mmHg. The flow velocity through the stenotic area, measured in m/s, is also a parameter, with higher velocities indicating more severe obstruction.

### Noninvasive Assessment at Peripheral Locations

In contrast to historical approaches, the invention focuses on the changes occurring over the entire cardiac cycle with a focus on late systole and diastole. Flow variances at the aortic valve are amplified as the systolic pulse wave moves to the periphery due to a combination of the ventricular-aortic interaction, pulse augmentation, and reflected pulse waves. Reflections of the pulse wave play a role in pulse amplification/augmentation in the arterial system. When the pulse wave travels through the arterial system, it encounters a series of branching vessels, changes in vessel diameter, and arterial stiffness. These changes cause reflected pulse waves, leading to the formation of multiple wave fronts.

The reflected pulse waves interact with the forward-moving pulse wave (systolic pulse wave) and amplify or attenuate the pressure and alter the blood flow in the arterial system. This interaction can be complex and depends on many factors, including the magnitude and timing of the reflected pulse waves, the stiffness of the arterial walls, and the flow of blood through the vessels.

Reflections of the pulse wave tend to be more pronounced in arteries with a high degree of compliance (i.e., those that are more elastic and expand more easily). Recent work by Vennin, demonstrated the importance of the aortic root as a major reflection site contributing to the forward wave. (Vennin, Samuel, et al. “Novel pressure wave separation analysis for cardiovascular function assessment highlights the major role of aortic root.” IEEE Transactions on Biomedical Engineering 69.5 (2021): 1707-1716.)

In aortic stenosis, the changes in flow patterns captured by speckle plethysmography are marked by distinct alterations in the waveform, including the presentation and characteristics of the dicrotic notch. Speckle plethysmography, which employs speckle patterns generated by the interaction of light with moving blood to visualize flow, provides a detailed view of these dynamics. The key variances in the flow patterns and the abnormalities in the presence of severe aortic stenosis were examined using in silico simulations.

Evaluation via In Silico Simulations. The in silico simulation of pulse waves has been validated via more than 20 publications and has been used to study the influence of aging, arterial stiffness, and blood pressure. In silico refers to the use of computer simulations or models to predict the behavior of biological systems. The term is derived from the Latin word “in vitro,” which means “in glass,” and refers to experiments that are performed outside of a living organism, such as in a test tube or petri dish. In the context of pulse simulations, in silico experiments are performed entirely on a computer. In silico simulations of pulse waves have been recently used to examine the influence of abdominal aortic aneurysms. (Wang, Tianqi, et al. “Machine learning-based pulse wave analysis for early detection of abdominal aortic aneurysms using in silico pulse waves.” Symmetry 13.5 (2021): 804.)

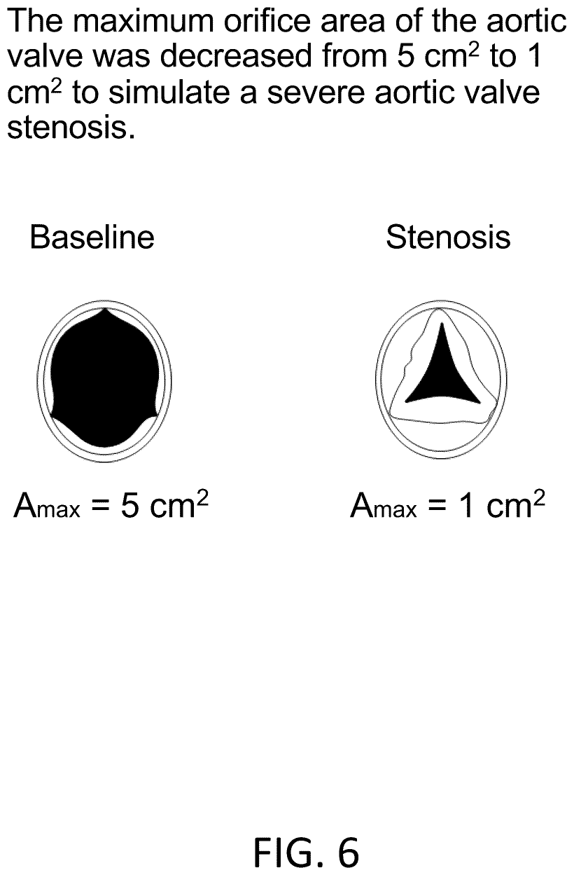

To further explore the ability to make noninvasive, peripherally based determinations of abnormalities in the left ventricular outflow tract, in silico simulations were created for a normal valve and a valve with severe stenosis. The focus of the simulation was to evaluate or confirm that the changes in aortic flow due to stenosis are amplified as the pulse travels to the periphery and that the resulting differences can be detected. Additionally, the in silico simulation allows for the direct comparison of pressure, flow, and PPG signal at proposed sampling sites. The simulation uses a normal valve as the baseline for comparison with a valvular area of 5 cm². The stenosis valve simulated has a valvular area of 1 cm², which is often associated with the development of clinical symptoms. FIG. 6 is a pictorial representation of the aortic valves simulated.

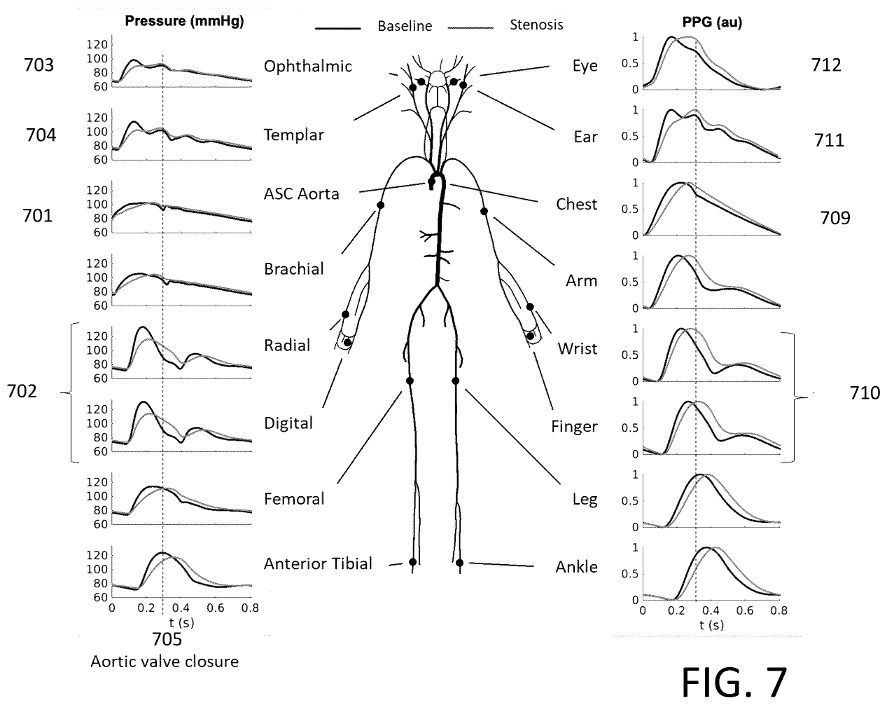

Pressure and PPG Waveforms. FIG. 7 illustrates the results of the in silico analysis for different peripheral sampling sites and shows the resulting pressure profiles and PPG waveforms. Examination of the pressure profile in the ascending aorta, 701, shows agreement with the invasive measurements reproduced in FIG. 4. Specifically, the ejection period increases, the pressure peaks later, the rise in rate of rise in aortic pressure is slower, and the dicrotic notch becomes less apparent.

Examination of the pressure profiles in the radial and digital arteries, 702, shows a more pronounced difference between the normal versus stenotic valve. The pressure rise during early systole is different, as are the pressure waves during late systole and diastole after the closure of the aortic valve, 705. Pressure profiles from the temporal artery, 704, and the ophthalmic artery, 703, show ripples resulting from reflections.

As pressure profiles cannot be easily measured noninvasively, a PPG waveform was simulated for the area near the artery. As PPG is a volume-based measurement based on changes in absorbance, the profiles are similar but have distinct differences. The stenosis PPG profile from the chest, 709, mimics previously reported in-vivo pulse profiles with a limited initial rise and a reduction in the dicrotic notch. PPG waveforms for the wrist and finger, 710, show differences in the pulse waveform, but shapes are similar in form and appear to be time-shifted. The PPG measurements from the eye show differences in the shape during the entire cardiac cycle, 712, and the ear, 711, as shape and inflection differences.

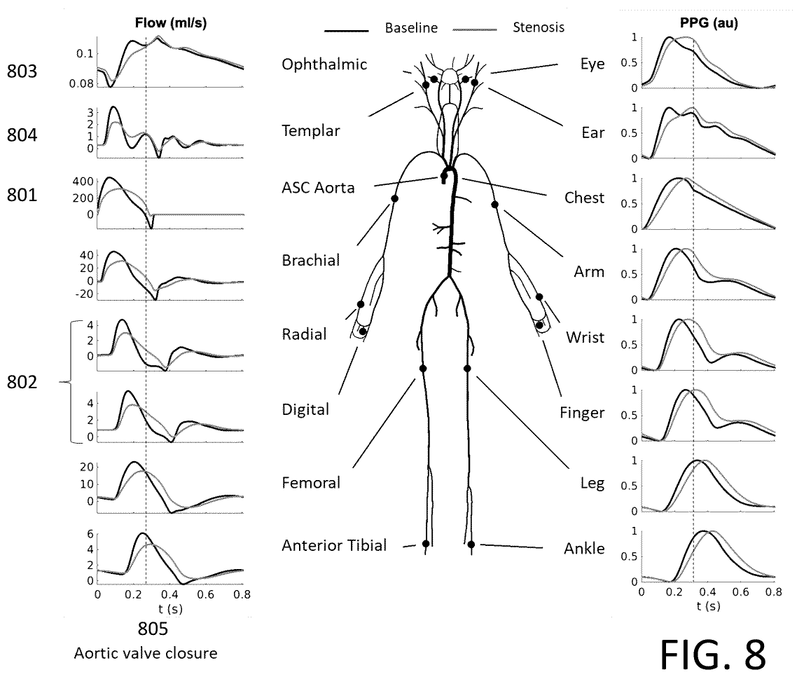

Flow Waveforms. FIG. 8 is similar information but contains information on the flow characteristics on the left side of the page. The inclusion of flow characteristics or flow profiles aids the determination process as it can be measured in the periphery by speckle plethysmograph and generally illustrates more distinct differences when PPG signals. Aortic stenosis impacts flow, so the detection of aortic stenosis by flow bases techniques makes intuitive sense.

Examination of the flow profile in the ascending aorta, 801, shows agreement with the invasive measurements found in published literature. The flow restriction present in stenosis impacts both forward and reverse flow, altering the flow occurring during aortic closure. This flow simulation agrees well with the often visualized loss of the dicrotic notch. Importantly the rate of increase and decrease in flow during systole, 801, is markedly different and will impact aortic root reflections.

Examination of the flow profiles in the radial and digital arteries, 802, shows a visible shape difference between the normal versus stenotic valve. The flow rise during early systole is different with a measurable slope difference. The peak of the location of maximum flow is delayed, and decreasing flow during late systole and diastole is different. In summary, the flow curves of 802, cannot be easily scaled in the x and y dimension to create significant overlap. This fundamental difference in shape can be used to determine the presence of aortic stenosis.

Significant shape and flow rate differences are present in the temporal artery, 804, and the ophthalmic artery, 803. Examination of the flow profiles demonstrates the significant influence of reflections on flow over the cardiac system. Examination of the arterial structure of the head reveals many junctions and a redundant blood supply that creates a complex array of reflections. One of the most complex arterial formations is the circle of Willis. The circle of Willis is a network of blood vessels in the brain that supplies blood to the brain and the brainstem. It is formed by the convergence of several arteries, including the internal carotid arteries and the vertebral arteries. The circle of Willis is an important structure in the body because it provides backup blood supply to the brain in the event that one of the main arteries becomes blocked or damaged. The circle of Willis is a unique structure as most arteries are based on unidirectional flow while the circle of Willis supports bidirectional flow which can result in the collision of forward traveling waves. This complex flow pattern creates the odd flow pattern present in the ophthalmic artery, 803.

### Peripheral Flow Alterations due to Aortic Stenosis

Careful examination of the in-silico simulations at the radial artery demonstrates the following alterations in flow that are indicative of arterial stenosis, FIG. 8, flow plethysmogram 802:

(1) Decreased Peak Systolic Velocity: The restriction caused by the narrowed aortic valve leads to a notable decrease in the peak flow velocity, which is clearly depicted in the flow plethysmogram This reduction reflects the compromised ability of the heart to eject blood efficiently during systole.

(2) Prolonged Ejection Time: The obstruction at the valve prolongs the time it takes for blood to be ejected, resulting in a flow plethysmogram that rises more slowly to its peak. This prolongation indicates the heart's increased workload in overcoming the valve's resistance.

(3) Reduced Amplitude and Blunted Peaks: The flow pattern is more rounded and with a smaller peak value because less blood volume per unit of time can flow through the aortic valve due to a reduced orifice area. This results in rounder and smaller amplitude flow waves throughout the arterial network.

(4) Delayed Dissipation for Flow Rate after Peak Flow: the flow pattern associated with aortic stenosis after peak flow but before the dicrotic notch is very different. Information content associated with aortic stenosis is not isolated to early systole but is present over the entire cardiac cycle, with reflection differences contributing to the shape differences.

(5) Altered Dicrotic Notch Appearance: The dicrotic notch may appear diminished, altered in position and shape due to the changes in aortic pressure, increased left ventricular pressure, and alterations in valve closure. In aortic stenosis, the valve leaflets may become thickened or calcified, limiting their mobility. This reduced mobility can affect the ability of the valve to close efficiently, leading to incomplete closure or restricted movement.

(6) Delay in Flow Recovery after Dicrotic Notch: the recovery of flow is distinctly different after the presence of the dicrotic notch.

(7) Altered Flow Dynamics: The disruption of laminar flow by the stenotic aortic valve can create turbulence in the aorta, observable in the speckle plethysmogram as irregularities or disturbances in the flow pattern in the periphery, particularly beyond the peak systolic point. This flow variance is not an alteration on the flow plethysmogram but a variance in the characteristics of the speckle pattern obtained from the artery.

These stenosis related alterations in the flow plethysmogram are the basis upon which a quantitative evaluation of aortic stenosis can occur. These plethysmogram changes must be differentiated from other normal physiological changes to include age related changes, blood pressure, arterial stiffness, changes in sympathetic tone and filling pressures.

### Pulse Transformations

Between Patient Pulse Wave Transformation. As the aortic pulse waveform travels to the periphery, the waveform is transformed and distorted by the distance of travel in the conduit arteries. The shape transformation occurs due to pulse pressure amplification and reflected waves. Pulse pressure amplification is a phenomenon that occurs when blood flows through a tapered artery. It refers to the increase in pulse pressure, or the difference between systolic and diastolic blood pressure, as the blood flows through the artery. This increase in pulse pressure is caused by the narrowing of the artery, which leads to an increase in arterial resistance and a decrease in arterial compliance. As the blood flows through the narrow section of the artery, it encounters increased resistance and is forced to exert a greater pressure to continue flowing. This results in an increase in systolic blood pressure, while the diastolic blood pressure remains relatively unchanged.

Pulse waveform distortion in arteries is caused by reflections of the pulse wave as it travels through the arterial system. These reflections can occur at points where there are changes in the diameter or elasticity of the arterial walls, or where the arterial tree branches are. When a pulse wave is transmitted through the arterial system, it travels at a certain velocity. If the arterial walls are uniform in diameter and elasticity, the pulse wave will travel smoothly without any significant distortion. However, if there are changes in the diameter or elasticity of the arterial walls, or if the arterial tree branches, the pulse wave will be reflected back towards the heart. These reflections can cause the pulse waveform to become distorted, resulting in changes in the shape and amplitude of the pulse wave.

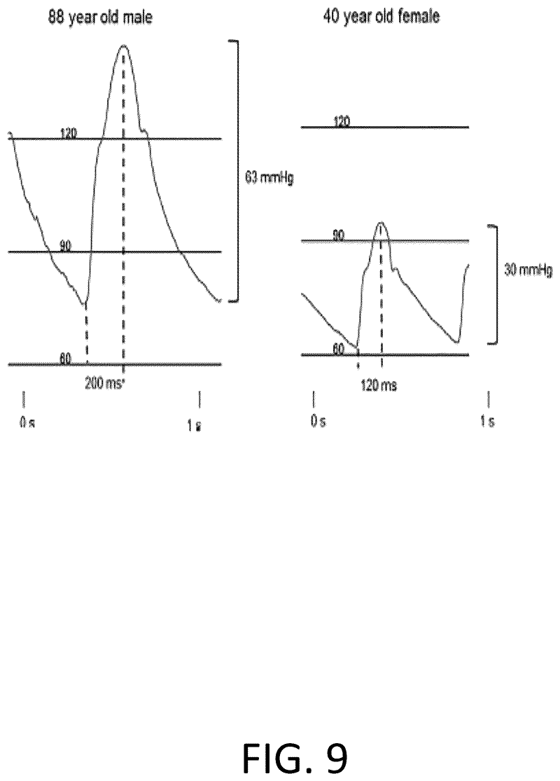

Individual patient differences impact the aortic pulse waveform transformation. Individual differences can be broadly classified as anatomical and functional. Anatomical refers to the size, length, and shape of the vessels. Functional refers to differences in pulse pressure, mean arterial pressure, and the stiffness or elasticity of the vessels. These functional properties are heavily influenced by age, gender, the presence of hypertension, and other disease conditions. Hypertension and increased arterial stiffness result in arterial wave reflections that can significantly alter the aortic pulse waveform. FIG. 9 is an illustrative example of the impact of vascular aging and the stiffening with age that occurs. As can be seen in the figure, the pulse pressure peaks earlier in the younger individual, and the pulse pressure is increased in the older patient. It is important to note that several types of left ventricular outflow tract obstructions create a delayed pressure rise. Thus, an element of the invention is the procurement of signals and information as well as analysis methods that allow the system to determine the difference due to aging or that resulting from a left ventricular outflow tract obstruction. Thus, the invention seeks to mitigate the measurement impact of patient-specific differences Additionally, the left ventricular outflow tract assessment system may use ancillary information to include both anatomical and functional information to compensate for these differences.

Within Patient Pulse Transformations. Within-patient pulse transformations can occur with changes in physiological status. These changes can impact cardiac function resulting in pulse wave changes unassociated with left ventricular outflow tract abnormalities. Physiological parameters include but are not limited to blood pressure, body temperature, breathing rate, interbeat time interval, heart rate, blood oxygen saturation, body position, interbeat time interval variability, cardiorespiratory phase, and sympathetic and parasympathetic tone. The measurement process is facilitated by having the patient in a repeatable cardiac state. A repeatable cardiac state is defined as resting, unstressed, with the presence of cardiac vagal control resulting in respiratory sinus arrhythmia and with venous return at or near maximum capacity.

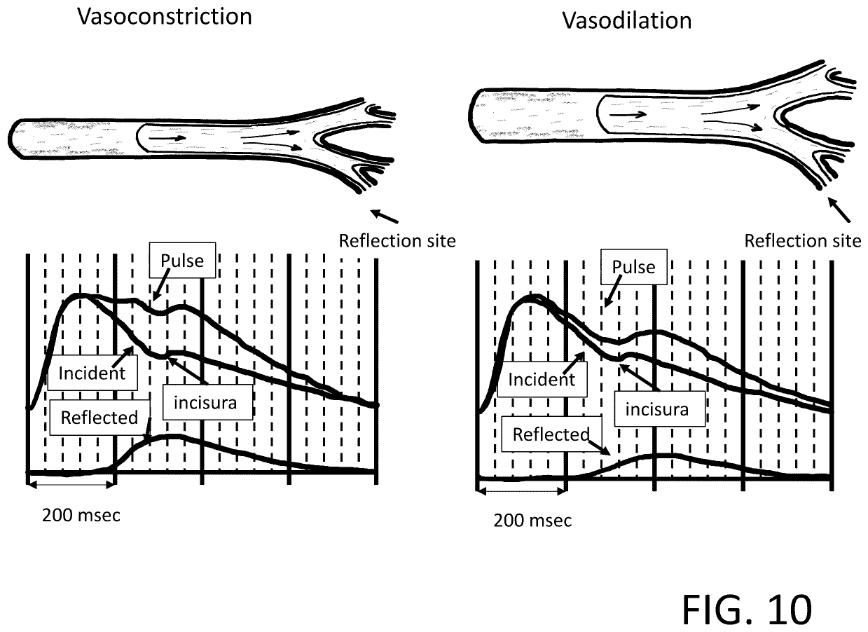

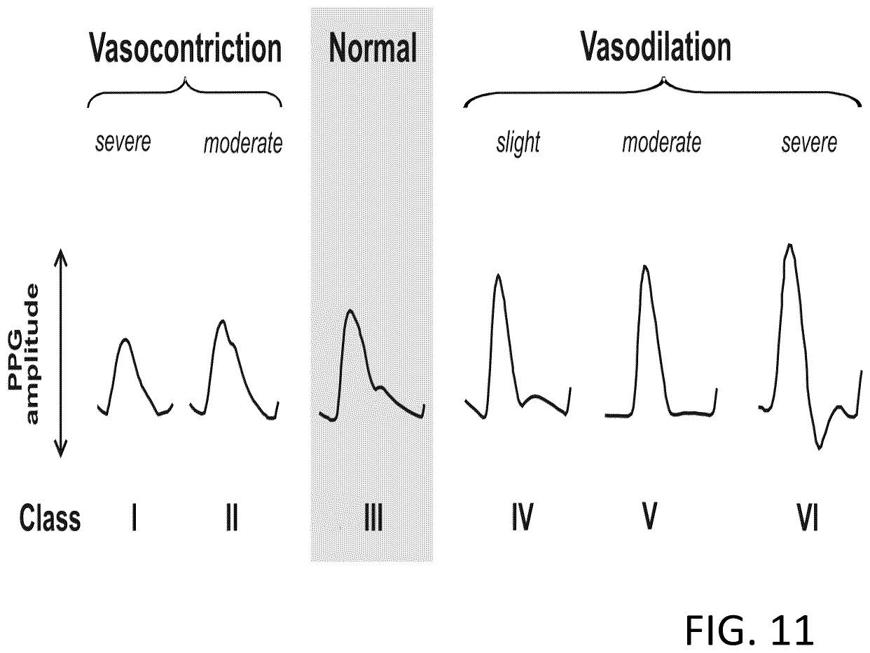

Vascular tone is an important parameter as changes in vascular tone impact the peripheral pulse waveform. Changes in vascular tone impact both the shape of the waveform and also the ability to detect the wave. Peripheral vasoconstriction produces an increase in the amplitude of the reflected wave. Peripheral vasodilation reduces the amplitude of the reflected wave and delays its return, see FIG. 10. In addition to the shape changes illustrated, the size of the pulse wave changes dramatically with the vascular tone, see FIG. 11. Changes in vasodilation create additional variances in the shape of the pulse waveform and are typically more pronounced in the periphery. Thus, the invention seeks to mitigate the measurement impact of changes in vascular tone by hearting the hand or using sampling sites that have diminished changes in vascular tone.

Improved Measurement Systems and methods. Problematic elements of detecting left ventricular outflow tract abnormalities can be addressed by using one or more of the following: (1) improved optical measurement systems, (2) peripheral sampling locations that maximize signal differences, (3) volitional patient maneuvers to improve the diagnostic ability of the system, (4) pulse enhancement techniques, (5) a physiological assessment system configured to analyze the measured systolic time interval information to determine the presence of a repeatable cardiac state; (6) a qualification or validation system configured to indicate the presence of a repeatable cardiac state, and (7) a left ventricular outflow tract assessment system configured to analyze the optical pulse plethysmogram to determine a quantitative assessment of aortic stenosis. In terms of the measurement systems, the in silico simulations demonstrated differences in both flow waveforms and volume (PPG) waveforms. The following section will describe measurement systems and improvements to these systems that enable left ventricular outflow tract abnormality detection.

Flow Measurements. The ability to measure flow in the vascular system has recently been developed using optical speckle measurements. Optical or laser speckle measurements are techniques used to determine blood flow in the body by analyzing the movement of red blood cells within a vessel. These techniques rely on the principles of light scattering and interference. When light is shone on a sample, it is scattered in all directions by the particles within the sample. This scattering is known as speckle, and it can be used to analyze the movement of particles within the sample. In the case of blood flow measurements, the particles being analyzed are red blood cells.

The technique is based on light scattering principles. When a laser beam is directed at a vascular area, the moving red blood cells scatter the light, creating a changing speckle pattern. This pattern, captured by a camera sensor, changes with the flow of blood cells, allowing for blood flow analysis. The analysis of these speckle patterns enables the determination of blood flow velocity and direction in a noninvasive manner. This method is useful for assessing vascular health and diagnosing diseases without the need for direct contact with blood vessels.

Accurate data interpretation to create a speckle plethysmogram relies on algorithms that process the images containing speckle patterns to distinguish between noise, baseline modulations, and the actual movement of blood cells. These algorithms provide reliable blood flow measurements and offer insights into the circulatory system's functioning. This technique stands out for its sensitivity to changes in blood flow, making it an important tool for medical diagnostics and research.

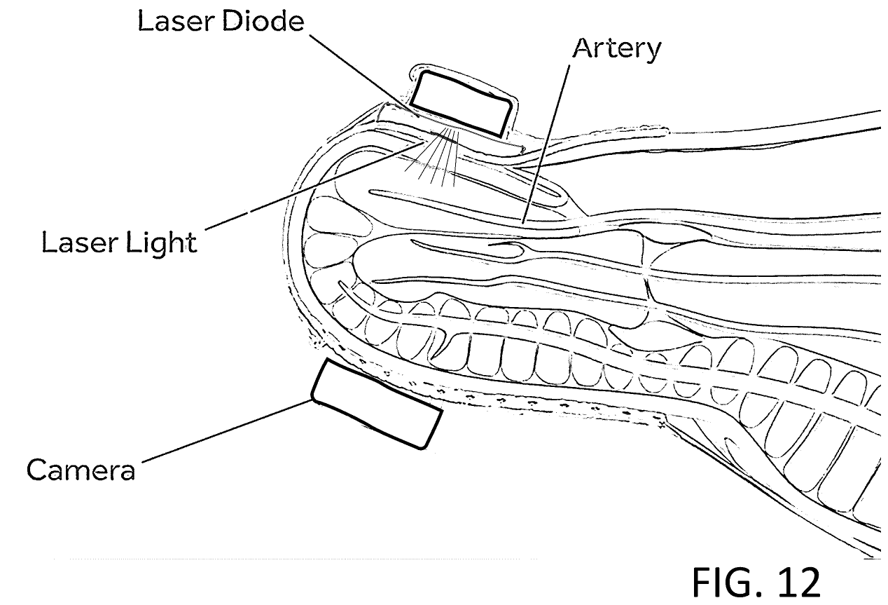

FIG. 12 is an illustration of a speckle based measurement system for use on a finger, where the laser diode and camera sensor are in optical communication with the finger.

There are several different techniques for measuring blood flow using optical or laser speckle, including laser Doppler velocimetry, laser Doppler flowmetry, and laser speckle contrast imaging. These techniques all rely on the same principles of light scattering and interference, but they differ in the specific methods used to measure and analyze the scattered light.

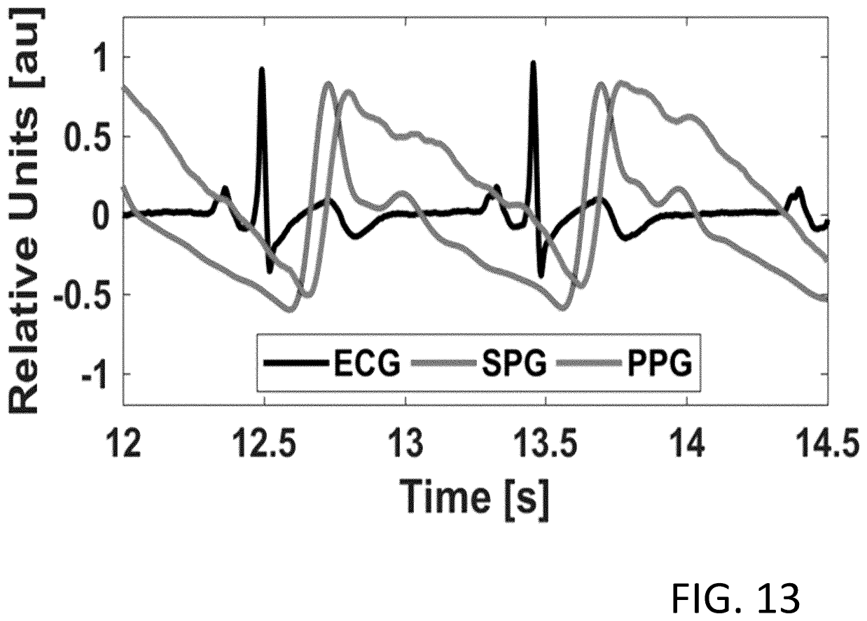

Speckle plethysmography is a flow-based measurement and has some additional benefits relative to photoplethysmography (PPG) in that it is less sensitive to vascular changes and vascular compliance. Dunn et al. state that SPG “provides an improved signal-to-noise ratio and robustness in the presence of motion artifact and cold temperatures as compared to PPG.” (Speckleplethysmographic (SPG) estimation of heart rate variability during an orthostatic challenge.” Scientific Reports 9.1 (2019): 1-9.) The article further states that “similar to PPG, it can be measured from the finger and processed in real-time. In addition, SPG peaks before PPG, which should improve accuracy and reduce the impact of vascular compliance . . . .” The SPG waveform reaches local peaks before the PPG signal, likely due to changes in flow speed (to which SPG is sensitive) preceding expansion and contraction of blood vessels (to which PPG is sensitive). FIG. 13 shows the differences in pulse waveforms between SPG and PPG with an EKG as a reference element. Ghijsen et al. demonstrated reduced sensitivity to vasoconstriction. In a study of volunteers undergoing a cold pressor challenge, the SPG waveform maintained a robust signal-to-noise ratio (SNR) under conditions of significant vasoconstriction relative to PPG, which failed to provide any reading. (M. Ghijsen, T. B. Rice, B. Yang, S. M. White, and B. J. Tromberg, “Wearable speckle plethysmography (SPG) for characterizing microvascular flow and resistance,” Biomed. Opt. Express 9(8), 3937-3952 (2018).) Thus, SPG has four important attributes for the detection of left ventricular outflow tract, (1) higher signal to noise, (2) reduced sensitivity to vascular tine changes, (3) reduced sensitivity to vascular compliance and (4) the measurement of blood flow.

Speckle plethysmography is a noninvasive imaging technique used to measure blood flow dynamics. It relies on the speckle pattern generated by the interference of coherent light scattered from moving red blood cells. The key components or elements of a speckle plethysmography system:

Light Source: A coherent light source, such as a laser, is used to illuminate the tissue of interest. The wavelength of the light source is typically chosen based on the optical properties of the tissue or structure being studied.

Optical Setup: This includes lenses, mirrors, and other optical components to direct and focus the light onto the tissue sample. It also includes the imaging setup to capture the speckle pattern.

Imaging Device: A camera or photodetector is used to capture the speckle pattern formed by the interaction of light with the tissue and the blood cells. The detector should have high sensitivity and spatial resolution to accurately capture the speckle pattern.

Data Acquisition System: This system is responsible for collecting and processing the raw data obtained from the detector. It may include analog-to-digital converters, signal processing units, data storage, and computer interfaces.

Data Analysis Software: Specialized software is used to analyze speckle patterns and extract relevant information such as blood flow velocity, perfusion dynamics, and tissue motion. The software may involve techniques such as temporal or spatial autocorrelation analysis. The software can provide a wide range output, but a typical output is a speckle plethysmogram, which creates a quantitative representation of blood flow in the region of tissue illumination.

Processing System: The processing system controls the speckle plethysmography system and conducts data analysis. It consists of a computer equipped to run the data analysis software.

Speckle plethysmography can be used in several sampling configurations, including transmission sampling through the tissue, reflectance sampling, and imaging modalities. Dunn et al. in a second publication demonstrate that “SPG has a much larger SNR than PPG, which may prove beneficial for noncontact, wide-field optical monitoring of cardiovascular health.” (Cody E. Dunn, Ben Lertsakdadet, Christian Crouzet, Adrian Bahani, and Bernard Choi, “Comparison of speckleplethysmographic (SPG) and photoplethysmographic (PPG) imaging by Monte Carlo simulations and in vivo measurements,” Biomed. Opt. Express 9, 4307-4317 (2018).)

Speckle based measurement methods can be described with different terms, configurations, and capabilities, but all use the principles of light scattering and interference. Laser speckle contrast imaging (LSCI), sometimes referred to as laser speckle flowgraphy or imaging speckle plethysmography, is a noninvasive imaging technique used to measure blood flow in many body locations including the face and retinal. Laser speckle contrast imaging has the ability to determine a pulse signal in distinct retinal vessels over the cardiac cycle. Laser speckle contrast imaging has demonstrated the ability to reliably reveal retinal blood flow dynamics with high spatiotemporal resolution and discriminate between arterial and venous blood flow patterns in the retina. Since retinal blood flow fluctuates over a heartbeat, monitoring the temporal dynamics of retinal vessels creates the ability to procure a pulse plethysmogram that is sampled close to the heart and in an area with exceptional temperature and vascular tone stability.

Li et al. have demonstrated the ability to generate absolute flow speeds using a frequency-domain laser speckle imaging method. “Traditional LSCI (laser speckle contrast imaging) has limited quantitative analysis capabilities due to various factors affecting flow speed evaluation, including illumination intensity, scattering from static tissues, and mathematical complexity of blood flow estimation. Here, we present a frequency-domain laser speckle imaging (FDLSI) method that can directly measure absolute flow speed. (Li, Hao, et al. “Directly measuring absolute flow speed by frequency-domain laser speckle imaging.” Optics express 22.17 (2014): 21079-21087.)

Laser Doppler techniques are based on the optical Doppler effect, which relies on the reflection of a high coherence laser beam scattered in vivo on vascular tissue and captures the shift of the underlying moving red blood cells. The back-scattered light gives a measure of both the incident light (vessel wall) as well as the shifted light (red blood cells), thus providing a measure of relative blood flow, blood volume, and blood velocity within a specified region of the retina. An absolute red blood cell velocity is obtainable by means of bidirectional laser Doppler velocimetry, when the light scattered from the erythrocytes is detected from two directions. For the volumetric blood flow rate calculation, an accurate measure of the diameter is required. Laser Doppler flowmetry does not rely on vessel diameter measurement but is based on the intensity of signal derived from the red blood cell volume and velocity.

Combining the laser Doppler flowmetry with laser scanning tomography, a two-dimensional mapping of retinal blood flow can be obtained, resulting from blood flow measurements based on both single and multiple scattering events from many red blood cells. Local frequency components of the reflected light are obtained at each scanning point and combined with blood velocity. Other Doppler Techniques, including combining OCT with the Doppler technique and laser Doppler holography, have demonstrated flow velocity measurements in many locations.

Optical coherence tomography (OCT) is an imaging technique that uses low-coherence light to capture micrometer-resolution, two-and three-dimensional images from within optical scattering media (e.g., biological tissue). It is used for medical imaging and has been used to access blood flow in the eye.

Volume measurements. PPG, or Photoplethysmography, is a technique used to measure blood volume in human tissue by detecting changes in light absorbance. The measurement is achieved by shining a light onto the tissue and measuring the amount of light that is absorbed. The absorbance of light is directly related to the amount of blood present in the tissue. When there is a higher volume of blood present, the light will be absorbed to a greater extent, resulting in a higher absorbance measurement. Conversely, when there is a lower volume of blood present, the light will be absorbed to a lesser extent, resulting in a lower absorbance measurement. A Photoplethysmography (PPG) measurement is considered volume-based because the measurement is based on absorbance, which measures changes in blood volume rather than the movement of blood itself. In contrast, SPG is based on scatter changes associated with movement of flow of redblood cells, where PPG is based on absorbance changes. A PPG pulse can be simulated effectively by a stationary fluid where the volume sampled by the PPG system is varied. The FingerSim™ produced by BC Biomedical uses this concept (BC Group International Inc., 3081 Elm Point Industrial Dr., St. Charles, MO 63301-4333). As stated in their manual, “The time varying light absorption component required by a pulse oximeter is created in the FingerSim™ by rhythmically pressing the color coded end. This creates a volume change in the distal (sensor) end of the FingerSim™, analogous to the heart creating blood pressure waves that force blood into the finger. The amplitude and rate of the pulse wave can be varied by changing the applied pressure and interval.” PPG allows for the continuous measurement of blood volume in human tissue by measuring the absorbance of light at regular intervals. The method can be used to measure changes in blood volume resulting from cardiac function.

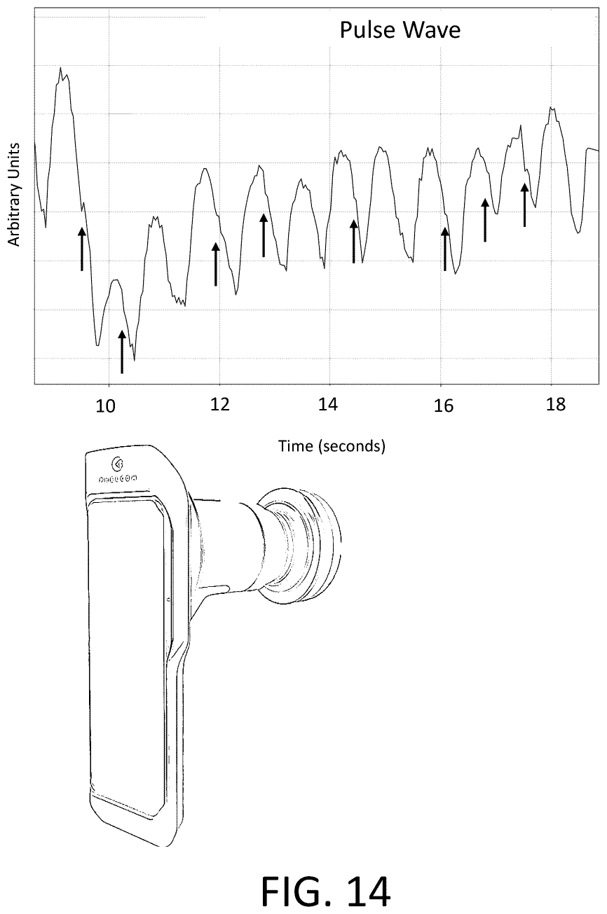

Many different implementations of the basic concept are within the scope of the invention, including transmission sampling, reflection sampling and imaging modalities. Imaging photoplethysmography can be used to obtain pulse waveforms from many locations on the human body, including the hand, face, eyelids, and the eye. Imaging photoplethysmogram (iPPG) is a remote and non-contact alternative to the conventional PPG in humans. iPPG is typically acquired using a video camera under dedicated or ambient light. The video is typically recorded from palm or face regions. The technique has also been used to assess retinal blood flow. FIG. 14 is an example of an eye imaging PPG system using a mobile phone and an example of the resulting photoplethysmography. Video images were obtained with a handheld, non-mydriatic fundus camera. The resulting video was processed by amplifying the pulse signal using RGB color panels and processing a region of interest around the optic nerve. The resulting signal was high pass filtered, and eye blinks were removed. The resulting signal for a person with no left ventricular outflow tract abnormality shows a pulse with a clear dicrotic notch, identified by the arrows. A variety of camera and optical systems can be used to obtain an image-based photoplethysmography by capturing a series of fundoscopic images or a video that enables subsequent processing for determining a pulse plethysmogram. A standard fundoscopic camera that can take repeat images at an adequate sampling rate enables the creation of a pulse waveform that can be used to screen for left ventricular outflow tract abnormalities. Morgan, William H., et al. have also shown pulse measurements for the eye. (Photoplethysmographic measurement of various retinal vascular pulsation parameters and measurement of the venous phase delay. Investigative ophthalmology & visual science 55.9 (2014): 5998-7007. Tornow et. Al.) have “presented low-cost video-ophthalmoscope permits measurement of the plethysmographic signal of the ONH (optic nerve head) tissue and calculation of different blood flow-related parameter.” (Tornow, Ralf-Peter, et al. “Imaging video plethysmography shows reduced signal amplitude in glaucoma patients in the area of the microvascular tissue of the optic nerve head. Graefe's Archive for Clinical and Experimental Ophthalmology 259.2 (2021): 483-494.)

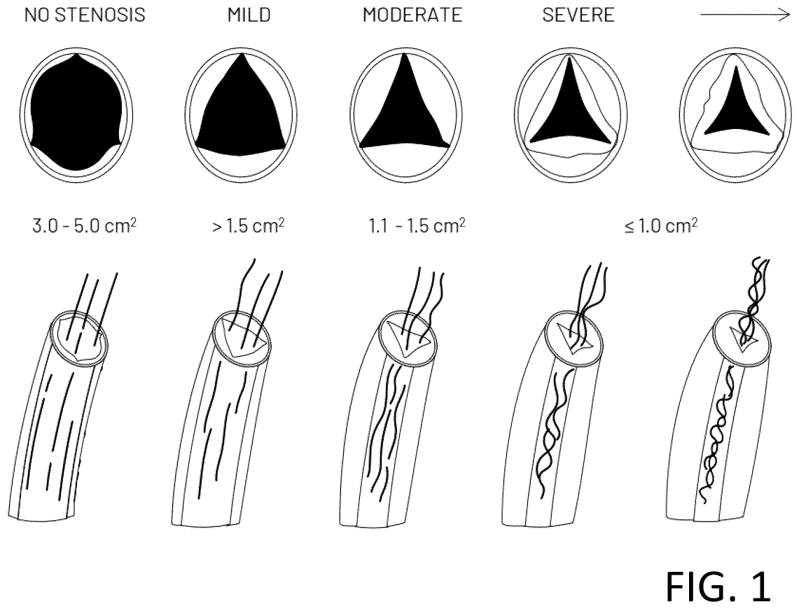

The process can be used at the individual vessel level as shown in FIG. 1 of Tornow's publication or based upon the entire image. The pulse waveform can also be calculated from the pulsatile changing light reflection due to the pulsatile changing amount of blood in the retinal vessels as well as changes in the location of various tissue types. The use of larger regions of interest for optical processing requires less stability of the measurement platform as it is not particular for a specific artery segment.

An additional system with improved measurement capabilities includes the ability to measure arterial waveforms in both eyes concurrently. Because aortic stenosis often creates peak velocity jets and there is an asymmetry in the vascular access to the eyes, the concurrent measurement of valvular pulses can provide additional diagnostic information. The parallel video acquisition of both eyes simultaneously can enable the comparison of PPG signals between both eyes with a high temporal and spatial resolution for the detection or deviation from symmetry. Tornow et al. have demonstrated that differences in eye pulse asymmetries can occur in the presence of carotid artery stenosis. (Tornow, Ralf-Peter, Jan Odstrcilik, and Radim Kolar. “Time-resolved quantitative inter-eye comparison of cardiac cycle-induced blood volume changes in the human retina.” Biomedical Optics Express 9.12 (2018): 7237-7254.)

A system for obtaining such images can use a headset similar to a virtual reality headset such that the patient can focus on a moderately dark screen with limited eye movement. The use of a moderately dark screen facilitates pupil dilation. Additionally, instructions associated with cardiac maneuvers can be supplied to the patient. A funduscopic imaging system in the headset enables the acquisition of fundoscopic images over time as the patient views the screen.

Multiple processing methods are suitable for the determination of pulse waveforms for the eye. These methods can use a single location, a single vessel, a region of interest, or the entire image. Additionally, these data can be acquired with no pressure applied to the eye or a small increase in intraocular pressure to further facilitate a decrease in arterial transmural pressure and reduce contributions from venous sources.

### Sampling Location for Left Ventricular Outflow Tract Abnormality Detection

Sampling locations that maximize the waveform differences between normal physiology and that present with left ventricular outflow tract abnormalities is desired. Additionally, locations that minimize between-patient pulse wave transformation differences as well as within patient pulse wave transformation differences are desired.

Radial or Ulnar Artery Sampling: the radial and ulnar artery are the two main arteries supplying blood to the hand. These arteries are easily accessed for sampling. Importantly, the wrist area, especially the volar aspect (the inner part of the wrist), is not typically an area of high weight gain. Unlike areas such as the abdomen, hips, thighs, and upper arms, the wrist is composed of less adipose (fat) tissue and is primarily made up of bones, tendons, vascular elements, and ligaments. The structure of the wrist is designed for movement and stability rather than fat storage. Thus, this peripheral sampling location allows access to a significant artery and represents a sampling area that will be less sensitive to high BMI patients versus auscultation of the abdomen.

Carotid Artery Sampling: The carotid arteries represent a desired peripheral sampling location for optical measurements due to their accessible location, proximity to the heart, and their reflection of central blood flow dynamics. Positioned superficially in the neck, they allow for a noninvasive, straightforward assessment, eliminating the need for invasive procedures. Their closeness to the heart ensures that flow measurements undergo limited distortion due to the distance traveled. The optical plethysmogram from these arteries will closely resemble aortic dynamics. Thus, the carotid arteries offer a practical and effective site for obtaining information for the assessment of left ventricular outflow abnormalities and the assessment of the degree of aortic stenosis.

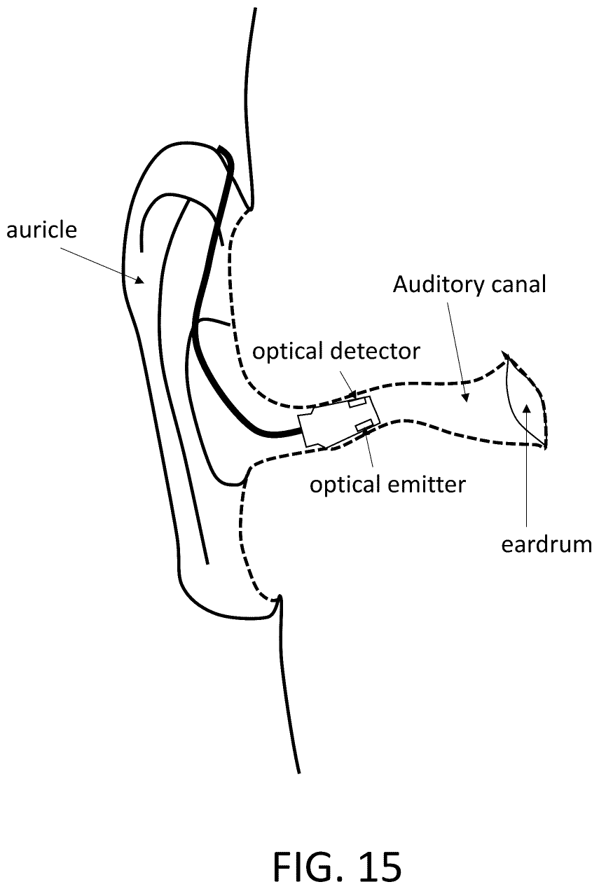

Ear canal sampling. The ear, specifically the auditory canal, is a desirable sampling location due to its proximity to the heart, consistent perfusion with minimal changes in vascular tone, and decreased transmural pressure when standing or sitting. FIG. 15 illustrates the sampling location in the auditory canal where the sensor and detector are in optical communication with the tissue. Additionally, waveform differences resulting from reflections were evident in the in silico simulations.

Eye Sampling. The retinal vessels in the eye represent another sampling location that exhibits consistent perfusion with reduced changes to local vascular tone and decreased transmural pressure when the patient is sitting or standing. Importantly, the ability to access the arteries and arterioles of the eye via an “optical window” has a multitude of advantages. Other sampling locations on the body necessitate measurement through the skin.

Chest. The chest represents a unique sampling location due to the minimal distance traveled in the arterial vasculature. Additionally, the procurement of ancillary signals including electrocardiogram, phonocardiogram, seismocardiogram, and ballistocardiography are easily obtained from the chest.

Distal Finger, the distal finger represents another sampling location due the rich capillary network present in the finger and the ability to do either transmission or reflectance sampling.

Distal Arteries, The digital arteries of the finger are small blood vessels that supply blood to the fingers, branching from the palmar and dorsal arteries of the hand. Each finger is supplied by two arteries. The sampling location supports both reflectance and transmission sampling.

The above sampling locations have the following three characteristics: (1) reduced hydrostatic pressure, (2) reduced vasodilation/vasoconstriction relative to the hand, and (3) moderately close to the heart.

### Additional Diagnostic Information

Volitional patient maneuvers to improve the diagnostic ability of the system. Different left ventricular tract obstructions and valve abnormalities can produce pulse waveforms that have similar characteristics. The ability to determine the location and type of abnormality can be enhanced by using cardiac maneuvers. Cardiac maneuvers are volitional actions performed by the patient that modify cardiac function in a deterministic fashion by changing filling pressure, afterload pressure, or heart rate.

For example, the Valsalva maneuver has been used during dynamic auscultation to help distinguish different cardiac conditions. The murmur of aortic stenosis typically increases with maneuvers that increase left ventricular volume and contractility (e.g., leg-raising, squatting, Valsalva release, after a ventricular premature beat) and decreases with maneuvers that decrease left ventricular volume (Valsalva maneuver) or increase afterload (isometric handgrip). These dynamic maneuvers have the opposite effect on the murmur of hypertrophic cardiomyopathy, which can otherwise resemble aortic stenosis. The murmur of mitral regurgitation due to prolapse of the posterior leaflet may also mimic arterial stenosis. Maneuvers can be used to change the filing pressure into the hearts and the afterload in a deterministic fashion to create additional diagnostic information based on the pulse waveform.

The pulse waveform will change in the diseased and normal heart as the patient performs certain maneuvers. The change in the pulse waveform can be used as a clinical tool during testing to aid in diagnosing the specific heart abnormality. The following maneuvers create deterministic changes impacting cardiac function. The type of preload or afterload change is specified with each maneuver and the typical impact on heart murmur intensity is included.

Handgrip: Increases afterload of blood pressure. Hand gripping increases the strength of aortic regurgitation, mitral regurgitation, and ventricular septal defect murmurs. It decreases the intensity of murmurs due to hypertrophic obstructive cardiomyopathy and mitral valve prolapse. In aortic stenosis patients, the murmur decreases slightly due to the decrease in the pressure gradient.

Squatting: Increases blood flow to the heart, resulting in increased preload. Squatting increases the intensity of aortic stenosis, mitral stenosis, aortic regurgitation, and mitral regurgitation. It decreases the strength of murmurs due to hypertrophic obstructive cardiomyopathy and mitral valve prolapse.

Valsalva: Decreases blood flow to the heart resulting in decreased preload. Valsalva increases the strength of murmurs due to hypertrophic obstructive cardiomyopathy and mitral valve prolapse. It decreases the intensity of aortic stenosis, mitral stenosis, aortic regurgitation, mitral regurgitation, and ventricular septal defects.

Standing abruptly: Decreases preload and has the same effects as Valsalva. Sudden standing increases the intensity of murmurs in hypertrophic obstructive cardiomyopathy and mitral valve prolapse. It decreases the strength of aortic stenosis, mitral stenosis, aortic regurgitation, mitral regurgitation, and ventricular septal defects.

Resistance breathing, paced breathing, or controlled breathing can be used to create deterministic changes in cardiac function. Changes in intrathoracic pressure influence the venous flow to the heart. For example, high intrathoracic pressure obtained during exhalation against resistance will decrease the venous return to the heart. Control of the rate and degree of resistance on either inhale or exhale or both creates repeatable changes to cardiac function that can be used to facilitate the detection and diagnosis of left ventricular tract obstructions.

A resistance breathing device is used by the patient and is used to create systematic airway pressure changes. A resistance breathing device can be used to create exhalation resistance to create an abnormal increase in intrathoracic pressure during exhalation. Correspondingly such a device can create inhalation resistance resulting in an abnormal decrease in intrathoracic pressures. Additionally, the system can use different levels of resistance over the course of the protocol. Multiple methods of implementation exist for altering intrathoracic pressure above normal levels. Resistance breathing devices can include the use of pressure threshold, flow-independent pressure valve, air restriction mechanisms, and any mechanism that causes an increase in pressure during normal breathing. Additionally, the term resistance breathing covers the process of creating a change in intrathoracic pressure where little or no air movement occurs for a period of time. The creation of an occlusion pressure to increase or decrease intrathoracic pressure is encompassed as part of the broad definition of resistance breathing.

Continuous positive airway pressure (CPAP) is a form of resistance breathing where the system maintains a constant positive pressure throughout the breathing cycle. Continuous positive airway pressure increases intrathoracic pressure, thereby decreasing the venous filling pressure or preload to the heart. CPAP can be used to create a systematic change in the filling pressure to facilitate the location and type of abnormality, as well as the severity of the abnormality. In practice, many CPAP machines have a ramp mode that increases the CPAP pressure in a systematic and controlled fashion. This results in a systematic and controlled variance in the filling pressure to the heart.

Paced breathing applies to any method that defines a breathing rate and can include depth of breathing. Paced breathing is typically slow at a rate between 5 and 7 breaths per minute. With normal breathing, the rate is about 12 to 14 breaths a minute. Paced breathing can include defined changes in the rate and an asymmetric breathing profile; for example, the exhale is 8 seconds while the inhale is 5 seconds.

Controlled breathing is the process of combining resistance and paced breathing to influence cardiac changes. The “controlled” aspect is a system or method of breathing that dictates breathing rate and creates an intrathoracic pressure change that is greater than normal breathing. Examples of controlled breathing include but are not limited to a mini-Mueller inhale against resistance using a pressure threshold, but flow independent value followed by a mini-Valsalva against resistance using a pressure threshold but flow independent value at a rate of 7 breaths per minute.

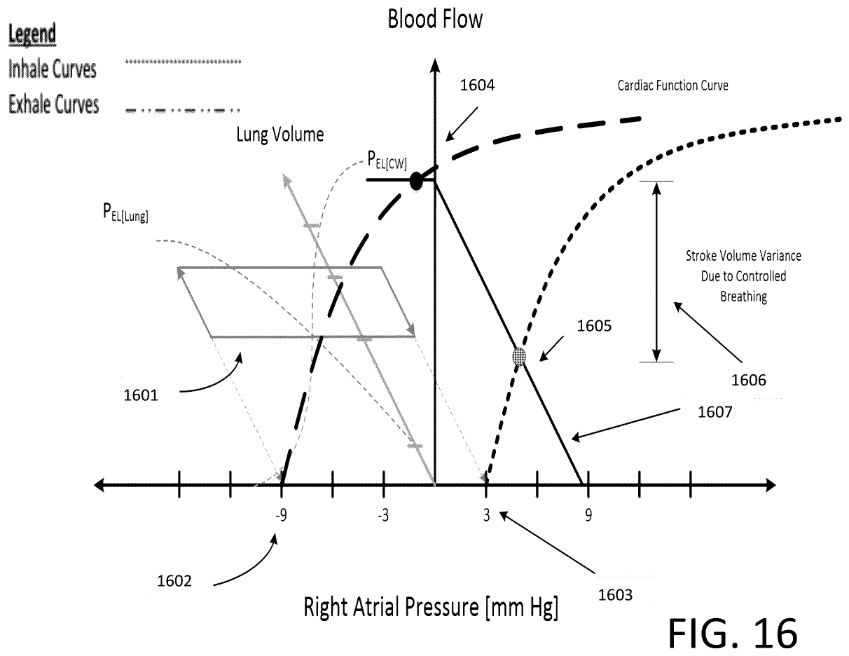

The value of a controlled breathing process can be well illustrated through the Combined Heart-Lung diagram. Variance due to controlled breathing in this process is diagrammed in FIG. 16 with a −7 and +7 mm Hg controlled breathing protocol. Note the “flat” or “box” portion of the Campbell diagram shows the influence of the resistance threshold system. The pressure increases with little change in lung volume until the threshold of the device is obtained. The device then maintains a moderately constant pressure until the exhale or inhale is completed, see 1601 as an example of “flat portion” of inhalation. Note also the large left shift of the cardiac function curve with inhalation, 1602, and the opposite right shift of the cardiac function curve with exhalation, 1603. These changes impact the cardiac operating points as shown in 1604 for inhale and 1605 for exhale. The resulting cardiac operating points cause a large change in the cardiac output or stroke volume. The change is identified by arrow 1607 which shows the difference in cardiac output between the inhale and exhale. 1607 shows the venous return curve in this schematic.

The above mini-Mueller and mini-Valsalva controlled breathing system can be configured so that pressures are the same on inhalation and exhalation (symmetric) or different on inhalation and exhalation (asymmetric). Note that the resistance pressure can be modified to facilitate different defined intrathoracic pressure changes. The resistance pressures can magnify normal changes in intrathoracic pressure leading to more significant changes in venous return, thus effectively creating an improved signal-to-noise measurement. These larger than normal physiological changes in venous return subsequently create larger changes in stroke volume and facilitate hemodynamic assessment.

Controlled breathing, typically at six breaths per minute, can be implemented at zero resistance or multiple defined levels. A significant benefit of a controlled breathing protocol at different resistance levels is the creation of a moderately consistent breathing process with multiple levels of evaluation. In testing the system, we have observed that some patients expand their chest while others use a more abdominal breathing mechanism. Kimura et al. demonstrated that changes in inspiration between diaphragmatic versus chest wall expansion influenced inferior vena cava diameter and would thus influence the venous return curve. Kimura, Bruce J., et al. “The effect of breathing manner on inferior vena cava diameter.” European Heart Journal-Cardiovascular Imaging 12.2 (2011): 120-123. The present invention can reduce the influence of subject breathing type by using the information at two different pressure levels to help normalize subject-specific breathing differences. Additionally, as shown in the Campbell diagram previously, changes in lung volume interact with lung and chest wall compliance. Therefore, dramatic changes in tidal volume will have a direct impact on intrathoracic pressure. Therefore, a benefit of the controlled breathing system is to create repeatable, defined intrathoracic pressure changes where tidal volume differences are minimized.

Exercise can also be used to induce changes in cardiac function for additional diagnostic capabilities. Exercise creates various physiological changes, including increased heart rate, heart contractility, decreased systemic vascular resistance, and increased blood pressure.

Pharmaceutical agents can also be used as part of the diagnostic approach. Amyl nitrate is a drug that decreases afterload. Amyl nitrate increases the intensity of aortic stenosis, hypertrophic obstructive cardiomyopathy, and mitral valve prolapse. It decreases the severity of aortic regurgitation, mitral regurgitation, and ventricular septal defects.

Other types and forms of patient maneuvers can modify cardiac performance in a deterministic fashion. These changes create alterations in the pulse waveform that further the diagnostic resolution of the system. These maneuvers provide additional information that enhances the system's screening and diagnostic capabilities. The maneuvers are implemented to create repeatable perturbations to cardiac function.

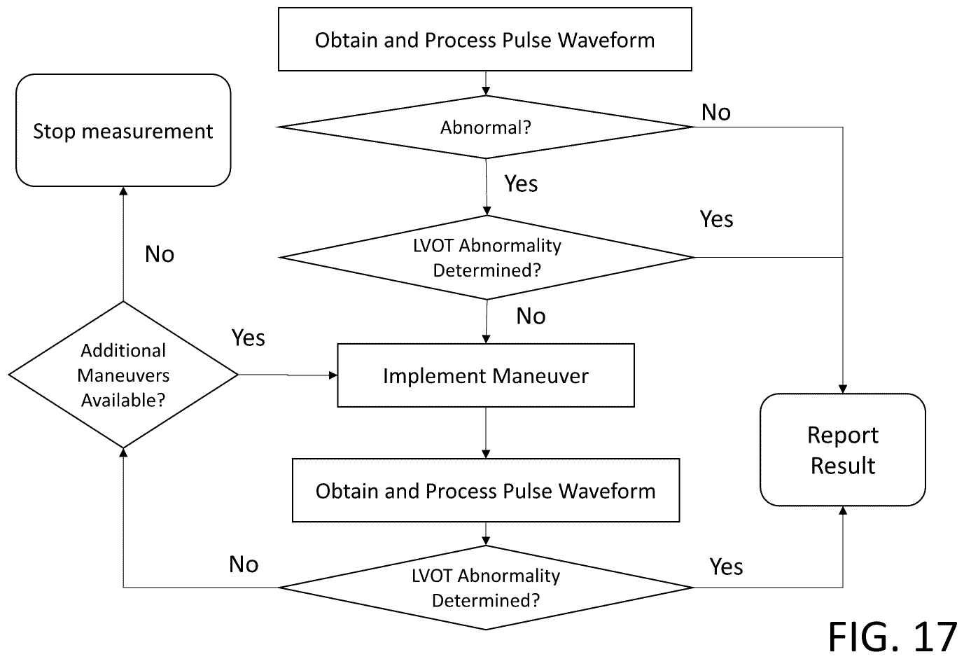

FIG. 17 illustrates a method where cardiac maneuvers are used to improve the diagnostic resolution of the system to identify the specific left ventricular outflow tract abnormality.

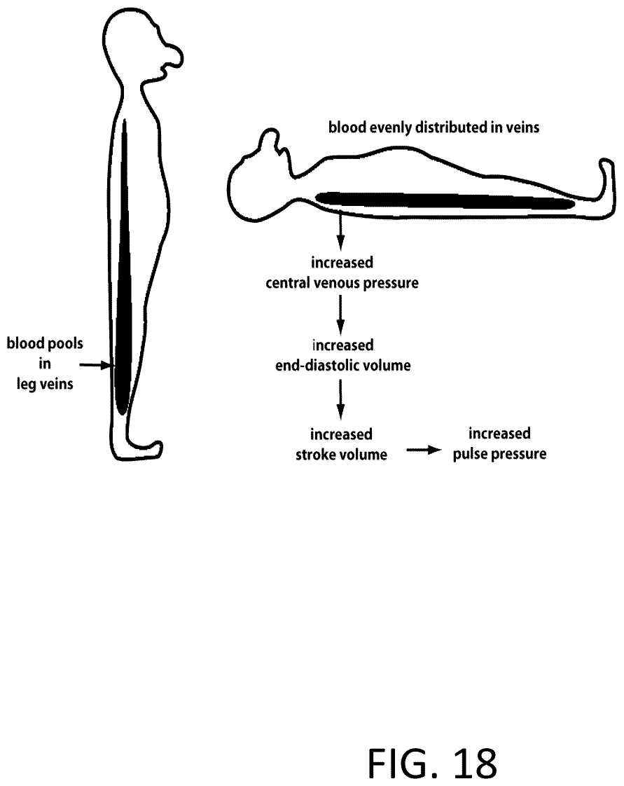

Positional changes are a type of volitional change that increases the filling pressure to the heart as the patient moves between standing, sitting, and supine postures, as well as change in leg position relative to the thorax. Movement from the standing position to the supine position results in the translocation of approximately 300 ml to 500 ml from the lower extremities towards the intrathoracic vessels and produces an increase in venous return and cardiac preload. According to the Frank-Starling law, there is a positive relationship between preload and systolic volume; accordingly, the greater the ventricular preload (and therefore the degree of cardiac muscle stretch), the greater the systolic volume ejected. Positional changes and their impact on cardiac function are illustrated in FIG. 18.

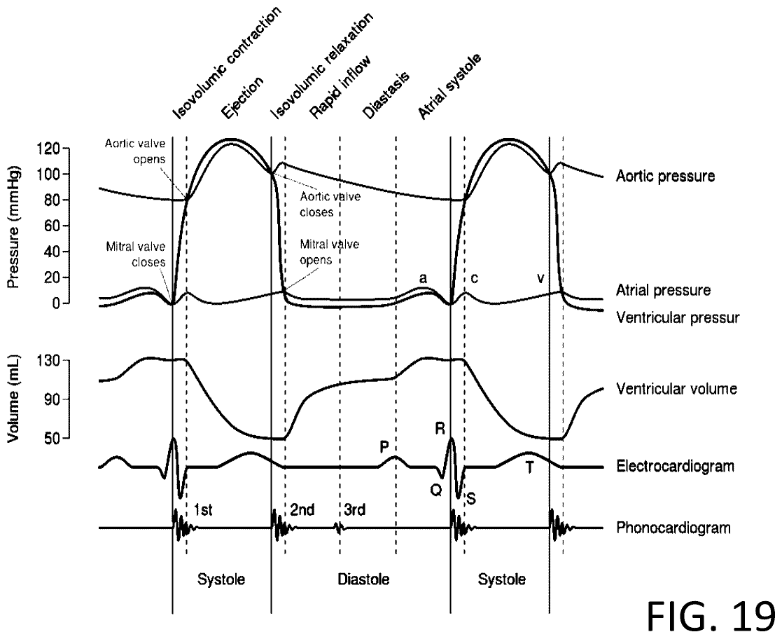

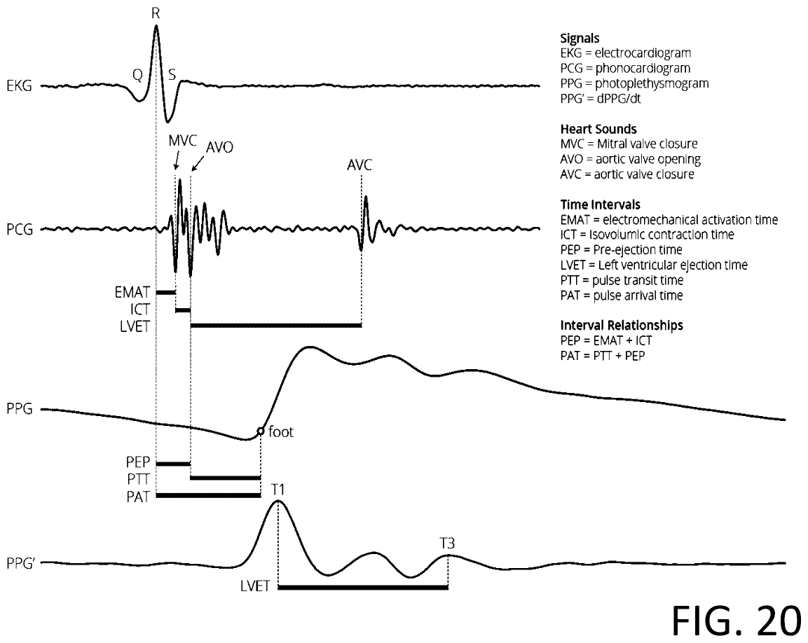

Ancillary Signals. Ancillary signals pertain to measurable signals associated with cardiac or respiratory functions. Determining left ventricular outflow tract obstructions can be enhanced through ancillary signals such as an electrocardiogram (EKG), phonocardiogram, or seismocardiogram. In the EKG, variances in T-wave and QT interval have shown some correlation to aortic stenosis. EKG measurement also enables the determination of pulse transit time, which has shown a correlation with arterial stiffness and functional properties of the arterial tree. Phonocardiography, the recording of all the sounds made by the heart during a cardiac cycle, and seismocardiography, the recording of body vibrations induced by the heart, can also be used for data augmentation. These measurements contain information regarding cardiac mechanics and the timing and nature of valvular transitions. FIG. 19 shows a pressure, volume, and timing diagram of the cardiac cycle, as well as EKG and phonocardiogram signals. FIG. 20 shows the relationships between certain measured parameters and the ECK, PCG, and PPG signals and serves as reference for a variety of determined time intervals.

Ancillary Information. Including vital signs, anatomical and functional information, physiological, and demographic information can help with the evaluation process. Potential ancillary information can include blood pressure, heart rate, stroke volume, left ventricular ejection time, age, gender, health status, height, weight, medications, mean arterial pressure, pulse wave velocity, and arterial diameter. This ancillary information can provide important information associated with understanding the type and degree of pulse waveform transformation expected for a similar individual with a ventricular outflow tract obstruction. As shown in FIG. 9, age is a factor in accessing the morphology of the waveform. Another important factor is blood pressure, which influences pulse wave velocity and impacts forward and reflected wave propagation.

### Pulse Enhancements

Given the subtilities of the information present in the pulse waveform and the fact that the patient might have a degree of cardiovascular compromise, improving the size of the pulse signal is desirable. The size of arterial pulsations can be increased by decreasing the vascular transmural pressure (TMP), that is, the pressure gradient across artery walls. At least three mechanisms are responsible for this enhancement in pulse size with TMP decrease: (1) decreases in TMP trigger arterial dilations through the local venoarterial reflex (VAR), (2) decreases in TMP trigger the myogenic response, i.e., the relaxation of the smooth muscles in artery walls, and (3) because vessel compliance is a function of TMP, decreases in TMP increase arterial compliance such that a given change in arterial pressure results in a larger change in arterial volume. TMP can be reduced by applying external pressure at the measurement site or raising the elevation of the measurement site relative to the heart to decrease hydrostatic pressure.

External pressure pulse enhancement. The application of external pressure at the sampling sight can be used to reduce the TMP. The optimal external pressure is typically greater than the venous pressure but less than the arterial diastolic pressure; pressures beyond this point will begin to occlude flow and distort the pulse waveform. Based on the work of Balijepalli et al. (2014), 95% of individuals aged 18-99 years have a diastolic pressure above 70 mmHg. If the sampling site is near or below the level of the heart, external pressures in the range of 50 mmHg can be appropriate to increase the magnitude of arterial pulsation.

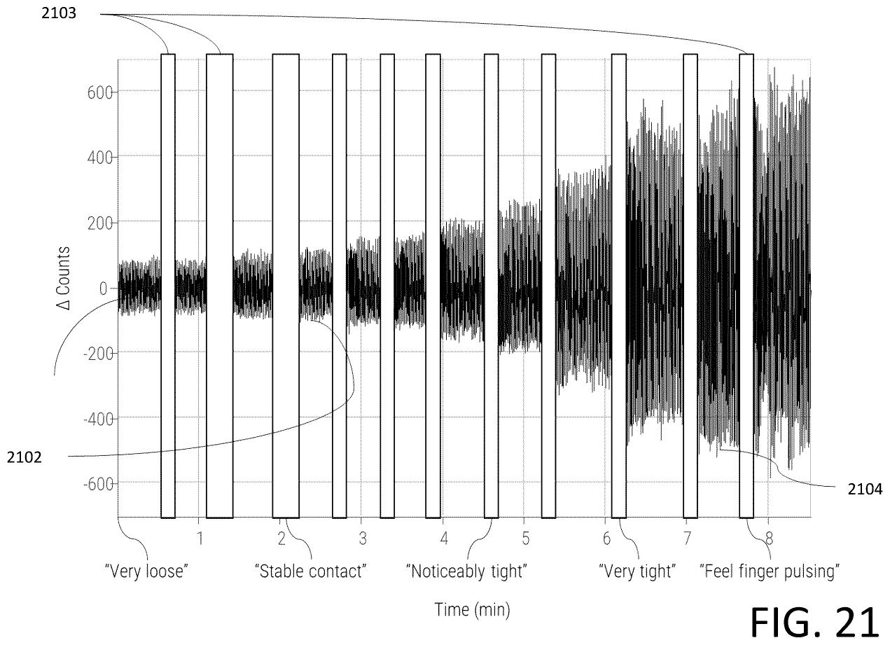

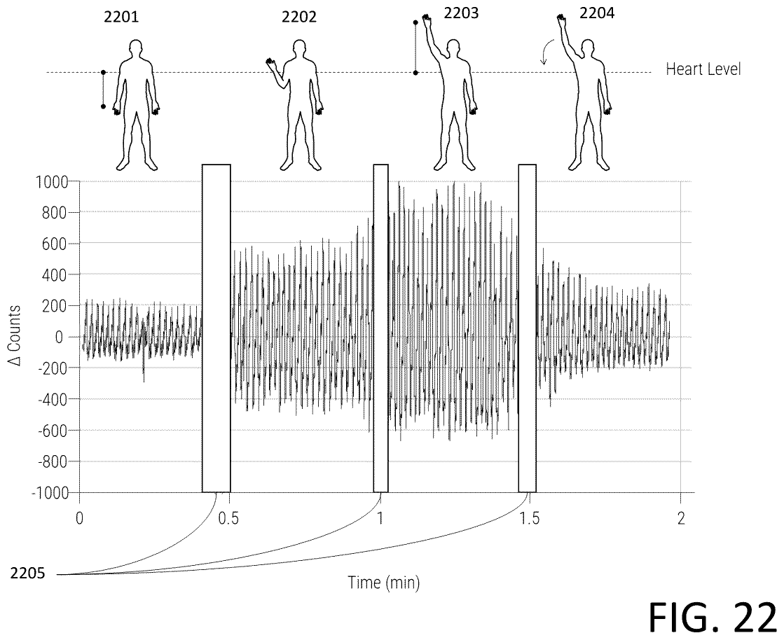

As shown in FIG. 21 and FIG. 22, the effect of TMP on pulse size is graded. Thus, any appreciable external pressure (e.g., greater than 5 mmHg) will produce some increase in the pulse. (Balijepalli, C., Lösch, C., Bramlage, P., Erbel, R., Humphries, K. H., Jöckel, K.-H., & Moebus, S. (2014). Percentile distribution of blood pressure readings in 35783 men and women aged 18 to 99 years. Journal of Human Hypertension, 28(3), 193-200.)

FIG. 21 shows the detector signal from an adjustable PPG ring worn at the base of the finger. The signal has been band-pass filtered to focus on the pulsatile component. Roughly every 45 s, the ring is tightened incrementally on the wearer's finger via a ratcheting mechanism on the ring band. These tightening events are denoted by white rectangles, 2103. The wearer's reported subjective experience associated with the different levels of tightness is indicated below the graph. Initially, in period 2101, the ring is reported by the user to be “very loose,” and the magnitude of the pulse is ˜100 detector counts. After several tightening events, the user reports that the ring makes “stable contact” with the finger. The pulse size at this period (2102) is ˜150 counts. After this point, each tightening event increasingly changes the TMP through the applied external pressure, as evidenced by the increase in pulsatile signal size. When the ring is reported by the user to be “very tight,” the pulse size increases to ˜1000 counts (period 2104). After further tightening, the user reports feeling pulsations in the finger, an indication that the external pressure is approaching arterial diastolic pressure. Cumulatively, the tightening events produced a 10% reduction in the circumference of the ring and created a 10-fold increase in signal size is due to the decrease in arterial TMP caused by the increased external pressure at the sampling site.

The measurement device includes a system and mechanism for decreasing the transmural pressure (TMP) for maximal signal quality by adjusting the TMP based on patient physiology. The system allows patient-specific adjustments to the TMP that compensate for anatomical differences, blood pressure differences, and other patient-specific nuances. Adjustments can be manual or automatic in nature, facilitating the procurement of high-quality pulse waveforms by increasing the TMP to a pressure that does not compromise arterial flow. It is important to note that the external pressure resulting in a maximal pulse waveform is not desired for long-term monitoring as the patient may report feeling pulsations in the finger, which may be slightly disconcerting. As the device is a designated test over a limited period, this discomfort is viewed as acceptable. The force exerted by a typical fingertip oximeter is commonly less than optimal due to potential discomfort of the user as they are used for longer term monitoring, and the spring used to hold the device on the finger is not configurable based on patient specific characteristics.

Hydrostatic pressure pulse enhancement. The reduction of internal arterial hydrostatic pressure at the sampling sight can be used to reduce the TMP. FIG. 22 shows a second example of the effect of TMP on pulse size, in this case, using manipulations in hydrostatic pressure to alter the TMP. FIG. 22 shows a band-pass filtered detector signal from a PPG ring worn at the base of the finger. The ring size is constant throughout the experiment, but the subject undergoes changes in arm positions, indicated by white rectangles 2205. In period 2201, the arm hangs in a relaxed position at the subject's side. The sampling site is estimated to be 50 cm below the right atrium of the heart, resulting in ˜37 mmHg of additive pressure distending the walls of the veins and arteries due to the hydrostatic pressure exerted by the vertical columns of blood in these vessels. The pulse size in this period is just under 400 counts. In period 2202, the subject raises their hand such that the sampling site is roughly level with the shoulder. The change in vertical displacement with respect to the heart decreases the hydrostatic pressure, decreasing the TMP accordingly. The pulse size therefore increases by more than a factor of 2 to nearly 1000 counts. In period 2203 the subject extends their arm to a comfortable position above their head. The sampling site is now an estimated 77 cm above the right atrium, resulting in hydrostatic pressure of roughly-50 mmHg. This height reduces the TMP, which causes a further increase in the pulse size to roughly 1500 counts. In period 2204, the subject slowly lowers their arm down. As would be expected, the pulse size gradually decreases.

Decreasing TMP at the sampling site provides the additional benefit of reducing physiological signals that are unrelated to the arterial pulse waveform. A large source of physiological noise is venous blood. Since the venous system operates at relatively low pressures, it is quite susceptible to the local effects of volume perturbation during motion. The venous blood in the vascular bed will be easily deformed during subtle motion, changing light absorption, and producing a significant source of in-band noise. This noise source can be managed by reducing the venous TMP to below zero, effectively collapsing the veins to minimize their volume. This process stabilizes the venous contribution to the vascular volume and reduces the overall absorbance of light by non-pulsatile sources. The ability to increase the size of the pulse has benefits for all optical measurement methods.

Vasodilation by heating. Heating the hand can cause vasodilation, which is the widening of blood vessels. When blood vessels dilate, more blood can flow through them, which leads to an increase in pulse amplitude. This increase in pulse amplitude is due to the increased blood flow and volume in the blood vessels. When tissues are exposed to heat, the blood vessels near the surface of the skin dilate to help regulate body temperature. As the blood vessels widen, more blood flows through them, leading to an increase in pulse amplitude.

Optical sampling design. The physical configuration of light emitters and detectors in an optical system also plays an important role in determining the optical path length and the type of vessels sampled. When the emitters and detectors are placed in close proximity (e.g., separated by <5 mm) or with little angular difference (less than 10 degrees) the detected photons are more likely to have interacted primarily with superficial vessels in the capillary bed. When the detector is at a greater separation or greater angle from the emitters, the photons that reach the detector are more likely to have interacted with deeper tissue containing more proximal arterial segments. For detecting structural and valvular abnormalities, the system seeks to maximize the SNR related to flow abnormalities or pulse waveform abnormalities by a deeper sampling of larger vessels such as arteries and arterioles.

The apparatus used for auditory canal sampling can be inserted into the ear canal, and the relationship (distance and angle) between the detector and emitter is optimized for measuring information-rich and maximized SNR pulse waveforms. The apparatus may also be configured to fit snugly within the auditory canal or expand after insertion. The application of mild positive pressure on the inside of the auditory enables stable contact of optical components and decreases transmural pressure across the vessels of interest.

Supine Position. To assess a left ventricular outflow tract abnormality, maximizing the amount of blood ejected per beat is beneficial. The impact of a decrease in the cross-sectional area of the aortic valve can be minimal at low stroke volumes but will increase as the amount of blood that must pass through the valve with each beat increases. Placement of the patient in the supine position maximized stroke volume and is a method for maximizing pulse size. As the patient is stationary during this activity, it was not viewed as a volitional activity but rather a pulse enhancement activity. For the purpose of repeatable testing, the patient should be in a preload independent state. Preload independence defines a physiological state where the variations in cardiac filling pressures have minimal effect on stroke volume. Preload independence occurs during conditions of high venous return when the heart is filled at or around natural capacity. The location of the body in a supine position facilitates preload independence by increasing venous return.

Sensor Control System. Sensor control system controls the operation of the sensor system and ensures that an adequate measurement signal is obtained. Sensor control system will also acquire ancillary information and ancillary signals as necessitated by the measurement protocol. Additionally, the sensor control system may perform quality control on the measurement signal and optimization of the sensor system for optimal performance by altering the operational parameters. Operational parameters can include the optimization of light intensity, optimization of transmural pressure decrease, changing the optical focus in imaging situations, determining the region of interest in imaging applications, changing the duration of data acquisition, and the sampling rate of data acquisition. Additionally, the sensor control system can ensure that volitional activities are completed and correctly executed via assessment of the measures signal or the use of ancillary signals.

### Reducing Optical Sampling Noise with Index-Matching Fluid

When sampling skin with an optical sensor, using an index-matching fluid between the sensor and the skin is a technique to improve the quality of the measurements. This approach has two primary benefits: it reduces reflections from the skin surface and reduces optical noise when there is movement of the sensor relative to the skin.

The skin surface acts as a partially reflective barrier for light due to the difference in the refractive index between air (approximately 1.0) and skin (approximately 1.33 to 1.4 depending on the exact tissue). When light from an optical sensor strikes the skin surface, a significant portion of it can reflect away, leading to less light penetrating into the skin where it can interact with the tissues and provide meaningful data. By using an index-matching fluid, which has a refractive index close to that of skin, the disparity between the refractive indices of the sensor's medium (the fluid) and the skin is minimized. This reduces the reflections at the interface, allowing more light to enter the skin. The result is that the light returning to the detector or camera contains more useful information versus skin surface reflections.

In the context of optical sensing on skin, sensor movement relative to the tissue introduces a significant degree of variance in the signal received and the accuracy of the measurements obtained. This effect is markedly pronounced on surfaces with high reflectivity, such as skin, where the specular reflection of incident light can vary substantially with minor alterations in the relative position and orientation of the optical system components. These variations manifest as increased noise within the measurement data, as the optical sensor interprets these fluctuations in reflected light as meaningful changes in the observed parameters, thereby compromising the fidelity of the measurements.

The application of an index-matching fluid, designed to approximate the refractive index of skin, serves to attenuate the reflectivity at the skin surface. By harmonizing the optical impedance between the sensor and the skin, the index-matching fluid diminishes the specular reflection that otherwise exacerbates the noise introduced by sensor movement. Consequently, this reduction in surface reflectivity leads to a stabilization of the light dynamics at the interface, ensuring that minor positional adjustments of the sensor impart minimal disturbance to the path and intensity of the reflected light. This stabilization effect, therefore, substantially mitigates the influence of sensor movement resulting in measurement noise, thereby enhancing the precision and reproducibility of the optical measurements.

### Creating a Repeatable Cardiac State

The accurate assessment of aortic stenosis is facilitated by creating a repeatable cardiac state for the measurement period. The repeatable cardiac state is defined by conditions that create a consistent degree of myocardial muscle fiber stretch or tension before the start of ventricular contraction and a repeatable degree of sympathetic activation that affects cardiac contractility. A repeatable cardiac state is established when ventricular contraction is preload independent and under vagal control. The invention determines the presence of both conditions. Creating a repeatable cardiac state is desired for accurately assessing aortic stenosis as it reduces physiological noise and ensures consistent filling and contraction conditions for measurements. Preload independence and vagal control optimizes the heart's filling, allowing for maximal sensitivity to aortic stenosis. When the heart is filled at a maximal physiological level, the pressure gradient across the aortic valve increases, making it easier to detect any obstruction caused by the stenotic valve. This heightened pressure gradient provides clearer indications of the degree of narrowing in the aortic valve, which helps with diagnosing aortic stenosis. The resulting physiological standardization helps to improve accuracy and facilitates treatment decisions.

### Preload Independence

According to the Frank-Starling law, there is a positive relationship between preload and stroke volume; accordingly, the greater the ventricular preload (and therefore the degree of cardiac muscle stretch), the greater the stroke volume. However, this relationship, in the same way as in most physiological phenomena in the body, is not linear and traces a curve. Accordingly, once a level of preload value has been reached, further increments do not give rise to significant additional systolic volume elevations.

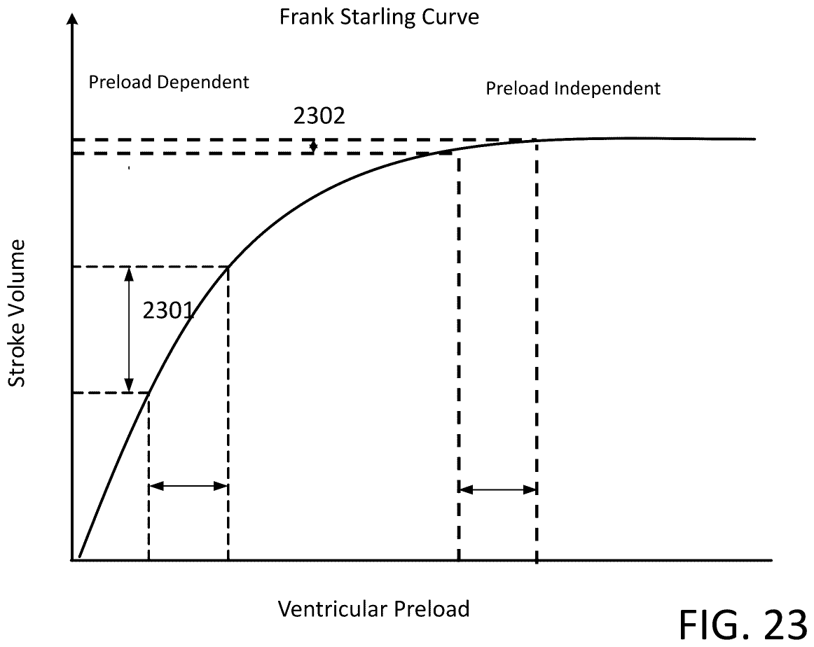

FIG. 23 illustrates the Frank-Starling curve with preload on the x-axis and stroke volume on the y-axis. The figure illustrates two zones of operation with dramatically different relationships between preload and stroke volume. The preload-dependent zone (2301) denotes a zone of operation where minimum preload changes give rise to a marked increase in systolic volume, which is known as preload dependence. The preload independent zone (2302) denotes a flatter zone of operations, where the ejection volume varies little with a change in preload, which is known as preload independence.

For aortic stenosis assessment, there are benefits to conducting the test during conditions of preload independence as it creates a repeatable amount of myocardial muscle fiber stretch or tension before the start of ventricular contraction. One method of achieving preload independence under typical physiological conditions is to have the patient in a supine position. A supine body position is typically associated with preload independence due to increased venous return. Movement from the standing position to the supine position results in the translocation of approximately 300 ml to 500 ml from the lower extremities towards the intrathoracic vessels and produces an increase in venous return and cardiac preload. However, the simple placement of the body in a supine condition does not guarantee preload independence. These conditions can be confirmed via additional assessments of interbeat time interval (IBI) and interbeat time interval variability.

### Accessing Preload Independence via Venous Return Change Evaluation

Preload independence can be accessed via activities that alter the venous return to the heart. The changes in venous return and corresponding preload should not result in a significant alteration in stroke volume if the patient's heart is operating on the plateau portion of the Frank-Starling curve, a condition of preload independence. There exist many mechanisms that can be used to alter venous return, including (1) intrathoracic pressure changes, (2) changes in the total circulating volume, and (3) alterations in the distribution or location of the volume. For aortic stenosis assessments, any dynamic alteration of venous return should be conducted so the patient remains unstressed, and time is allowed to obtain a physiological state of cardiac vagal control. For the determination of preload independence, the evaluation process involves a comparison of physiological parameters in the two venous return conditions. For example, suppose the condition of increased venous return increases stroke volume, as observed by an elongation of LVET or a decrease in heart rate. The comparison process can evaluate if the changes suggest a condition of preload dependence by comparing the physiological parameters to prior physiological measurements from the patient, to demographically matched references or defined thresholds. Deterministic outputs, as well as probabilistic assessments, can be generated. Multiple methods for changing venous return are described below and may be used independently or in combination to determine preload independence.

Intrathoracic pressure changes can be used to alter venous return to assess preload independence. During inspiration, venous return increases as the intrathoracic pressure becomes more negative. The reduction in intrathoracic pressure draws more blood into the right atrium. Multiple types of breathing protocols can be used to alter venous return with subsequent assessment of LVET to determine preload independence. Various methods for changing intrathoracic pressure and, correspondingly, venous return are explained subsequently.

The Valsalva maneuver is an exaggerated exhalation, usually a sustained, forced exhalation against a closed glottis. During a maintained increase in intrathoracic pressure, venous return is interrupted, and stroke volume falls.

Resistance breathing is a general term that applies to any method that increases, decreases, or changes intrathoracic pressure over normal breathing and alters venous return. A resistance breathing test can include inhalation resistance breathing or exhalation resistance breathing, independently or in combination. The use of exhalation resistance breathing creates an increase in intrathoracic pressure while the use of inhalation resistance breathing creates decreased intrathoracic pressure. CPAP is a form of resistance breathing, but where positive pressure is exerted over the entire breath cycle. Additionally, the system can use different levels of resistance over the course of the protocol. Multiple methods of implementation exist for altering intrathoracic pressure above normal levels. The system can include the use of pressure threshold, flow-independent valves, air restriction mechanisms, and any mechanism that causes an increase in pressure during normal breathing. Additionally, the term resistance breathing covers the process of creating a change in intrathoracic pressure where little or no air movement occurs for a period of time. The creation of an occlusion pressure, either increased or decreased, is encompassed as part of the broad definition of resistance breathing. Resistance breathing is a method that can be used to change venous return to the heart and influences end-diastolic volume.

Paced breathing is a general term that applies to any method that alters breathing rate by defining the rate and can include depth of breathing. Paced breathing is typically slow at a rate between 5 and 7 breaths per minute. With normal breathing, the rate is about 12 to 14 breaths per minute. Paced breathing can include defined changes in the rate as well as an asymmetric breathing profile, for example the exhale is 8 seconds while the inhale is 5 seconds.

Controlled breathing is the process of combining elements of paced breathing with resistance breathing. The “controlled” aspect is a system or method of breathing that dictates breathing rate and creates an intrathoracic pressure change that is greater than normal breathing. Examples of controlled breathing include but are not limited to a mini-Mueller inhale against resistance followed by a mini-Valsalva against resistance at a rate of 6 breaths per minute.

Changes in circulating volume by administration of IV fluids, often referred to as a volume challenge, can be used to alter venous return. Evaluation of the response to the administration of a given amount of volume (fluid challenge) can be used to access preload independence. Additionally, the execution of a hydration protocol can be initiated, and may include drinking fluids.

Body position changes are a simple and reliable method for altering the distribution of the circulating volume, changing preload, and evaluating preload dependence. For example, while supine, passively raising the legs to an angle of 45 degrees to the surface being laid upon for at least 1 minute is equivalent to a volume expansion of about 300 ml. The effect is only temporary so the maneuver is regarded as a test and can be repeated if necessary. The blood transfer from the lower extremities towards the intrathoracic vessels produces an increase in venous return and increased cardiac preload. Clinical studies have shown the usefulness of this maneuver in evaluating the response to volume expansion. These studies suggest that an increase of ≥10% in stroke volume during the first 60 to 90 seconds of the leg raising maneuver offers sensitivity and specificity performances of over 90% in predicting the capacity to increase stroke volume with the administration of fluids.

Before aortic stenosis assessment, a simple preload test can be conducted. The patient would start in the supine state with an LVET and interbeat interval obtained. The legs of the patient can be raised and the response due to blood moving out of the legs assessed by looking at the change in either or both LVET and interbeat interval. If the change were below a predetermined threshold, then the patent is in a preload independent state. If the change is larger than the defined threshold, the patient would be in a preload dependence state, and the criteria for making an aortic assessment were not satisfied.

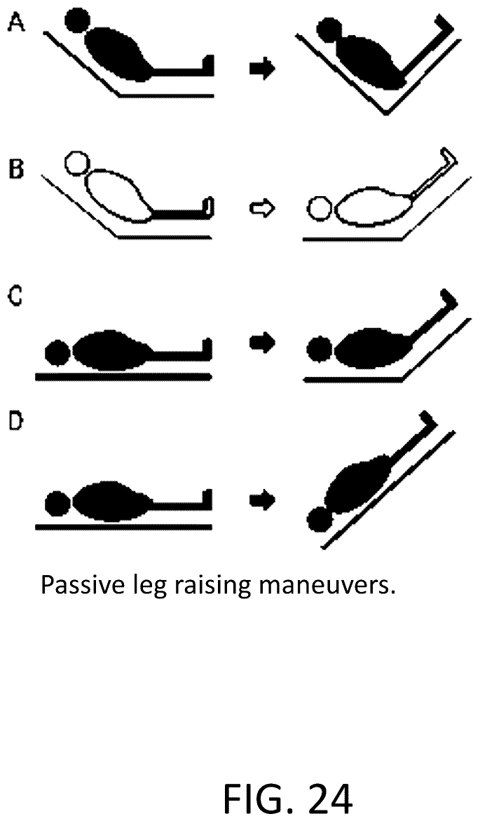

Other types of passive leg raising maneuver modalities can be used and are illustrated in FIG. 24. From the “semi-raised” position, the legs can be elevated without lowering the trunk. This maneuver involves a lesser risk of aspiration and elevation of intracranial pressure (ICP) but generates less volume expansion since the splanchnic blood volume is not included. From the “semi-raised” position, the legs can be elevated, and the trunk can be lowered to zero degrees. From the supine position, the legs can be raised 45 degrees without moving the trunk. The final maneuver involves rotation of the entire body. This maneuver causes significant changes in venous return but can result in some anxiety in the patient.

Any method or combination of methods that alters venous return can be used by the physiological assessment system for the assessment of preload independence. If the dynamic assessment of preload independence indicates preload dependence, the circulating volume of the patient can be altered by fluid consumption, or IV fluid administration and the patient retested.

For a reliable cardiac fitness assessment, the degree of change acceptable following a venous alteration will be defined and may be different based on the demographic and medical history of the patient, and the type of venous alteration used. In summary, preload independence can be accessed by examining one or multiple physiological parameters and determining if the percent change was less than a defined amount after the increase or reduction in preload. Candidate physiological parameters for preload assessment following venous changes include interbeat time interval and LVET.

### Inferring Preload Independence via Observational Parameters

During unstressed conditions, the body has a repeatable cardiac output requirement for the maintenance of metabolic functions. Cardiac output is the product of multiplying the stroke volume by heart rate (cardiac output=heart rate×stroke volume.) In the absence of changes in sympathetic tone, heart rate and stroke volume have an inverse relationship. The body has a defined cardiac output need, so if stroke volume decreases, then heart rate increases. Thus, heart rate can be used to access preload independence in an individual patient during the measurement period by conducting a comparative assessment and identifying a minimum or low heart rate. The low heart rate infers a high stroke volume which is linked to preload independence. The determination of a low heart rate for a given individual can occur via a comparative assessment based on historical heart rates from the user, other historical values, and other relevant comparison groups. Relevant comparison groups can include demographic matching, health status matching, medication matching, medical history match, or other relevant comparison groups.

For example, consider a measurement period where the patient sleeps in the supine position. The evaluation process for determining preload independence can include an assessment of body position and a comparative analysis of interbeat time intervals. The presence of a supine position can be determined in many ways, including direct measurements, inferred measurements or self-reported measurements. The effect of heart rate on ejection time interacts with body position. This physiological relationship was shown by Miyamoto et al, (Miyamoto, Y., Higuchi, J., Abe, Y., Hiura, T., Nakazono, Y., & Mikami, T. (1983). Dynamics of cardiac output and systolic time intervals in supine and upright exercise. Journal of Applied Physiology, 55(6), 1674-1681.).

After detection of a supine position, an interbeat time interval can be obtained, and a comparison assessment conducted. The comparison assessment can determine if the observed heart rate was a minimal heart rate or above average based on other measurements made during the assessment period, historical measurements from the patient, or relative to other matched patients. The comparative process can include demographic matching, health status matching, medication matching, medical history matching, or other relevant comparison groups. A higher heart rate can be associated with a lack of preload independence, so the criteria associated with the repeatable cardiac state are not satisfied and no cardiac assessment is initiated. If the observed interbeat time interval is consistent with preload independence, a qualification signal or validation signal is initiated, and a cardiac fitness assessment can be conducted or reported.

In practice, nightly sleeping represents an appropriate measurement period as the patient is in the supine position and the duration of the measurement is several hours. Thus, one or more minimal or low heart rate observations can be used independently or in combination to create a cardiac fitness score for a given measurement period.

The length of the assessment period needs to account for the realities of obtaining measurement data. If the measurement is made in the clinic, then the assessment period will be defined by the availability of the patient and medical providers. If the determination of aortic stenosis can be made in the home over a longer period, it may be desirable to account for hormonal fluctuations that occur over a month. For example, the menstrual cycle influences both heart rate and heart rate variability. Over the menstrual cycle, the female body undergoes many hormonal changes that affect resting heart rate, heart rate variability, and body temperature. On average, heart rate increases between two and three beats per minute during fertile days preceding the monthly period. With an objective of measuring repeatable cardiac state, the desired objective would be to avoid periods of hormonally elevated heart rate or have the assessment period span the variance in hormonal levels.

### Cardiac Vagal Control

For the purpose of aortic stenosis assessment, it is desired to have the contractility state of the muscle at a repeatable cardiac state with minimal influence of contractility or ionotropic agents. Specifically, autonomic sympathetic activation of the cardiac muscle should be minimal, as sympathetic activity impacts myocardial contractility far more than parasympathetic activation. General approaches to minimizing sympathetic activation include rest, calming environments, and sleeping. With respect to sleep, certain sleep cycles are considered more conducive to cardiac vagal control. Overall, sleep is considered a condition in which vagal activity is high and sympathetic activity is relatively quiescent.

Within sleep stages non-rapid eye movement (non-REM) and REM sleep have lower levels of sympathetic tone relative to the phasic bursts of rapid eye movements characteristic of REM sleep reflecting sympathetic activation.

### Accessing Cardiac Vagal Control via Physiological Measures

Cardiac vagal control is an autonomic state when the vagus nerve alters the interbeat time interval with high responsivity, precision, and sensitivity. Cardiac vagal control occurs when the parasympathetic nervous system exerts greater control over cardiac function (interbeat time interval and contractility) than the sympathetic nervous system, and sympathetic activation is low. Cardiac vagal control, as an autonomic state, can be inferred by using physiologically derived measures obtained noninvasively. Cardiac vagal control can be inferred using one or more of the following measures of respiratory sinus arrhythmia, interbeat time interval variability, vagal tone, the balance between the sympathetic and parasympathetic systems, a resting state, sleep state, low heart rate, and pulse variations.

Respiratory sinus arrhythmia is a physiological phenomenon where the heart rate accelerates during inspiration and slows down during expiration. Respiratory sinus arrhythmia (RSA) is frequently used as a noninvasive method for investigating vagal tone and is typically identified via electrocardiography (ECG) recording, PPG recording, SPG recording, and other noninvasive systems. Additionally, other methods have been developed that take advantage of the interactions between ventricular ejection and respiration. Interpretation of respiratory sinus arrhythmia measurements must be made with care, however, as several factors, including differences between individuals, can change the relationship between respiratory sinus arrhythmia and vagal tone. In practice, an estimate of respiratory sinus arrhythmia can be calculated by subtracting the shortest interbeat time interval during inspiration from the longest interbeat time interval during exhalation. RSA can be calculated via multiple methods, with some including breathing rate while others do not. The resulting RSA parameter is evaluated via a comparative assessment using historical values. The comparison assessment can determine if the observed RSA level is consistent with cardiac vagal control. The assessment of RSA can include other measurements made in an assessment period, historical measurements from the patient, or relative to other matched patients. The comparative process can include demographic matching, health status matching, medication matching, medical history matching, or other relevant comparison groups.

Heart rate variability (HRV) is the physiological phenomenon of variation in the time interval between heartbeats and is associated with respiratory sinus arrhythmia. It is measured by the variation in the beat-to-beat interval and has been described by more than 70 variables. HRV analysis can be performed in the time domain, in the frequency domain, and with non-linear indices.

Vagal tone can be accessed by examination of the high-frequency components of HRV. High-frequency heart rate variability is a frequency domain analysis typically between 0.15 and 0.40 Hz and is commonly associated with vagal tone.

The balance between the sympathetic and parasympathetic systems can be assessed by examination of the low frequencies/high-frequencies ratio, where low frequencies are typically defined as between 0.04 and 0.15 Hz.

A resting state is defined by the lack of significant volitional activities by the patient. Accelerometers in the measurement device are commonly used to assess the movement of the patient.

Low heart rate is evaluated using a naïve reference based on demographically matched values or prior observations with the patient.

The presence of sleep and the identification of the sleep stage can be based on heart rate variability. During normal sleep, the autonomic nervous system (ANS) modulates cardiovascular functions during sleep onset and the transition to different sleep stages. The analysis of heart rate variability (HRV) is a reliable tool to assess cardiovascular autonomic control as it can report physiological autonomic changes present during the wake-to-sleep transition, sleep onset, and different sleep stages: REM and NREM sleep. In addition to heart rate variability, heart rate, breathing rate, skin temperate, movement information and the time of day can be used to determine the presence of sleep. These parameters are used by the sleep assessment system to determine the presence of sleep as well as a sleep stage. The presence of sleep and a non-rapid eye movement sleep stage are associated with cardiac vagal control and can be used for the determination of cardiac vagal control.

The determination of cardiac vagal control can involve one or more of the above assessments, as well as additional metrics. These one or more metrics are evaluated by comparison to prior physiological measurements from the patient, the identification of physiological extrema, and the comparison to demographically matched references or defined thresholds. Deterministic outputs, as well as probabilistic assessments, can be generated.

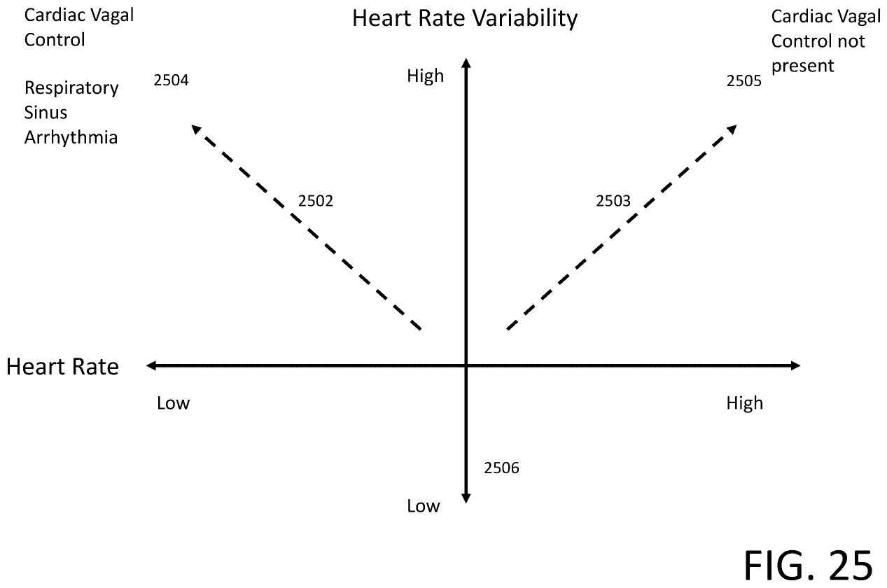

The presence of cardiac vagal control can be illustrated effectively in an XY coordinate system. FIG. 25 is an illustration demonstrating the relationship between several key measurement parameters and illustrates that heart rate variability cannot be used exclusively for the determination of cardiac vagal control. The X-axis is heart rate, and the Y-axis being heart rate variability. For left ventricular outflow tract assessment, the desired location is in the upper left, with low heart rate, high heart rate variability, and the presence of respiratory sinus arrhythmia. The degree of respiratory sinus arrhythmia is illustrated by line (2502). A cardiac physiological condition satisfying the presence of respiratory sinus arrhythmia, high heart rate variability, and low heart rate is illustrated by numerical reference (2504) and would be consistent with cardiac vagal control. Numerical location (2505) illustrates a cardiac physiological state undesired for aortic stenosis assessment because the physiological source of heart rate variability is not concurrently observed with a low heart rate. Locations with low heart rate variability, unless due to a disease condition or medications, as shown by numerical reference (2506) are not a desired physiological state for aortic stenosis assessment and can be due to multiple etiologies, including sympathetic tone, exercise, catecholamines, or medications. The use of heart rate and heart rate variability creates a coordinate system for the determination of cardiac vagal control. During testing, the physiological assessment system determines if desired preload conditions and autonomic nervous system conditions are present by determining the presence of preload independence and cardiac vagal control. In the presence of preload independence and cardiac vagal control, a pulse plethysmogram is acquired over at least on cardiac cycle. The singular or combined use of preload independence and cardiac vagal control define two easily measured parameters that can be used to create a repeatable cardiac state.

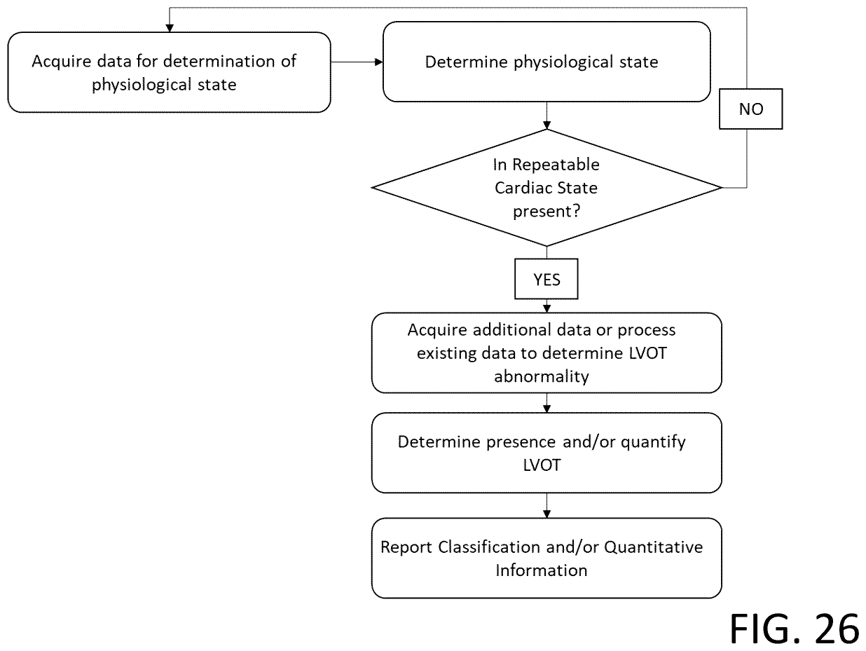

FIG. 26 illustrates a flow chart associated with use of the invention. The system can acquire pulse plethysmogram information and then use the data to determine the presence of the desired physiological state. If the desired physiological state, such as a repeatable cardiac state, (as illustrated) is present, then the data used to determine the physiological state or additional data are processed to determine the presence of a left ventricular outflow tract abnormality and to provide quantitative information as desired.

For the measurement of left ventricular outflow tract (LVOT) abnormalities, including aortic stenosis, the inclusion of a qualification or validation step is proposed as a method to enhance the accuracy and repeatability of the results. This step could include verifying that the patient is in a physiological state that is conducive to precise and reliable measurements. The critical physiological state assessments for this qualification include: (1) preload independence, to ensure a repetable degree of cardic stretch; (2) cardiac vagal control, to ensure a repetable degree of contractility; and (3) a repeatable cardiac state, to ensure the conditions under which measurements are taken can be consistently replicated. This qualification or validation information can either accompany the LVOT measurement results as supplementary data or be employed as a prerequisite condition before processing the data of LVOT assessment or providing a measurement resuls. The flexibility in implementation allows for the adaptation of this process to various clinical settings, but the overarching aim remains to qualify measurements in a manner that promotes the generation of more accurate and repeatable results. This approach underscores the importance of establishing a stable physiological baseline, from which meaningful assessments of LVOT abnormalities, such as aortic stenosis, can be accurately determined.

### Monitoring Disease Progression

Monitoring the progression of aortic stenosis (AS) is critical for timely and effective patient management. The progression rate of AS varies significantly among individuals, influencing the optimal timing for surgical or interventional treatment. A systematic review by Heuvelman et al. (2012) emphasizes the importance of accurate estimates of AS progression for appropriate surveillance and treatment planning. (Heuvelman, Helena J., et al. “Progression of aortic valve stenosis in adults: a systematic review.” J Heart Valve Dis 21.4 (2012): 454-62.) This review, which included 27 reports with a total of 4,921 patients, highlighted the variability in AS progression rates and the methodologies used to assess this condition. Specifically, the review found pooled annual progression rates based on hemodynamic variables, suggesting a need for standardized assessment methods, such as measuring the aortic valve area (AVA), to better understand AS progression determinants and tailor treatments effectively. The study calls for the adoption of a universal method for AS assessment to facilitate insights into AS progression and enable evidence-based treatment customization. Monitoring AS progression, particularly changes in AVA, is thus essential for optimizing patient care and outcomes.

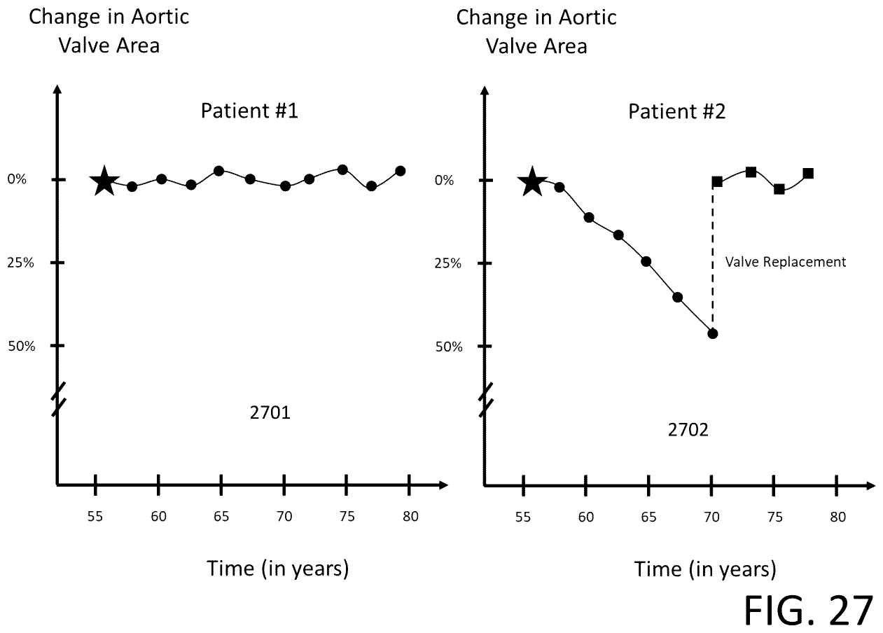

Quantitative measurements made with the patient in a repeatable cardiac state allow relative change or self-reference measurement with improved precession. Self-reference measurements involve comparing a current measurement to a previous measurement from the same patient using a similar measurement process. Self-referenced measurements in monitoring aortic stenosis (AS) progression reduce measurement error by removing common biases in the measurement. This approach eliminates one degree of freedom from the measurement, enhancing accuracy. By comparing an individual's data over time, specific measurement inconsistencies that might affect cross-sectional comparisons are minimized. FIG. 27 is an illustration of such a process.

Utilizing quantitative discrete change measurements in AS allows for the detection of subtle disease progression nuances, potentially obscured by generic criteria. Personalized tracking ensures interventions and treatment plans are more accurately aligned with the individual's disease trajectory. This method focuses on changes relative to the patient's baseline, or prior measurements, providing a clearer picture of progression removed from the generalizations inherent in population-based data and population-based management protocols.

### Left Ventricular Outflow Tract Assessment System

The left ventricular outflow tract assessment system can perform one or more of three related functions: the determination of the presence or absence of a left ventricular outflow tract abnormality of a flow anomaly, the classification of the anomaly, and provision of a metric or quantitative result regarding the severity of outflow tract obstruction. Anomaly detection is the identification of optical pulse plethysmographs that deviate significantly from the majority of the data or normal physiology. The result is a two-class determination, normal versus abnormal. The process can be extended to multiple classes:

normal, valvular obstruction, supravalvular obstruction, or subvalvular obstruction. Additional classifications can be added if desired clinically and supported by the measurement system. Finally, the assessment system can produce a continuous assessment variable associated with the degree of obstruction. For example, the assessment system can provide an assessment of the aortic valve area which corelates with the severity of stenosis.

The left ventricular outflow tract assessment system utilizes sophisticated analysis methods for the detection of a flow anomaly and the classification of the anomaly. The assessment system performs mathematical data processing steps without patient/user involvement. The analysis method can include many classes of models but can be broadly broken into “prediction models” and “matching models.”

Prediction models are constructed by determining the relationship between data or data features and desired output. The determination of relevant data features can be facilitated by morphological waveform analysis. Morphological waveform analysis is a technique used to analyze and understand the behavior of a time-varying signal by decomposing the measured signal into its constituent waveform shapes or “morphs.” This can be useful for identifying patterns or trends in the signal, as well as for identifying and isolating specific features or events within the signal that are associated with the measurement output of the system. The process of morphological waveform analysis typically involves three main steps:

Decomposition: The signal is decomposed into its constituent waveform shapes, or morphs, using a morphological filter. This filter is designed to identify and extract the underlying waveform shapes within the signal and can be applied either in the time domain or the frequency domain.

Classification: The extracted morphs are then classified based on their shape, size, and other characteristics, such as their duration, amplitude, or frequency content.

Analysis: The classified morphs are then analyzed to understand their behavior and their role in the overall signal. This may involve comparing morphs to each other, examining their statistical properties, or using them to identify trends or patterns within the signal.

Morphological waveform analysis is particularly useful for analyzing signals that are complex or noisy, as it allows analysts to extract and analyze the underlying waveform shapes that contribute to the overall signal.

Upon identification of data features, a prediction model can be developed. A prediction model is a mathematical model that uses a set of data features to generate a prediction result. These data features, also known as variables or predictors, can include any measurable attributes or characteristics of the data being analyzed. The model uses a set of algorithms to analyze the relationships between the data features and the prediction result, and then generates a prediction based on that analysis. Examples of prediction models include regression models, where features are mapped to outputs through linear or non-linear relationships, as well as some machine learning models.

The goal of the prediction model is to accurately predict the outcome of a particular event, presence or absence of a left ventricular outflow tract abnormality or the type or severity of the abnormality. In one potential application, the prediction model may predict the valve area in aortic stenosis. Once the model relationship is determined, the model can be applied to novel data without relying on training or reference data. The development of training of the prediction model can use a variety of training techniques including supervised detection techniques, semi-supervised detection techniques, and unsupervised detection techniques.

Matching models are used to identify and match patterns or relationships between different pulse waveforms and the clinical condition, effectively creating a function map between the input and output variables. These models can be developed using various methods, including traditional machine learning techniques such as linear regression or decision trees. However, more recently, deep learning techniques have also been applied to the development of matching models. Deep learning involves the use of artificial neural networks, which are designed to mimic the way the human brain processes information.

These neural networks can be trained on representative data and can learn to recognize patterns and relationships, making them particularly well-suited for matching tasks. Some common deep learning techniques used in the development of matching models include convolutional neural networks, recurrent neural networks, and autoencoders.

Recurrent neural networks (RNNs) are a type of artificial neural network that are particularly well-suited for processing sequential data. They are called “recurrent” because they have a feedback loop in their architecture, allowing them to retain information from previous time steps and use it to inform their processing of the current time step. This capability makes them effective at tasks involving time series. One area where RNNs have been applied is in the analysis of physiological pulse data. For example, RNNs have been used to predict heart rate and respiratory rate from pulse waveforms. In these cases, the RNN can learn patterns in the pulse data over time, allowing it to make more accurate predictions. RNNs have also been used to classify pulse waveform patterns, such as identifying the presence of arrhythmias or identifying different types of arrhythmias. Overall, RNNs offer a powerful tool for understanding and interpreting physiological pulse data and are applicable to pulse plethysmograph data.

In these approaches, listed above and often referred to as “deep learning models,” the useful features and representations are essentially learned by the model in training, along with the function that maps the inputs to the desired outputs. The models are developed using the measured signals from the sensor system, in raw or conditioned data representations that contain the left ventricular outflow tract abnormality and an output variable indicative of the degree or presence of the left ventricular outflow tract abnormality. Because the relationship between input and outputs is often quite complex (involving thousands of weights in multiple hierarchical layers), the engineer or architect of the model might be completely unaware of the features or information that the model has extracted or how and why that information is combined to form the output.

Models using hierarchical layers can divide the data into levels, with each level representing a different level of aggregation or a different aspect of the data. Hierarchical models allow for the modeling of both within-level and between-level variation and can account for the dependencies between observations at different levels. Hierarchical models can be more complex than other types of models and can require more computational resources to estimate, but they can be useful for handling complex data and making nuanced predictions.

For both prediction and matching models, the desired output can be a continuous variable (e.g., a degree of flow abnormality) or a binary variable that takes on values of zero/one, indicating the presence or absence of a specific condition or state (e.g., the presence of a left ventricular outflow tract abnormality). Predictive algorithms use mapping functions to define a mathematical function between inputs to outputs, where the inputs are the historical data, and the outputs are the predictions.

The mapping function of a predictive algorithm is typically defined by a set of parameters that are learned from the historical data during the training process. The set of parameters defines a mathematical mapping function between the input information (pulse plethysmogram) and information on the presence or absence, type of obstruction, or severity of a left ventricular outflow tract obstruction. Different types of predictive algorithms have different types of parameters and different ways of learning them. For example, linear regression models have coefficients that are learned by minimizing the sum of squared errors between the predictions and the true values. Decision trees have a tree structure that is learned by recursively splitting the data into subsets based on the values of the input variables. Neural networks have weights and biases that are learned by adjusting the internal parameters of the network using an optimization algorithm such as gradient descent. The output of the mapping function can be a binary output, a multi-class classification output or a continuous variable output. In the case of a multi-class classification the classification can be by the type of outflow tract obstruction.

Once the parameters of the predictive algorithm are learned, the mapping function can be used to make predictions on new input data by applying the learned parameters to the input data. The predictions made by the algorithm are estimates of the future events or outcomes based on the historical data.

The left ventricular outflow tract assessment system can use one or more mapping functions to define a relationship between inputs (historical data) to outputs (predictions), with the function being defined by a set of parameters that are learned from the historical data during the training process. Different types of predictive algorithms have different types of parameters and different ways of learning them, but all have a mapping function that can be used to make predictions on new input data.

The implementation of the left ventricular outflow tract assessment system for use on a medical device can use a number of hardware and software elements, often present in a programable data processing system, including:

Processor: The left ventricular outflow tract assessment system uses a moderately powerful processor, such as a central processing unit (CPU) or a graphics processing unit (GPU), to handle the computations required by the algorithm during the prediction phase.

Memory: The left ventricular outflow tract assessment system uses sufficient memory, such as random-access memory (RAM), to store the data and intermediate results during the execution of the algorithm.

Storage: The left ventricular outflow tract assessment system uses storage space to store the mapping function such as a deep learning algorithm and any additional data required by the algorithm during execution.

Connectivity: If the left ventricular outflow tract assessment system needs to communicate with other devices or servers to obtain or transmit data, it needs appropriate connectivity options, such as Wi-Fi or cellular connectivity.

Operating System: The left ventricular outflow tract assessment system can have an operating system that is compatible with the deep learning framework and libraries being used.

Frameworks and Libraries: The left ventricular outflow tract assessment system can have the appropriate algorithm frameworks and libraries installed in order to execute the algorithm. Examples of popular deep learning frameworks are TensorFlow, PyTorch, Caffe, etc.

Power: The left ventricular outflow tract assessment system can have enough power supply to run the deep learning algorithm, as running deep learning algorithms can require high computational power and can drain the battery quickly.

Data pre-processing: The left ventricular outflow tract assessment system can have software that pre-processes the data before it is analyzed by the deep learning algorithm.

The requirements can vary depending on the specific algorithm and device. For example, a device that is designed to use a prediction algorithm can have different requirements than a device that is using a deep learning algorithm.

The left ventricular outflow tract assessment system includes the totality of activities that process the measured data and data inputs and applies calculations or a designated set of steps to determine the presence of a flow abnormality and, to the extent possible, the structural or valvular abnormalities causing the obstruction, and a quantitative assessment. The left ventricular outflow tract assessment system can use a variety of inputs to include flow waveforms, volume waveforms, both volume and flow waveforms, measured signals obtained during volitional maneuvers, the inclusion of ancillary information and ancillary signals.

### Example Embodiments

The ability to conveniently detect structural and valvular abnormalities of the left ventricular outflow tract remains a desirable but currently unfulfilled objective. The invention addresses historical limitations in noninvasive optical determination of structural and valvular abnormalities in the left ventricular outflow tract by (1) utilizing improved optical measurement systems, (2) sampling locations that maximize the information content of the pulse waveform, (3) conducting volitional patient maneuvers to improve the diagnostic ability of the system, and (4) utilizing pulse enhancement techniques, (5) a physiological assessment system configured to analyze the measured systolic time interval information to determine the presence of a repeatable cardiac state; (6) a qualification or validation system configured to indicate the presence of a repeatable cardiac state, and (7) a left ventricular outflow tract assessment system configured to analyze the optical pulse plethysmogram to determine a quantitative assessment of aortic stenosis. The following example embodiments specifically address these historical limitations such that effective screening for left ventricular outflow tract abnormalities can be achieved.

Measurement Configuration. Depending upon the diagnostic situation, the measurement system can be configured to provide the degree of measurement resolution needed. The measurement configuration used can be determined on a per patient basis, at the clinic level or at the health system level and is based in part on the desired performance of the system, the cost of the instrumentation, and the sophistication of the operator. For example, the system can be configured to provide a simple screen test for any type of left ventricular outflow tract abnormality. The objective of the measurement can be to detect any abnormality so subsequent echocardiogram follow-up is initiated. In such a scenario, the objective is to have high sensitivity for left ventricular outflow tract abnormalities and reasonable specificity.

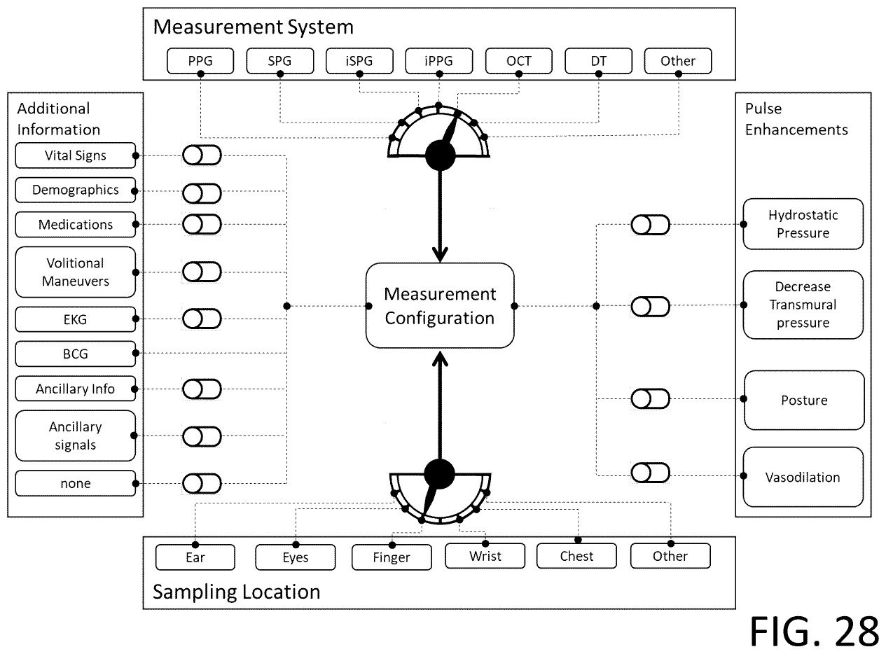

In another example, the objective can be to determine the location of the left ventricular outflow tract and to provide some staging information regarding severity. These two measurement applications would result in different measurement configurations. FIG. 28 is an illustration to show the measurement configuration concept. At the top of the diagram is the measurement system to be selected for procuring the measurement signal. At the bottom of the graph is the sampling location. The schematic illustrates that one type of measurement device is used, and one location is sampled. Multiple measurement systems and locations can be used, but for communication purposes and simplicity of illustration, only single selections will be discussed. Additional or ancillary information and ancillary signals are pictorially illustrated on the left side of the illustration. Correspondingly, the pulse enhancement techniques can be selected if needed from the right side of the illustration.

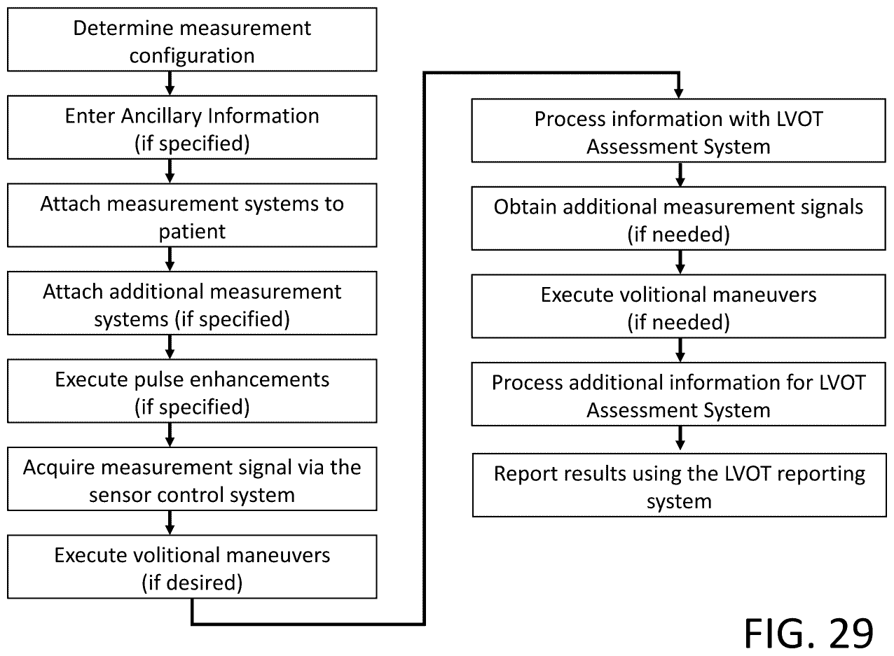

FIG. 29 shows an example of one proposed flow chart of the measurement process. As illustrated the measurement configuration defines the operation of the system. The procurement of the measurement signal is managed by sensor control system, and the resulting data stream is processed by the left ventricular outflow tract assessment system. Additionally, the flow chart illustrates the possible inclusion of additional measurements and the use of volitional parameters as both an element of the configuration and included in every measurement or as additional elements if the measurement result has uncertainty and additional diagnostic information is desired.

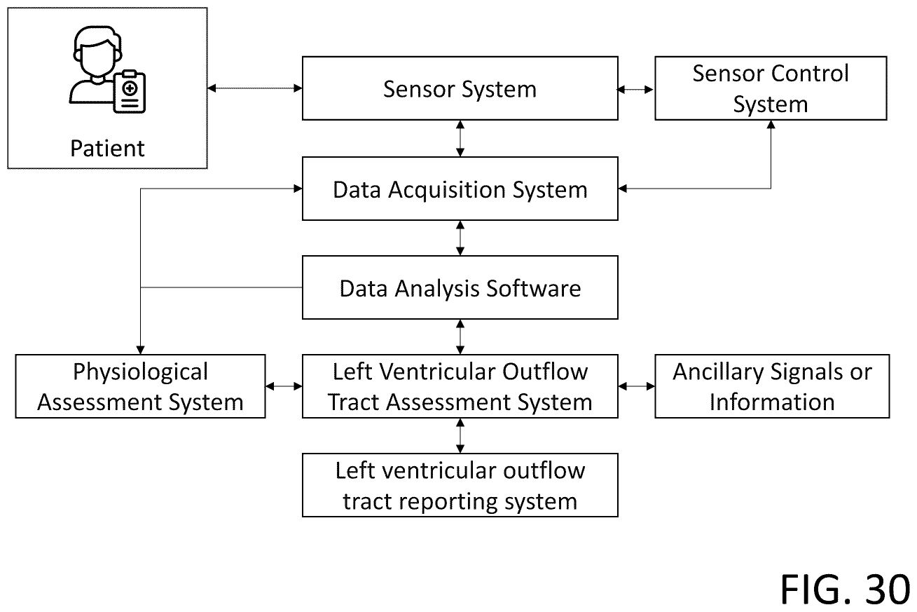

FIG. 30 is a general system diagram illustrating the major system elements. The sensor system is attached to the patient (details not shown). The sensor system is connected to the sensor control system. The sensor control system controls the sensor. The data acquisition system receives measurement data from the sensor system and may receive sensor control information from the sensor control system. The resulting measurement data is processed by the data analysis software and used by the left ventricular outflow tract assessment system. The physiological assessment system can be connected to the data acquisition system for access to measurement data or to the data analysis software for processed data. The left ventricular outflow tract assessment system may receive qualifying or validation information from the physiological assessment system and ancillary signals or information. The left ventricular outflow tract assessment system can process the received information to determine the presence or absence of a left ventricular outflow tract abnormality and the type of abnormality present, as well as provide a quantitative assessment of the degree of abnormality.

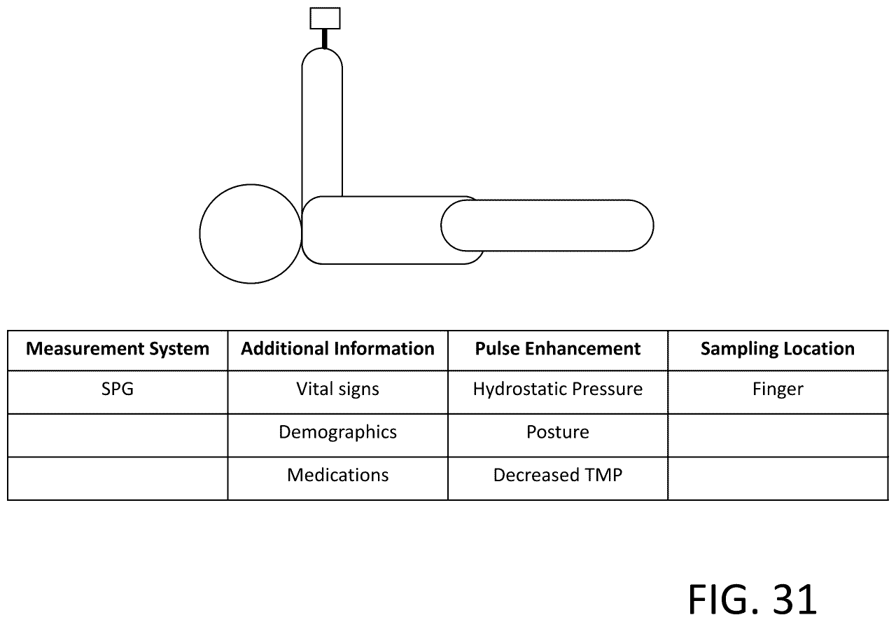

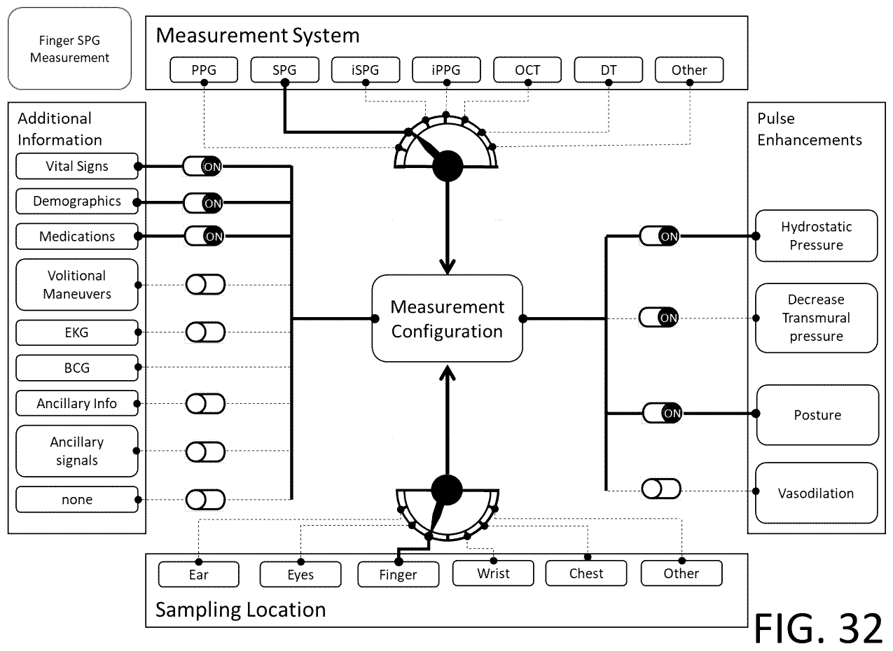

Finger SPG measurement embodiment. FIG. 31 illustrates a simple pictorial diagram of the measurement configuration and the key elements of a Finger SPG measurement embodiment. FIG. 32 illustrates the measurement configuration used for the illustrated embodiment. The system uses speckle plethysmography as the measurement method to acquire an optical pulse plethysmogram with diminished sensitivity to vascular tone changes in the hand and the impact of vascular stiffness changes in the conduit arteries. Additional information including vital signs, demographics, and medications are included and used by the analysis system. The pulse at the measurement site is enhanced by raising the arm above the heart, by having the patient in a supine position such that maximal filling of the heart occurs, and by having the measurement system exert additional external pressure at the measurement site, so transmural pressure is further reduced. The sampling location is the finger, a readily accessible location requiring little operator expertise. The resulting SPG measurement signal will be processed by the left ventricular outflow tract assessment system to determine abnormalities.

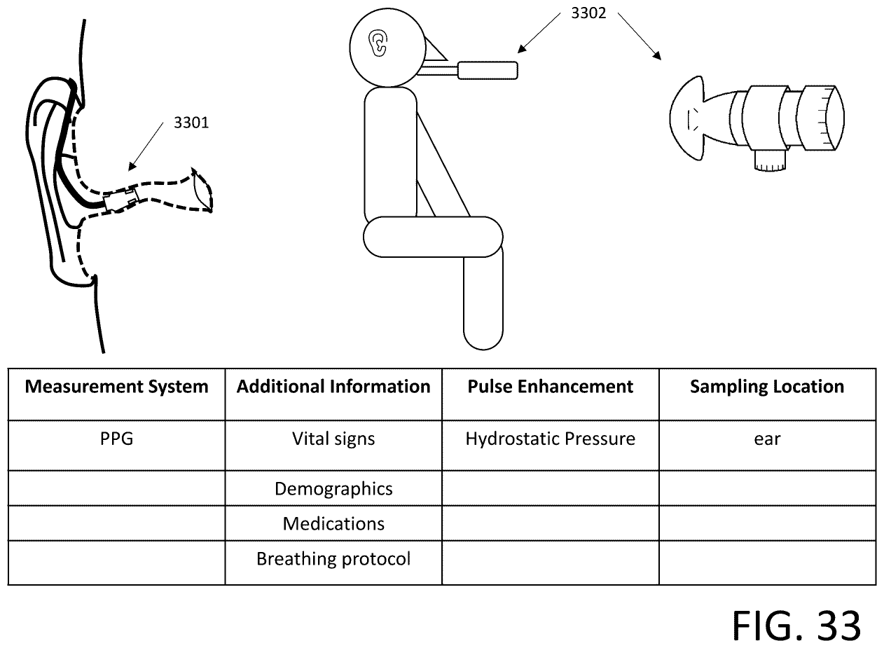

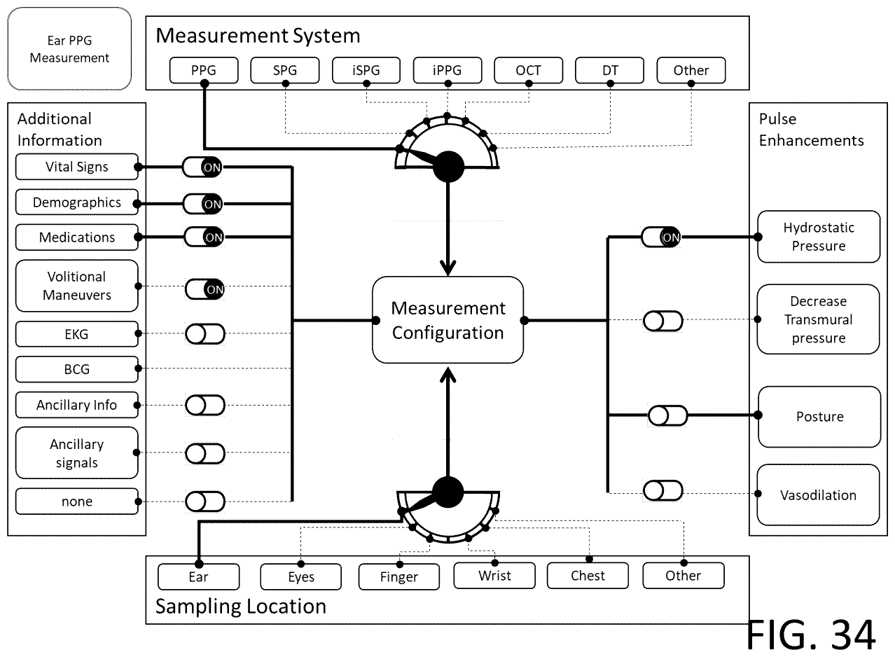

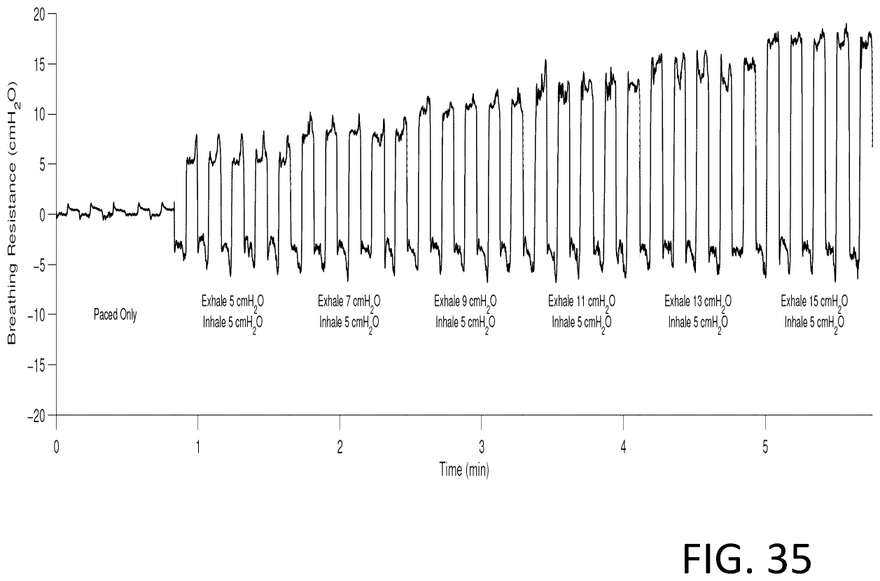

Ear PPG measurement embodiment. FIG. 33 illustrates a simple pictorial diagram of the measurement configuration and the key elements of an ear PPG measurement embodiment. FIG. 34 illustrates the measurement configuration used for the illustrated embodiment. The system uses photoplethysmography as the measurement method in the ear canal (3301) to acquire an optical pulse plethysmogram. The ear represents a sampling location with minimal vascular tome changes. Additional information can include vital signs, demographics, and medications are included and used by the analysis system. The measurement process, as illustrated, shows a volitional activity requiring the patient to execute a breathing protocol with a resistance breathing device, 3302. FIG. 35 illustrates such a protocol where the patient executes a controlled breathing protocol with constant inhalation pressure and step increases in exhalation pressure. The breathing device uses a flow-independent pressure threshold valve on both inhalation and exhalation to create the square wave pattern shown. A flow-independent pressure threshold valve is designed to provide consistent and specific pressure, regardless of how quickly or slowly the patient breathes. Threshold devices incorporate flow-independent one-way valves to provide consistent resistance and feature adjustable specific pressure settings. The breathing device creates a constant intrathoracic pressure during these segments. The resulting protocol or variances will alter the filling pressure to the heart in a deterministic fashion creating additional information for assessing left ventricular outflow tract abnormalities. The pulse at the ear measurement site is enhanced by hydrostatic pressure decreasing the transmural pressure, will experience minimal changes in vascular tone, as is a sampling location rich with reflection information. The sampling location is the ear, readily accessible and requires little operator expertise.

The resulting PPG measured signal obtained during execution of the measurement protocol can be processed by the left ventricular outflow tract assessment system in multiple ways. Specifically features can be extracted from the data stream or the totality of information used for analysis as a continuous data stream, or a combination of both. The left ventricular outflow tract assessment system then provides a measurement result which can be a probability score associated with left ventricular outflow tract abnormality or a specific diagnosis and a severity score.

The above configuration is also suitable, with modification, for the sampling of the carotid artery. The carotid arteries originate posterior to the sternoclavicular joints and in the neck and are contained within the carotid sheath posterior to the sternocleidomastoid muscle. The arteries represent a sampling location that can be accessed by SPG or PPG using infrared light.

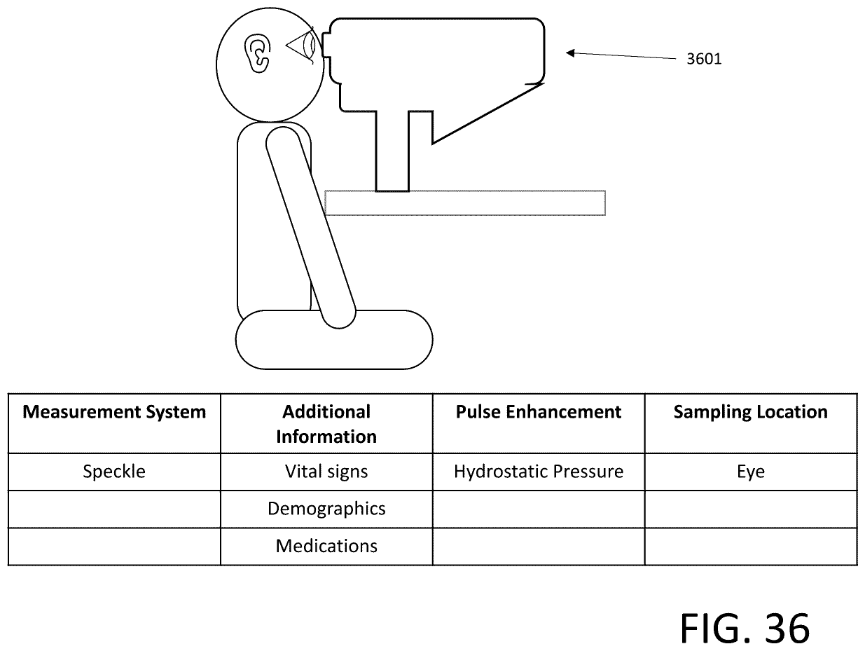

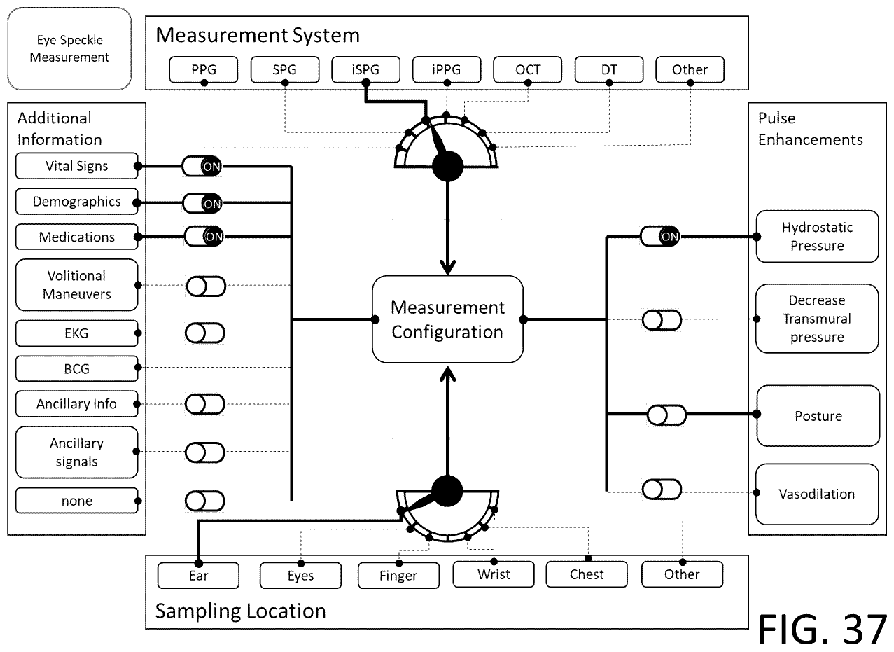

Eye laser contrast speckle imaging embodiment. FIG. 36 illustrates a simple pictorial diagram of the measurement configuration and the key elements of an eye laser contrast speckle imaging embodiment. FIG. 37 illustrates the measurement configuration used for the illustrated embodiment. The system uses speckle imaging or iSPG (3601) in optical communication with the retinal as the measurement method to obtain an optical pulse plethysmogram based on blood flow in the fungus, which includes the retina, macula, fovea, and posterior pole. The eye represents a location of stable vascular tone and is close to the heart. Additionally, the eye creates a unique optical access method such that larger arterial vessels can be sampled directly with a minimal scatter of the photons. Additional information can include vital signs, demographics, and medications and is used by the analysis system. The measurement requires alignment of the optical system so the retina can be visualized as the patient remains still. The pulse at the eye measurement site is enhanced by hydrostatic pressure decreasing the transmural pressure. The sampling location is the eye which may require some specific operator training.

Speckle imaging allows for determining a pulse plethysmogram for a region of interest. The region of interest can be a large area of the retina, freehand region of Interest (ROI), and cross-section of an artery or even a vein. The resulting SPG measurement signal obtained during the execution of the measurement protocol can be processed by the left ventricular outflow tract assessment system in multiple ways. Specifically, features can be extracted from the data stream, or the totality of information used for analysis as a continuous data stream, or a combination of both. Additionally, the entire video image or a region of interest can be processed by the left ventricular outflow tract assessment system. The left ventricular outflow tract assessment system then provides measurement results which can be simply a probability score associated with a left ventricular outflow tract abnormality or a specific diagnosis and a severity score.

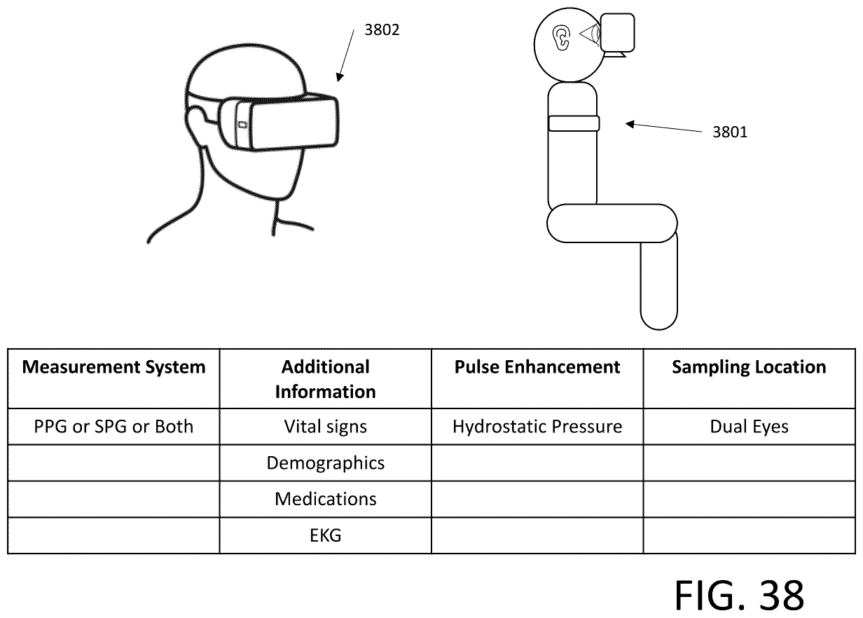

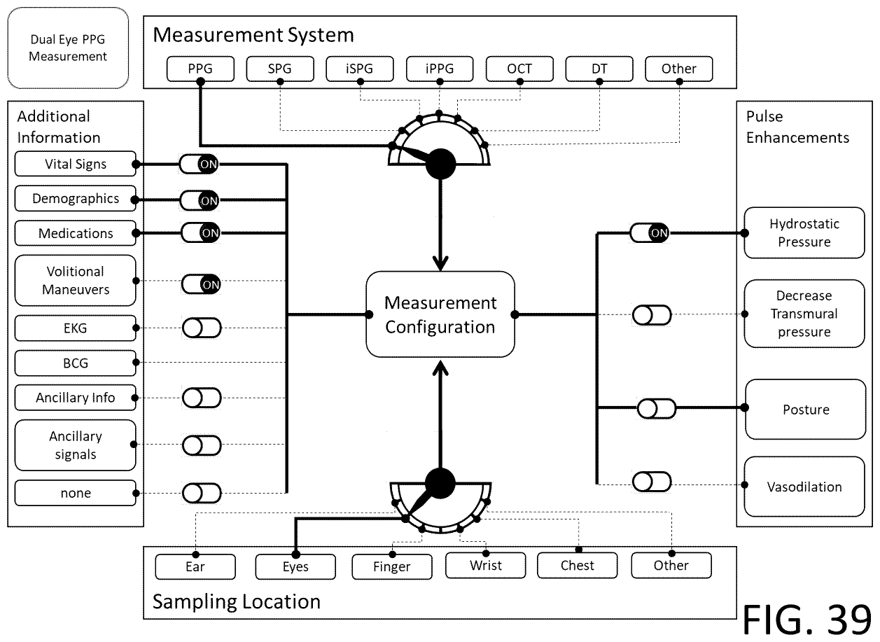

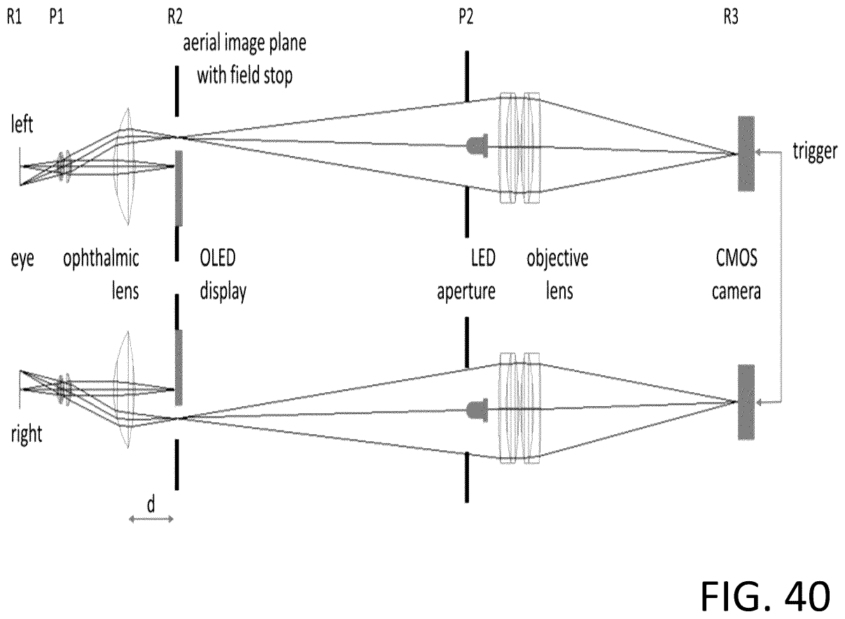

Dual Eye PPG imaging with ECG embodiment. FIG. 38 illustrates a simple pictorial diagram of the measurement configuration and the key elements of a dual eye PPG imaging with ECG embodiment. FIG. 39 illustrates the measurement configuration used for the illustrated embodiment. FIG. 40 shows an example optical layout for creating optical communication with both eyes enabling a dual PPG or SPG sampling of the eyes. The system, as illustrated, uses photoplethysmography as the measurement method in the eye. The eye represents a sampling location with minimal vascular tone changes. Additional information such as vital signs, demographics, and medications are included and used by the assessment system. An EKG measurement, 3801 is also associated with the measurement to facilitate the calculation of pulse arrival time (PAT). The presence of aortic stenosis can cause variances in PAT between the right and left sides of the body. The pulse at the eye measurement site is enhanced by hydrostatic pressure decreasing the transmural pressure. The sampling system is located in a single headset, 3802, that creates a dark environment for pupil dilation and provides instructional information to the patient.

The PPG signals obtained from both eyes and the EKG information can be processed by the left ventricular outflow tract assessment system in multiple ways. Specifically, features can be extracted from the data stream, or the totality of video information used for analysis as a continuous data stream, or a combination of both. The left ventricular outflow tract assessment system then provides measurement results which can be a probability score associated with a left ventricular outflow tract abnormality or a specific diagnosis and a severity score.

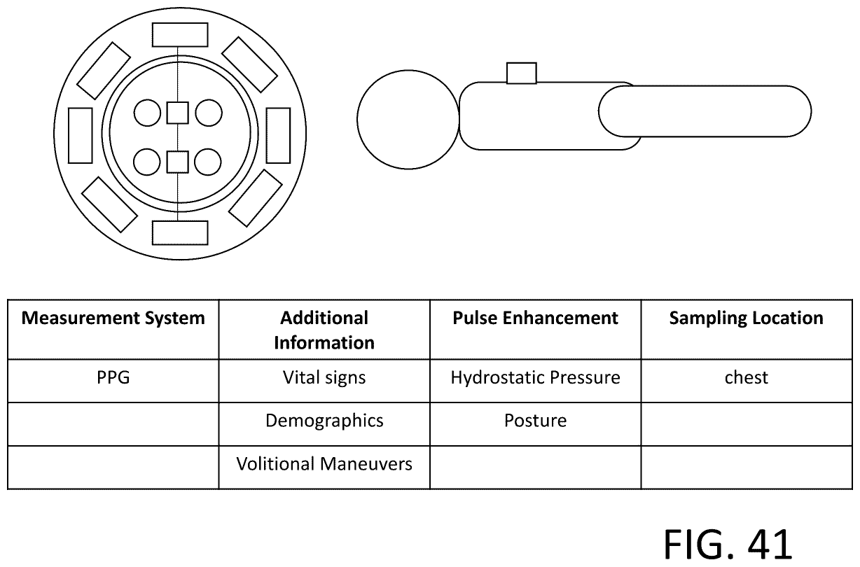

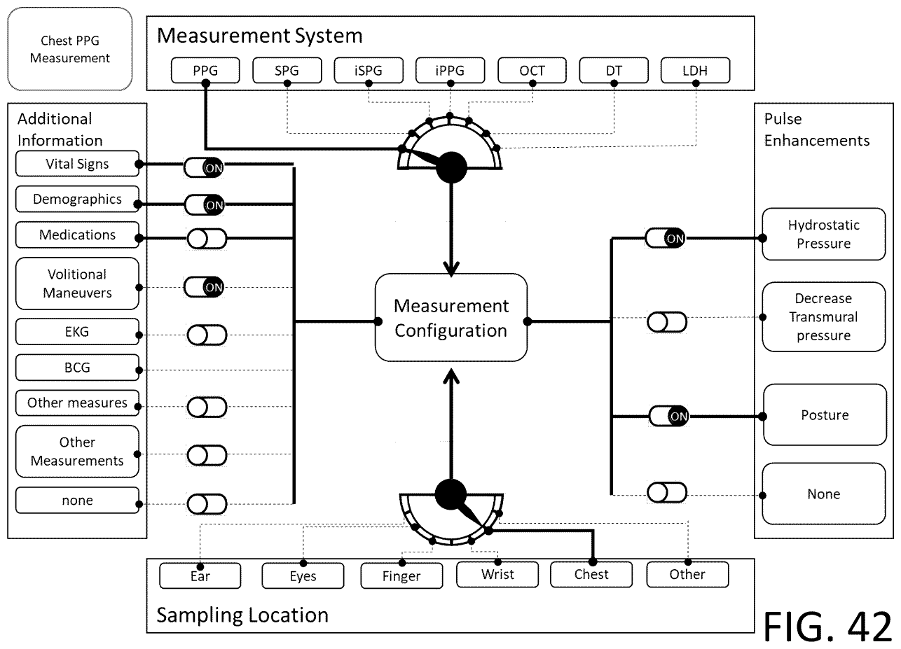

Chest PPG or SPG. FIG. 41 illustrates a simple pictorial diagram of the measurement configuration and the key elements of a chest PPG or SPG embodiment. FIG. 42 illustrates the measurement configuration used for the illustrated embodiment to include volitional maneuvers. Specifically, the measurement protocol implements a venous return change evaluation by acquiring optical sensor data while the patient is in the supine position. Following data procurement, a change in venous return is implemented by having the patient cooperate in a passive leg raise. Additional optical sensor data is obtained under the altered venous return condition. The change in cardiac function is evaluated to determine if preload independence is present. If preload is present, the physiological state of preload independence is qualified or validated. FIG. 26 is a flow chart depicting a process for determining a given physiological state. The left ventricular outflow tract assessment system uses the resulting physiological state information as illustrated in the system diagram of FIG. 30.

The system uses photoplethysmography as the measurement method on the chest to obtain an optical pulse plethysmogram. The chest represents an optical sampling location with very little transit distance in the arteries from the heart. Additionally, the system can use an optical system of significant size and power consumption. Such a measurement system can use a multi-detector, multi-source, and multi-wavelength configuration for the procurement of the best pulse waveform. Additional information can be included a phonocardiogram, not shown measure the intensity and location of the heart murmurs. The pulse is enhanced via a small hydrostatic pressure influence, the possible weight of the measurement system, and the patient's position in a supine position.

The resulting PPG or SPG signals obtained from the chest will be processed by the left ventricular outflow tract assessment system in multiple ways. Specifically, features can be extracted from the data stream, or the totality of video information used for analysis as a continuous data stream, or a combination of both. The left ventricular outflow tract assessment system then provides a measurement result which can be a simple probability score associated with a left ventricular outflow tract abnormality or a specific diagnosis and a severity score.

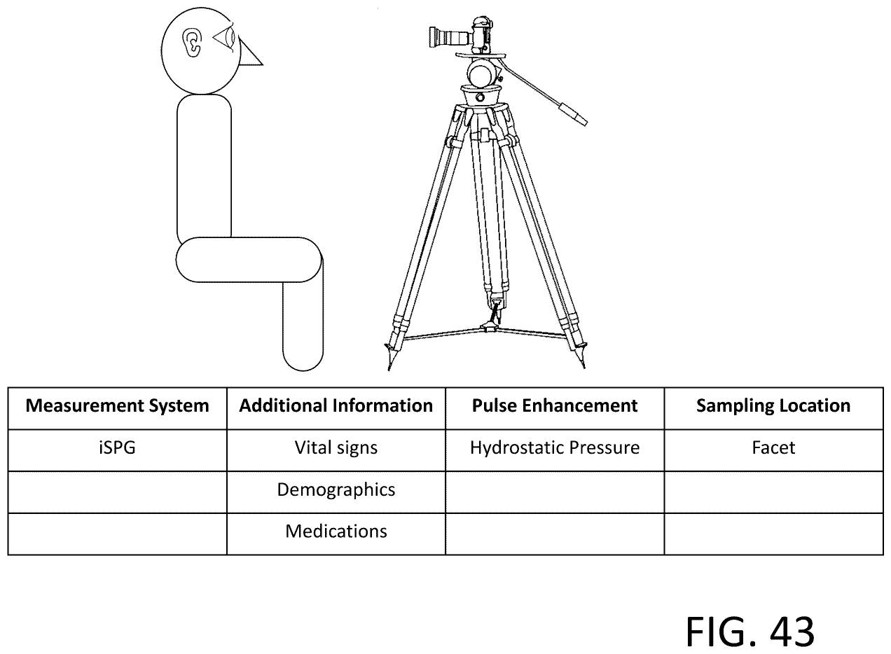

Face Speckle measurements. FIG. 43 illustrates pictorially a speckle imaging application on the face of the patient. In general imaging PPG or SPG works best on highly vascular areas such as the hands or face. Thus, the face represents a vascular-rich skin surface, with decreased transmural pressure due to a location above the heart. The work of Dunn et. al. examined the signal-to-noise characteristics of speckleplethysmographic (SPG) and photoplethysmographic (PPG) imaging by Monte Carlo simulations and in vivo measurements. They found that “SPG has a much larger SNR than PPG, which may prove beneficial for non-contact wide-field optical monitoring.” (Dunn, Cody E., et al. “Comparison of speckleplethysmographic (SPG) and photoplethysmographic (PPG) imaging by Monte Carlo simulations and in vivo measurements.” Biomedical optics express 9.9 (2018): 4307-4317.)

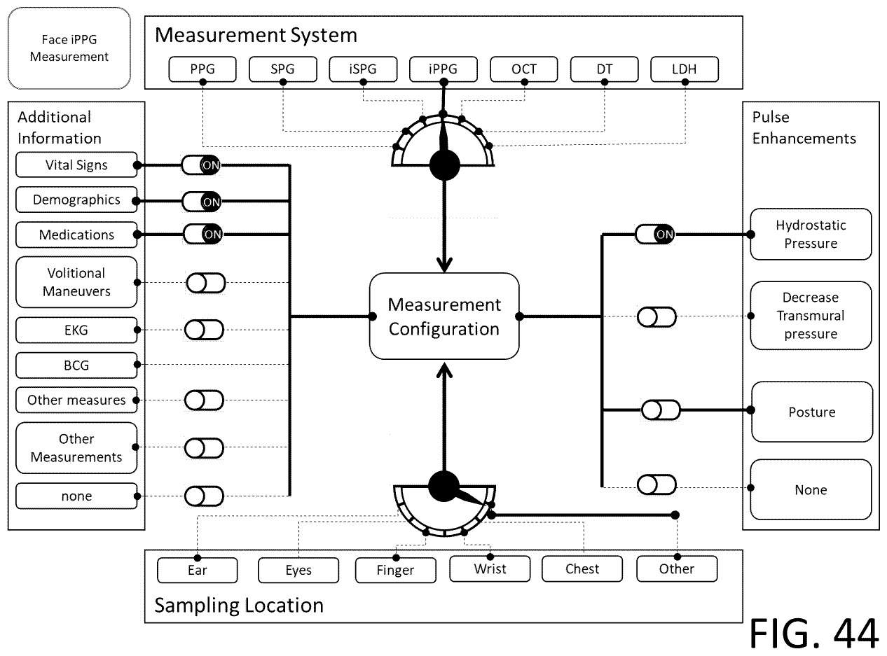

FIG. 43 illustrates a simple pictorial diagram of the measurement configuration and the key elements of the embodiment. FIG. 44 illustrates the measurement configuration used for the illustrated embodiment. The system uses speckle imaging as the measurement method on the face, by use of a camera or other optical system. The face is relatively close to the heart and is easily accessed for sampling. Additional information including vital signs, demographics, and medications are included and used by the analysis system.

The resulting SPG signals obtained from the face can be processed by the left ventricular outflow tract assessment system in multiple ways. Specifically, features can be extracted from the data stream, or the totality of video information used for analysis as a continuous data stream, or a combination of both. The left ventricular outflow tract assessment system then provides a measurement result which can be a simple probability score associated with a left ventricular outflow tract abnormality or a specific diagnosis and a severity score.

Carotid artery sampling. An additional embodiment (not illustrated) defines the peripheral sampling location as the carotid artery. The sampling location is close to the heart and easily accessible. The system can use speckle plethysmography as the measurement method to acquire an optical pulse plethysmogram with diminished sensitivity to vascular tone changes as the blood supply to the brain is maintained at constant levels. Additional information can be included in the analysis process as desired.

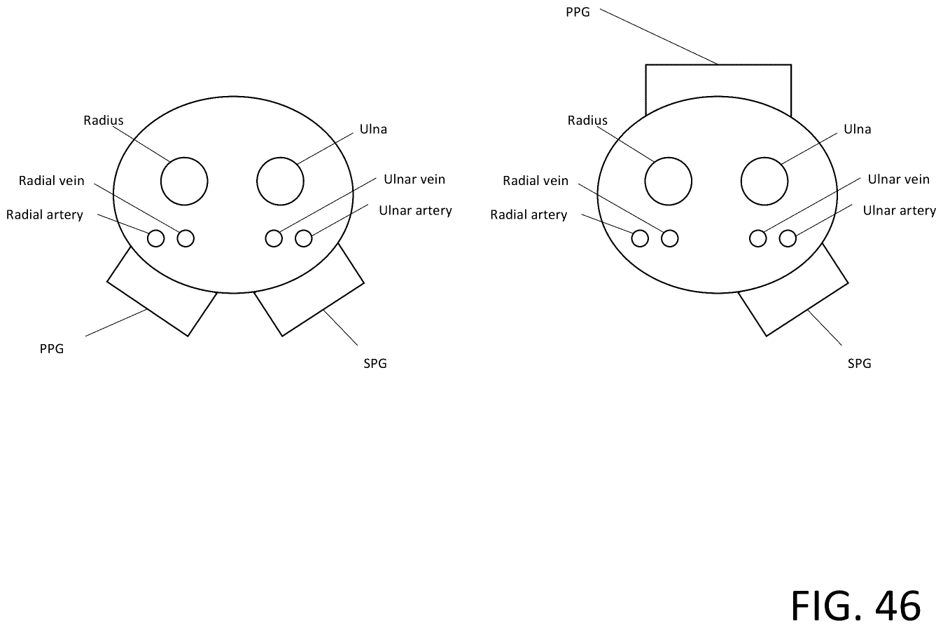

Combined SPG and PPG System. An additional proposed embodiment (FIG. 46) combines flow and volume measurements in a singular system. The system can be attached to the wrist, and SPG measurements can be obtained from the radial artery, while PPG measurements can be obtained from the back of the wrist or the ulnar artery. The combined system can use the information from volume changes and flow changes in the left ventricular outflow tract assessment system to provide classification information and quantitative results associated with disease status or measurement parameters such as aortic valve area. A similar dual sampling configuration can be used to sample the right and left carotid arteries.

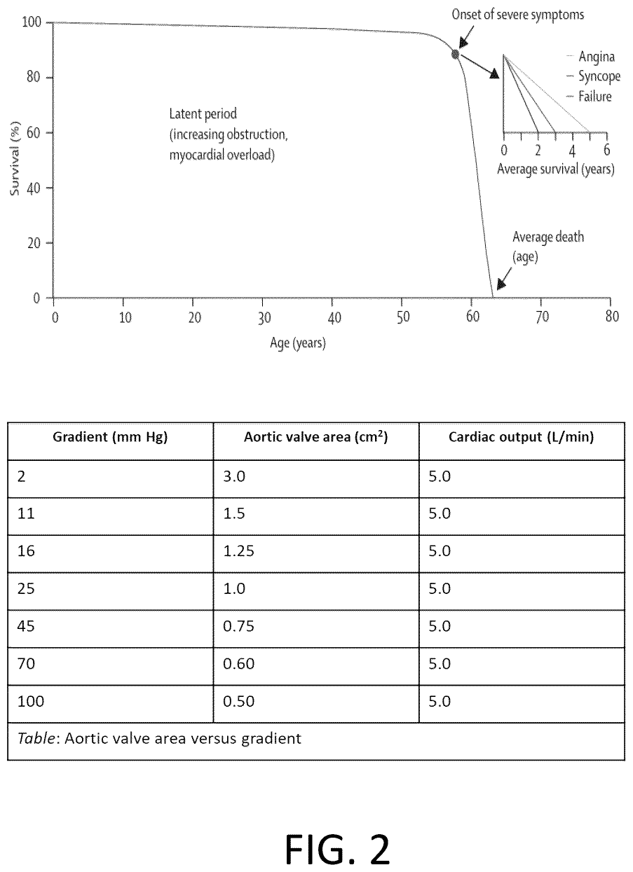

Screening and Monitoring Disease Progression. As illustrated in FIG. 2, patients can have a long latent asymptomatic period followed by a rapid demise. The work of Tastet et al. demonstrates that although the patient might report being asymptomatic cardiac damage is occurring, with 88% of patients having damage at the point of diagnosis. (Tastet, Lionel, et al. “Staging cardiac damage in patients with asymptomatic aortic valve stenosis.” Journal of the American College of Cardiology 74.4 (2019): 550-563.) Thus, yearly monitoring of patients over 50 years of age should occur for detection of outflow tract obstructions and the avoidance of cardiac damage during the long latent asymptomatic period. FIG. 27 is an illustration of this type of monitoring of two patients. The Y-axis shows the estimated change in aortic valvular area from a baseline measurement taken between age 50-59. The incidence of aortic stenosis is less than 0.5% in younger patients so it can serve as an effective baseline. (Eveborn, Gry Wisthus, et al. “The evolving epidemiology of valvular aortic stenosis. The Tromsø study.” Heart 99.6 (2013): 396-400.)

Patient #1 has no evidence of aortic stenosis. Patient #2 begins to develop stenosis with a significant decrease in aortic valve area. At age 70, the patient has an estimated aortic valve area of 50% the original baseline and has their valve replaced with a return to pre-stenosis function.

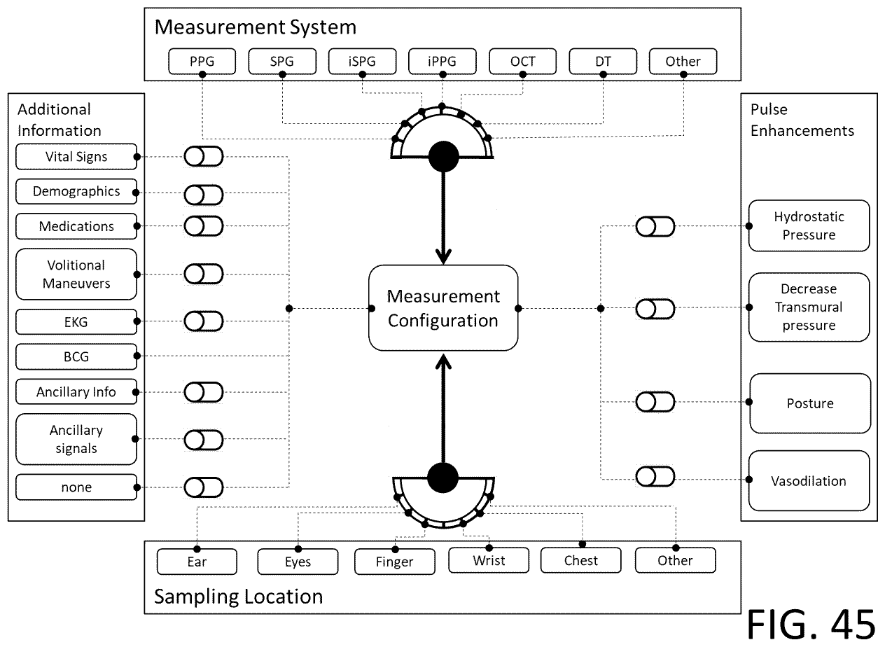

Additional Embodiments. Additional measurement combinations are contemplated as easily visualized in FIG. 45. The figure illustrates that various combinations can be used to make determinations with respect to left ventricular outflow tract obstruction. However, some configurations may be able to detect only the presence of an abnormality, while others may be able to deter the type and degree of obstruction present. The measurement configurations presented are for illustrative purposes and can be tailored to meet user objective in terms of performance, expense, and data acquisition difficulty.

Implementation. Methods and apparatuses involving analysis, models, and controls in the context of the present invention can be implemented using one or more programmable data processors such as computers, which are programmed for use in the system. Traditionally, a computer program consists of a finite sequence of computational instructions or program instructions. It will be appreciated that a programmable apparatus (i.e., computing device) can receive such a computer program and, by processing the computational instructions thereof, produce a further technical effect.

A programmable apparatus can include one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of a special-purpose computer, programmable data processing apparatus, processor, processor architecture, and so on.

A computer can include a computer-readable storage medium that can be internal or external, removable, and replaceable, or fixed.

Embodiments of the systems as described herein are not limited to applications involving conventional computer programs or programmable apparatuses that run them. It is contemplated, for example, that embodiments of the invention as claimed herein can include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) can be utilized. The computer readable medium can be a computer readable signal medium or a computer readable storage medium. A computer readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium can include a propagated data signal with computer readable program code embodied therein, for example, in baseband or as part of a carrier wave. Such a propagated signal can take any of a variety of forms, including, but not limited to, electro-magnetic, optical, or any suitable combination thereof. A computer readable signal medium can be any computer readable medium that is not a computer readable storage medium and that can communicate, propagate, or transport a program for use by or in connection with an instruction execution system, apparatus, or device.

Program code embodied on a computer readable medium can be transmitted using any appropriate medium, including, but not limited to, wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof can be implemented as parts of a monolithic software structure, as standalone software modules, or as modules that employ external routines, code, services, and so forth, or any combination of these. All such implementations are within the scope of the present disclosure.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. It is possible to modify or customize general-purpose systems to be used with programs in accordance with the teachings herein, or it might prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages can be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware specialized through computer instructions; and so on-any and all of which can be generally referred to herein as a “circuit,” “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations can depict a step, or group of steps, of a computer-implemented method. Further, each step can contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

The present invention has been described in connection with various example embodiments. It will be understood that the above description is merely illustrative of the applications of the principles of the present invention, the scope of which is to be determined by the claims viewed in light of the specification. Other variants and modifications of the invention will be apparent to those skilled in the art.

### Demonstration of Feasibility on In Silico Data

### Demonstration Objective

The objective was to create a large and representative set of in silico data over a representative patient population aged 50 to 75 years with significant physiological variances present, to include the development of aortic stenosis. Both volume or photo plethysmograms and speckle or flow plethysmograms were created. The resulting data was processed to determine the ability to reliably predict aortic valve area.

### Data Simulation Overview

The aim of this study was to create a dataset of simulated pulse wave signals that represents a real population of subjects aged 50 to 75 years under normal physiological conditions and with varying degrees of aortic valve stenosis (AVS). This was achieved by combining existing verified methodologies, including a-state-of-the-art heart model to enable the implementation of AVS progression. The dataset was developed with the criteria: 1) extension of an existing 1D blood flow model with the inclusion of a zero-dimensional (0D) four-chamber heart model; 2) verification of the new model to ensure it accurately represents in vivo data; and 3) creation of a database of pulse waves representative of an initially healthy population that subsequently undergoes various levels of AVS.

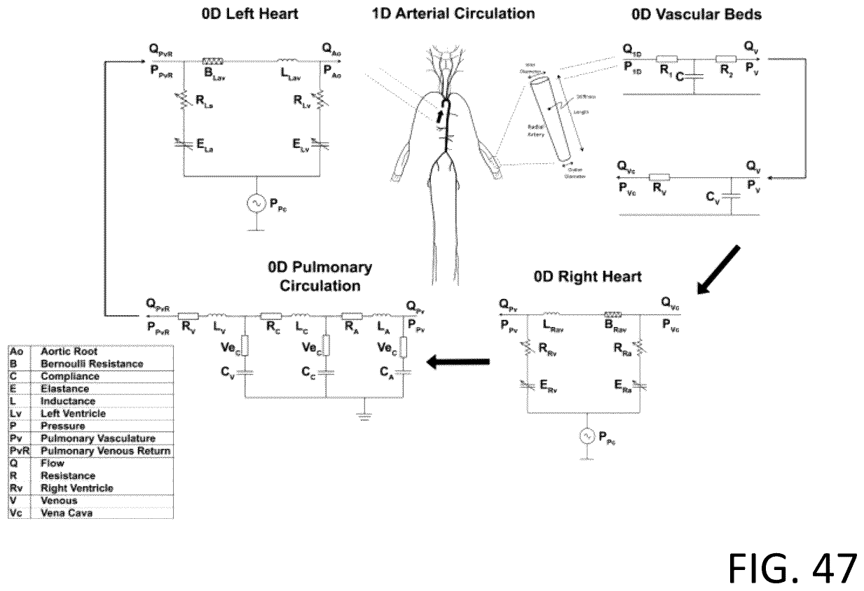

Pulse wave signals for blood pressure, photoplethysmography (PPG), blood flow velocity, and blood flow rate were simulated using 1D blood flow modelling in the larger systematic arteries of the thorax, limbs, and head, as originally described in the dataset by Charlton et al. This model was further extended with the addition of a zero-dimensional (0D) four-chamber heart model coupled to the 1D arterial circulation model. For data generation a closed loop 1D/0D blood flow model of the entire cardiovascular system (FIG. 47) was used to generate pulse wave signals in a data set comprising thousands of virtual subjects. (Charlton et al. “Modeling arterial pulse waves in healthy aging: a database for in silico evaluation of hemodynamics and pulse wave indexes.” American Journal of Physiology-Heart and Circulatory Physiology (2019) 317, H1062-H1085).

The generation of the in silico pulse waves dataset under normal physiological conditions followed several key steps, all of which were based on the methodology outlined in Charlton et al. First, a 25-year-old baseline subject was simulated, generating pulse wave signals at common measurements sites that qualitatively matched in vivo signals from a real 25-year-old subject. Second, the clinical literature was reviewed to determine how various cardiovascular parameters of the 25-year-old baseline model change with age. Third, baseline subjects at ages 50-75 years in five-year increments were simulated using the age-specific mean values for all parameters described in Step 2. Fourth, variability in pulse wave signals within each age group was accounted for by adjusting cardiovascular properties in combination with each other by ±1standard deviation (SD) from their age-specific mean values. For each virtual subject, 10 different levels of AVS were then simulated. The inclusion of the heart model tripled the simulation time for each subject (approximately 30 minutes) compared to the open-loop simulations in Charlton et al.

The initial step involved the recalibration of the baseline 25-year-old subject, as presented in Charlton et al., along with the incorporation of the 0D four-chamber heart model (FIG. 47). The cardiac parameters from the original dataset were no longer applicable, as the inflow boundary condition that previously prescribed them was removed from the new model. Arterial, vascular bed, and blood properties, heart rate (HR) were input parameters. Stroke volume (SV) and left ventricular ejection time (LVET) were calculated during the simulation, by solving physical equations that describe ventricular-aortic coupling. A parameter sensitivity analysis was carried out to determine the impact of several cardiac properties (SV, LVET, and elastance and valve functions) on the simulated PWs. Based on this analysis, the cardiac properties for the 25-year-old subject were fine-tuned by adjusting the total blood volume, valve opening times, and elastance from the values provided by Mynard and Smolich, to align the model with the PW-based hemodynamic indices produced by the original model. (Mynard, J. P., Smolich, J. J., 2015. One-dimensional haemodynamic modeling and wave dynamics in the entire adult circulation. Annals of Biomedical Engineering 43, 1443-1460).

After successfully calibrating the baseline 25-year-old subject, baseline subjects between ages 50 to 75 years old at five-year intervals were created. This age bracket was chosen due to its strong association with AVS and valvular replacement. In Charlton et al., calibration of additional baseline subjects was achieved by conducting a review of the clinical literature to identify variations in the following cardiovascular properties with chronological age (mean±1SD): HR, SV, LVET, peak aortic flow time, reverse flow volume, arterial lengths, arterial diameters, arterial wall stiffness (measured by pulse wave velocity), arterial wall viscosity, systemic vascular resistance, systemic vascular compliance, and blood density and viscosity. Mean values for all these arterial, vascular bed, and blood properties were specified as input parameters for our baseline subjects, but HR was the only cardiac parameter that can be directly prescribed. Heart parameters defining the elastance and valve function were adjusted to closely mimic the age variations in the mean values of the following parameters used in Charlton et al.: SV, LVET, peak aortic flow time, blood pressure and reverse flow volume.

Within each age group, variability in pulse wave signals was simulated by modifying five cardiovascular properties from their corresponding values for the group's baseline subject: HR, SV, arterial diameter, pulse wave velocity, and mean arterial pressure. A sensitivity analysis revealed that these five properties had the most significant impact on the simulated pulse waves within an age group. These properties were varied in combination with each other by ±1SD from their age-specific mean values. This resulted in 3⁵=243 for each age group and a total of 243×6=1,458 subjects under normal physiological conditions.

AVS was simulated by decreasing the orifice area of the aortic valve in the 0D left heart compartment. Under normal physiological conditions, each virtual subject had a baseline orifice area of 4.9 cm² which was decreased within the range of 3.0 cm² to 0.5 cm², in ten 0.25 cm² increments, to simulate between mild to severe AVS, according to clinical guidelines. This new range of orifice areas extended the 1,458 virtual subjects under normal physiological conditions to a total of 1,458×11=16,038 subjects; 14,580 of which had some level of AVS present.

The pulse wave database was verified through several comparisons with in vivo data. First, the shapes of simulated pulse waves for virtual subjects of different ages under normal physiological conditions were compared with in vivo pulse waves at different ages (Flück et al., “Effects of aging on the association between cerebrovascular responses to visual stimulation, hypercapnia, and arterial stiffness. Frontiers in Physiology (2014) 5, 49.), normotensive subjects during screening for hypertension (including aortic root pressure PWs estimated using a transfer function) (Li et al., “Forward and backward pressure waveform morphology in hypertension.” Hypertension (2017) 69, 375-381.), and the Vortal dataset (Charlton et al., “An assessment of algorithms to estimate respiratory rate from the electrocardiogram and photoplethysmogram.” Physiological Measurement (2016) 37, 610. Charlton et al., “Extraction of respiratory signals from the electrocardiogram and photoplethysmogram: technical and physiological determinants.” Physiological Measurement (2017) 38, 669). Second, the hemodynamic characteristics of the simulated PWs (also under normal physiological conditions) were compared with the in vivo hemodynamic values reported in McEniery et al. Third, the simulated flow velocities at the aortic root and resulting Reynolds numbers with increasing AVS severity were compared to corresponding in vivo data from real 4 subjects, with both normal and diseased aortic valves, from several studies. (McEniery, et al. Normal vascular aging: differential effects on wave reflection and aortic pulse wave velocity: the Anglo-Cardiff Collaborative Trial (ACCT). Journal of the American College of Cardiology (2005) 46, 1753-1760.)

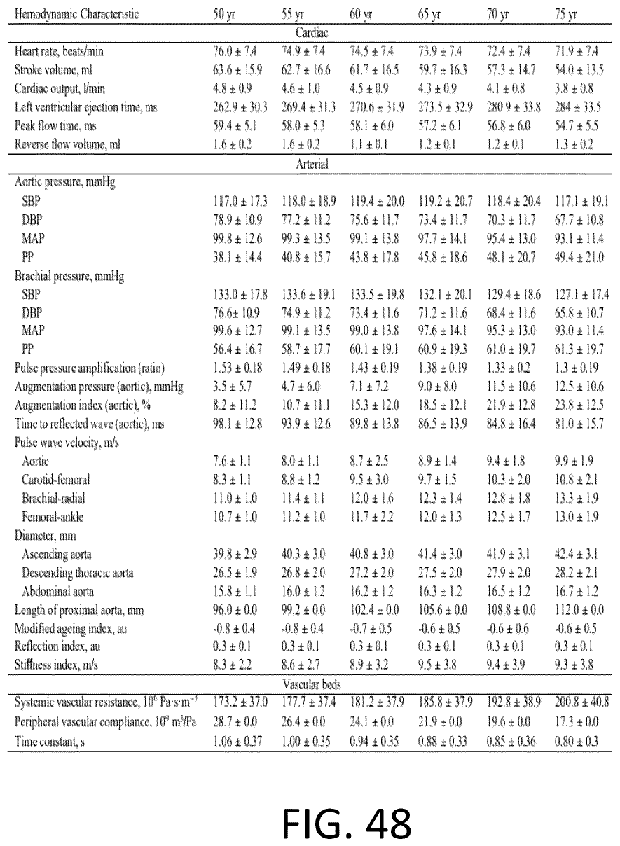

FIG. 48 Summarizes the hemodynamic characteristics of all healthy subjects, demonstrating a wide range of cardiovascular physiology exhibited by individuals in the database. This variation is observed across the entire age range and within each age group.

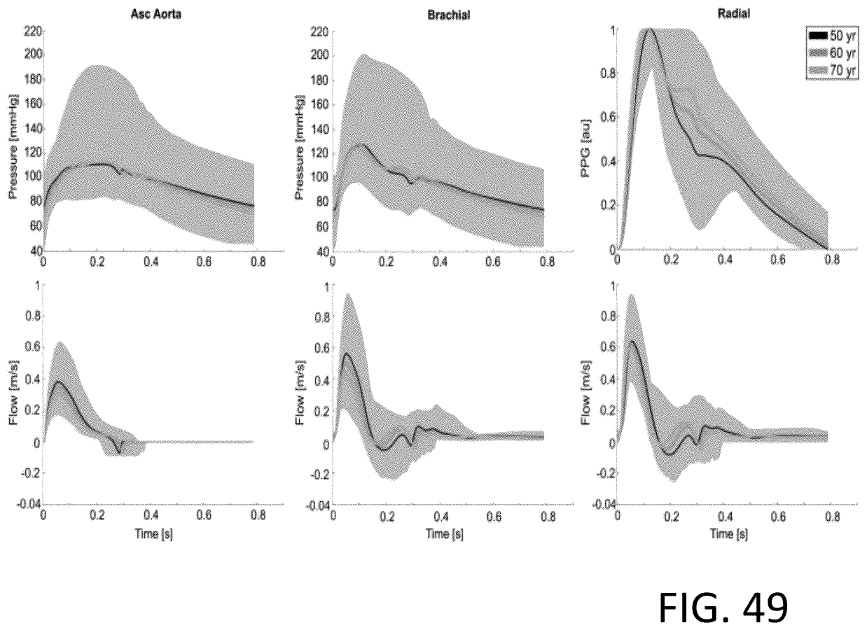

FIG. 49 further illustrates the substantial variability in pulse waveforms across all healthy subjects in the database. Some parameters, such as HR and proximal aortic length, were prescribed to the model and therefore predetermined.

In contrast, all other cardiac characteristics and many of the hemodynamic pulse wave parameters were not directly prescribed but were derived from simulated pulse waves. These derived parameters include stroke volume, peak flow time, systolic blood pressure, pulse pressure (PP), and pulse wave velocities. Notably, these derived parameters exhibited significant changes with age, highlighting that the different input parameter values prescribed at each age resulted in realistic variations in pulse wave shapes, mirroring in vivo observations.

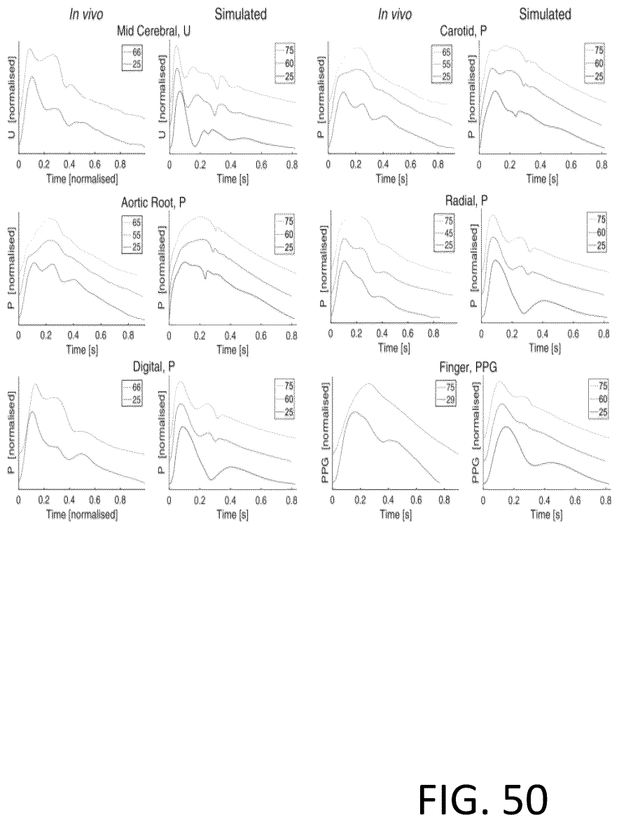

FIG. 50 compares a selection of simulated pulse waves with pulse waves from the literature. It displays pulse waves from both the pulse wave database (simulated) and the literature (in vivo) for young, middle-aged, and elderly subjects. The shapes of the simulated pulse waves generated using the closed-loop model closely align with those found in Charlton et al.'s dataset, changing with age in a manner similar to the in vivo pulse waves: 1) the amplitude of the secondary systolic peak in middle cerebral flow velocity pulse waves increases with age; 2) the augmentation in the secondary systolic peak of the carotid and ascending aorta pressure pulse waves also increases with age; and 3) the diastolic peak of the finger PPG pulse wave disappears with age.

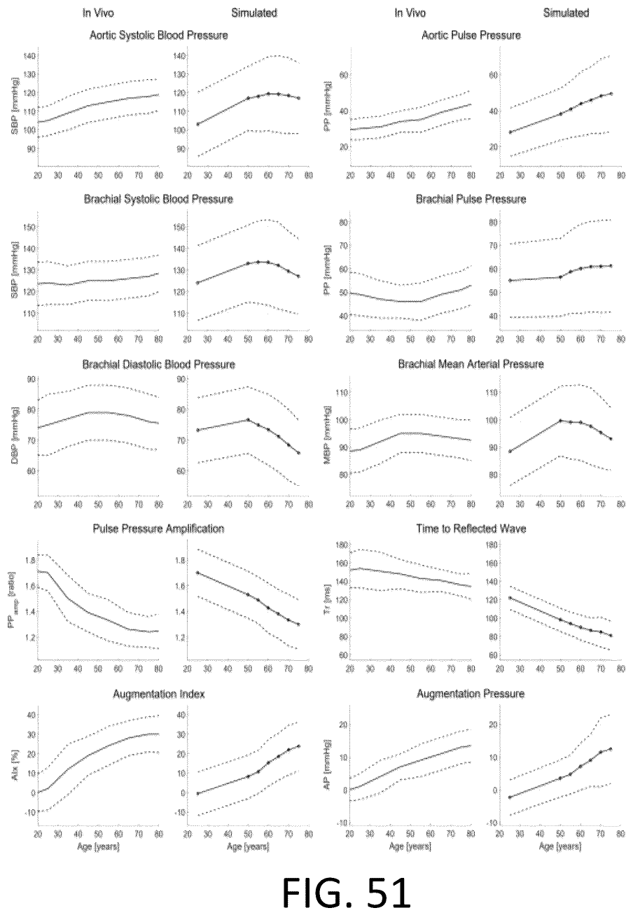

FIG. 51 compares the hemodynamic characteristics of the simulated PWs, including some from FIG. 49, with those from the literature. It also includes data for the 25-year-old baseline subject to illustrate the overall trend across a wider age scale. The changes observed with age were mostly consistent between the literature (left-hand plots) and the simulated (right-hand plots) characteristics: 1) aortic pulse pressure (PP) increases with age; 2) brachial PP increases from ages 50 to 75; 3) brachial diastolic and mean arterial pressures increase in young subjects but decrease from ages 50 to 75; 4) PP amplification decreases with age but pressure augmentation increases with age (AIx and AP); and 5) the time to the return of the reflected pressure wave (Tr) decreases with age. However, in silico aortic systolic pressure dropped slightly from age 65, brachial diastolic pressure exhibited a steeper decrease from age 50 onwards, and Tr also showed a sharper decline, resulting in in silico values lower than in vivo values. Overall, the hemodynamic characteristics of the simulated PWs demonstrated similar trends and, in most cases, similar absolute values, to those reported in the literature.

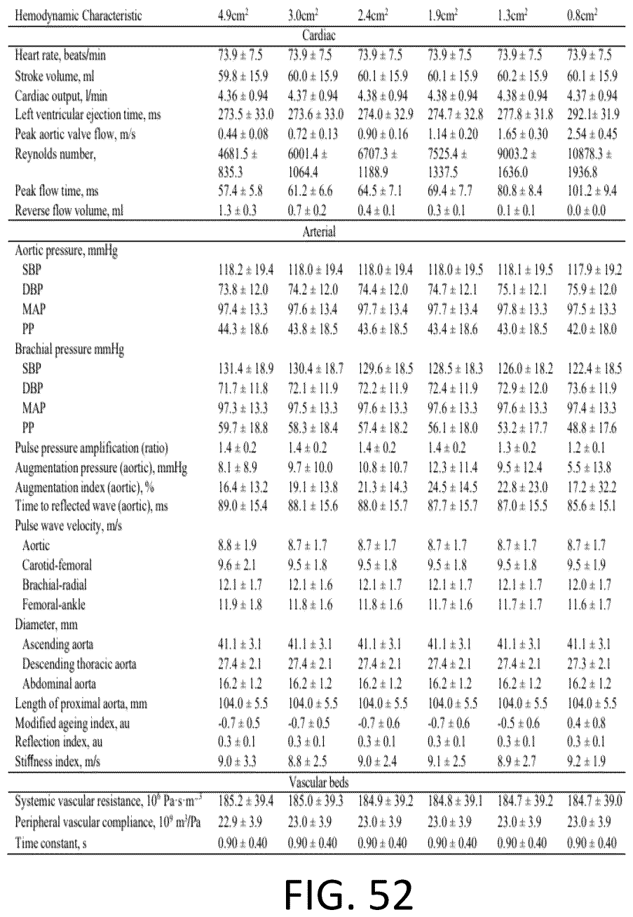

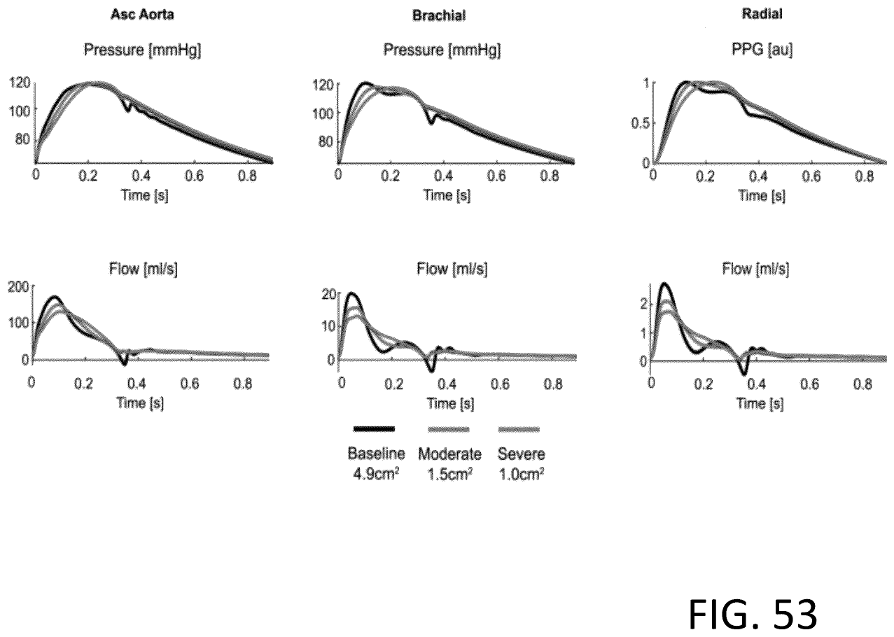

FIG. 52 summarizes the hemodynamic characteristics of all 75-year-old subjects. As the orifice area decreased (moving from left to right columns), the time and magnitude of the aortic peak flow increased, while the reverse aortic flow volume decreased. Therefore, AVS led to a more rounded flow wave in the ascending aorta, with a smaller peak value and reduced reverse flow (FIG. 53). This resulted in a less pronounced rate of increase in early systolic flow, which, in turn, led to a less pronounced increase in early systolic aortic pressure and a longer time from start of systole to aortic systolic pressure (FIG. 53). This relationship is explained by the direct connection between changes in early systole pressure and flow through the water hammer equation (Vennin et al., 2021).

These changes in aortic pulse waves resulted in significant variations in peripheral pulse wave morphology due to AVS (FIG. 53) the brachial PP decreased, primarily driven by a reduction in brachial systolic blood pressure, leading to a decrease in PP amplification, in agreement with in vivo data (Nielsen et al., 2016); 2) the peak and mean flows in the brachial and radial arteries decreased; and 3) the time from pulse onset to peak PPG increased at the wrist, as well as the diastolic peak/inflection point. PP amplification is determined by the rate of increase/decrease in blood flow, increasing with a faster rate in early systole and a faster decrease rate in late systole (Flores Gerónimo et al., 2021). Consequently, the smaller rates observed in the ascending aorta with AVS led to smaller PP values in the periphery. The more rounded and lower-amplitude aortic flow waves propagated throughout the arterial network, as pulse wave propagation is governed by a system of hyperbolic partial differential equations (Alastruey et al. 2023). In such systems, boundary conditions significantly influence calculated variables within the computational domain.

The hemodynamic mechanisms described above also impacted the peripheral PPG waveform, as there is a direct relationship between PPG and pressure wave morphology (Millasseau et al., 2000). Notably, the increased time from pulse onset to peak aortic pressure was transmitted to peripheral pressure waveforms and, subsequently, PPG waveforms. The reduced peripheral PPs contributed to the augmentation of the PPG diastolic peak/inflection point.

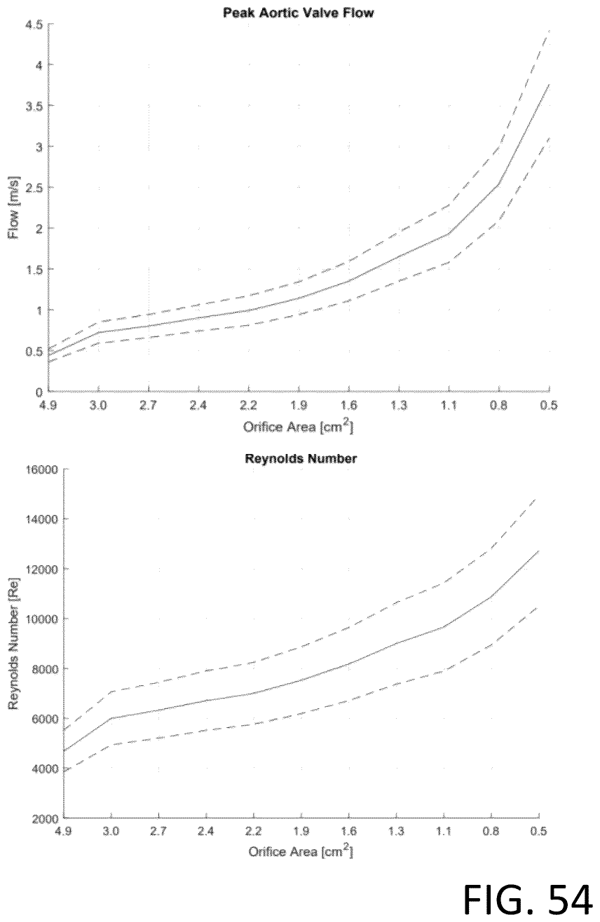

As the level of AVS increased, there was a corresponding rise in the Reynolds number calculated from the flow across the aortic valve into the aorta (FIG. 52) (FIG. 54). This aligns with in vivo studies that have reported turbulent flow in the ascending aorta of AVS subjects with peak Reynolds numbers ranging from 5,700 to 8,900 (Stein and Sabbah, 1976).

We have created a database comprising 16,038 virtual subjects, which accurately represents a real population of individuals aged 50 to 75 under normal physiological conditions, encompassing varying degrees of AVS. For each subject, central and peripheral pulse wave signals for blood pressure, PPG, blood flow velocity, and blood flow rate were simulated at common measurements sites using a closed loop 1D/0D blood flow model of the entire cardiovascular system. The pulse waves within the database exhibit age-related changes in hemodynamic parameters and pulse wave morphology that closely mimic in vivo data from individuals of the same age. Notably, centrally observed changes in pulse wave morphology were amplified by transmission through the arterial network to the periphery. This dataset was used to evaluate the noninvasive, direct quantification of aortic valve orifice area and is well-suited for machine learning approaches due to its large size and comprehensive coverage of variance in cardiovascular properties.

### Data Graphs

For visualization purposes and to show the pulse wave differences present in the in silico data, three specific plots of the data from two different measurement locations are provided in the figures. The figures are as follows:

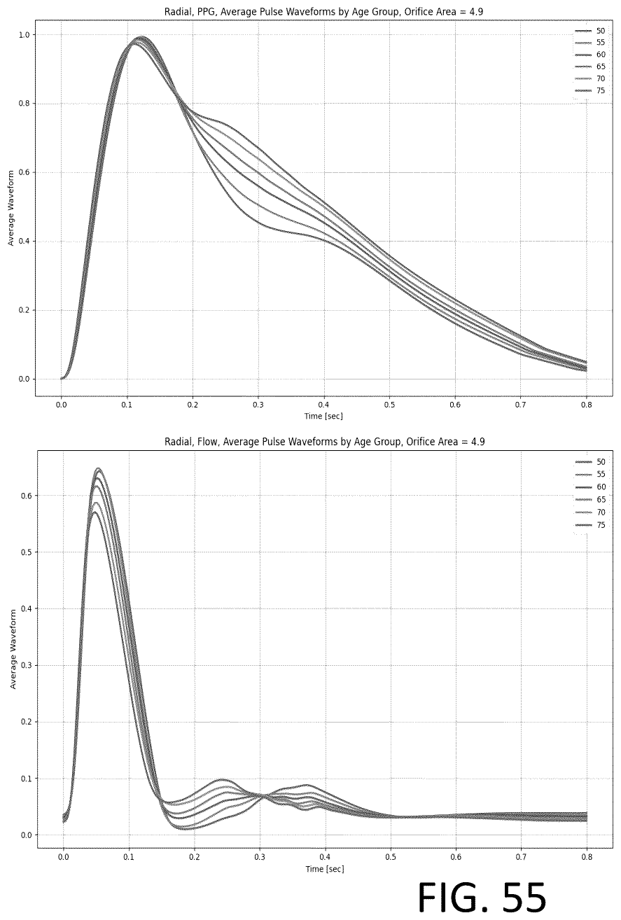

FIG. 55 plots SPG and PPG pulse waves at the radial artery sampling location with variance in age between 50 and 75. The aortic valve area was maintained at 4.9 cm², and cardiovascular properties: HR, SV, arterial diameter, pulse wave velocity, and mean arterial pressure were at mean values for each age group.

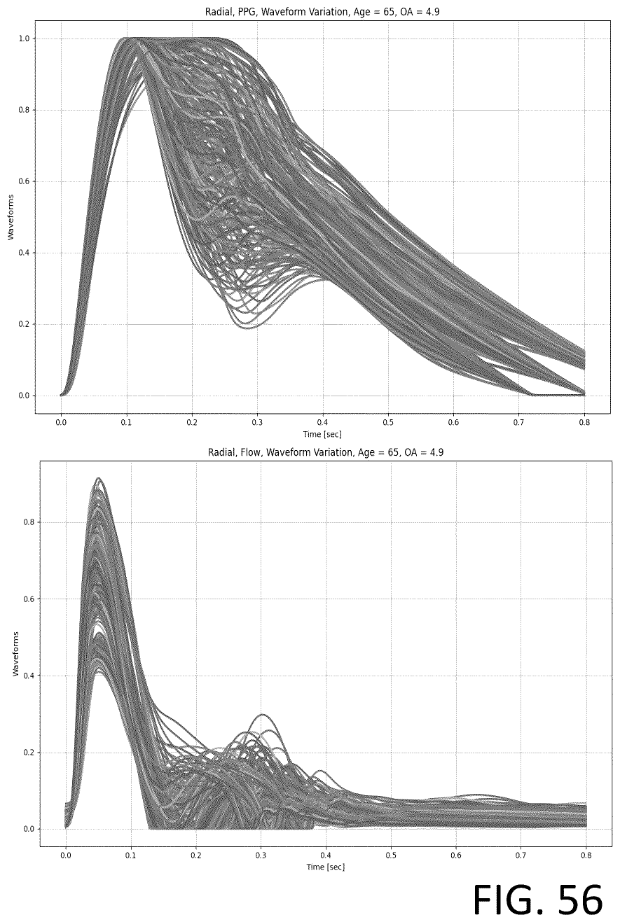

FIG. 56 plots SPG and PPG pulse waves at the radial artery sampling location with variance of cardiovascular properties: HR, SV, arterial diameter, pulse wave velocity, and mean arterial pressure were at mean age values (mean±1SD). The aortic value area was maintained at 4.9 cm² and the age was maintained at 65 years.

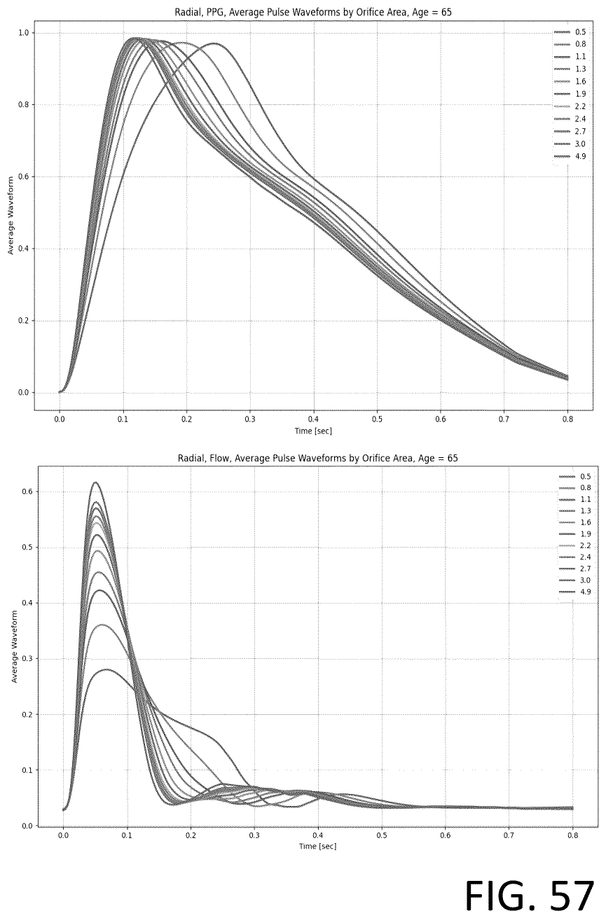

FIG. 57 plots SPG and PPG pulse waves at the radial artery sampling location with aortic valve areas representing a baseline orifice area of 4.9 cm² and reduced orifice areas within the range of 3.0 cm² to 0.5 cm², in ten 0.25 cm² increments. The age was maintained at 65 years and cardiovascular properties: HR, SV, arterial diameter, pulse wave velocity, and mean arterial pressure were at the mean age values.

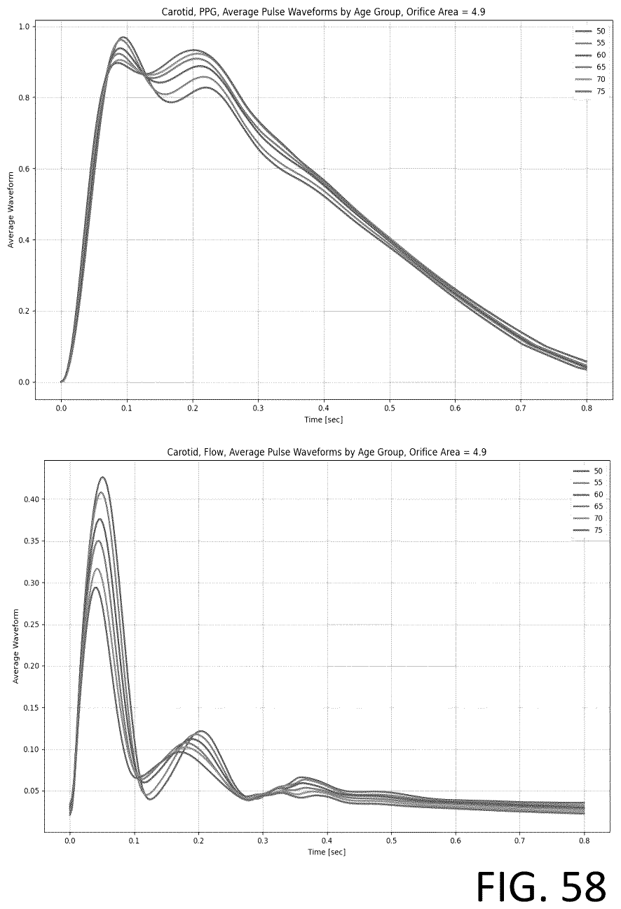

FIG. 58 plots SPG and PPG pulse waves at the carotid artery sampling location with variance in age between 50 and 75 The aortic value area was maintained at 4.9 cm² and cardiovascular properties: HR, SV, arterial diameter, pulse wave velocity, and mean arterial pressure were at mean values for each age group.

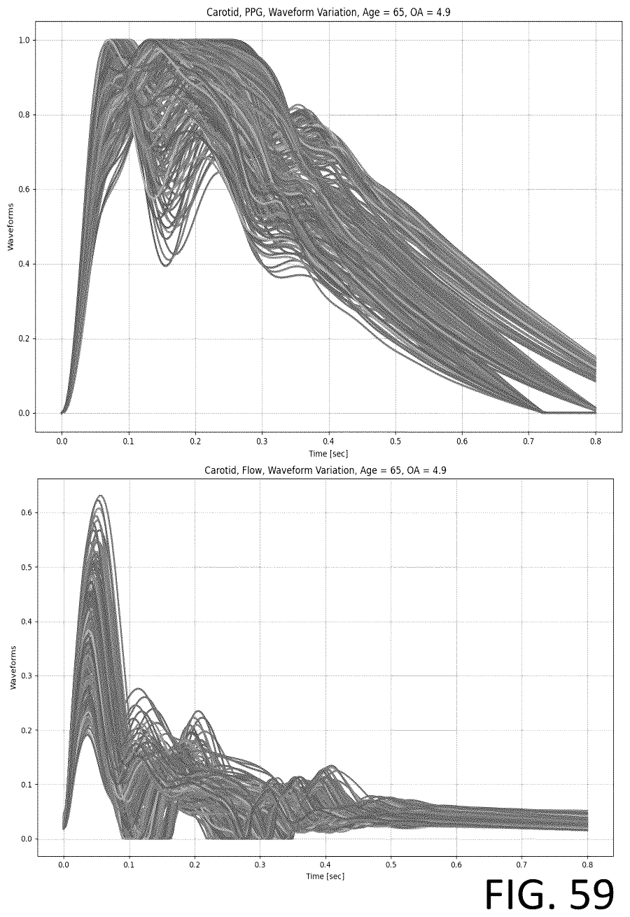

FIG. 59 plots SPG and PPG pulse waves at the carotid artery sampling location with variance of cardiovascular properties: HR, SV, arterial diameter, pulse wave velocity, and mean arterial pressure were at mean age values (mean±1SD). The aortic value area was maintained at 4.9 cm² and the age was maintained at 65 years.

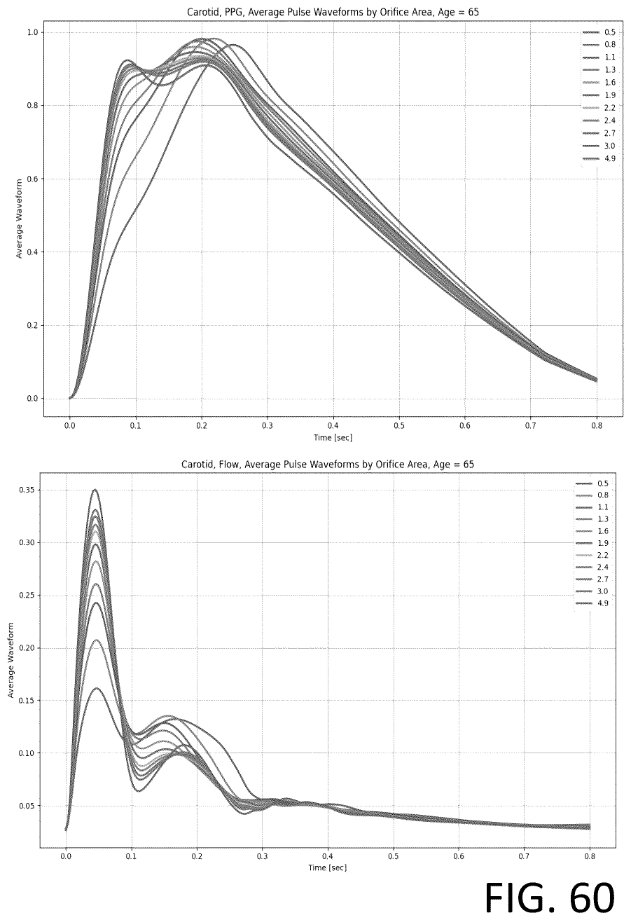

FIG. 60 plots SPG and PPG pulse waves at the carotid artery sampling location with aortic valve areas representing a baseline orifice area of 4.9 cm² and reduced orifice areas within the range of 3.0 cm² to 0.5 cm², in ten 0.25 cm² increments. The age was maintained at 65 years and cardiovascular properties: HR, SV, arterial diameter, pulse wave velocity, and mean arterial pressure were at mean age values.

### Analysis Overview

The mapping function that converts measurements to an estimate of the area of the aortic valve or other measure of the severity of aortic stenosis can be a linear or nonlinear function. In one exemplary embodiment the mapping function is a nonlinear function comprised of a neural network (NN). In one exemplary embodiment a waveform comprised of some or all of a single cardiac pulse or a series of cardiac pulses from a single site pass into a recurrent neural network (RNN). The waveform may be derived from measurement modalities of SPG, PPG, flow, or pressure. The RNN may be one of a Long-Short Term Memory (LSTM), Gated Recurrent Unit (GRU), or other RNN as known in the art. Alternatively, the waveform may be initially processed with a neural network architecture like a Transformer Encoder (TE) or other NN architecture as known in the art for operating on sequential data. In one exemplary embodiment multiple RNNs, TEs, or other sequential processing architectures may be mixed and or combined in multiple stages that are sequential, parallel, or a combination of the two. The portion of the NN that receives and operates on a waveform, or a sequence of waveforms shall be referred to herein as “input NN.”

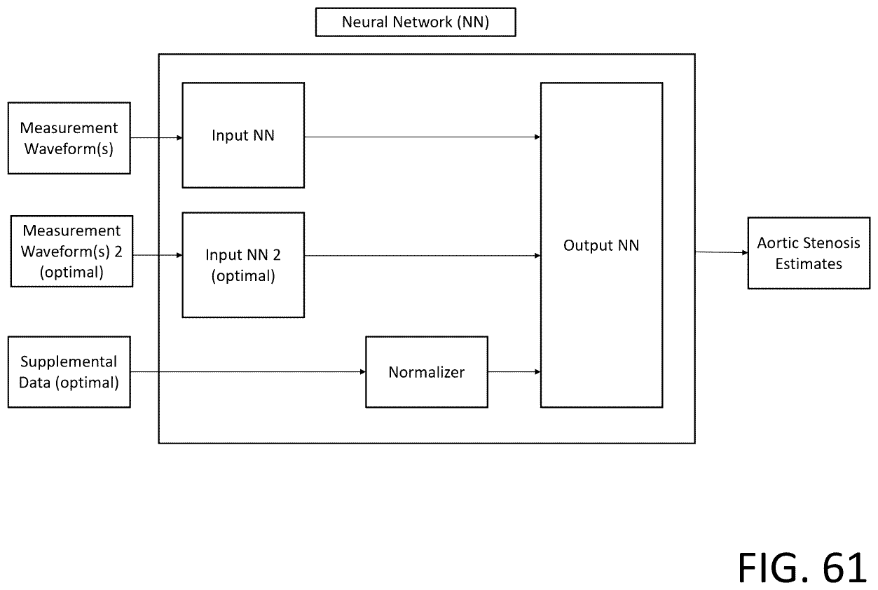

In one exemplary embodiment, waveforms from a single measurement modality from two or more sites on the body may be inputs into the mapping function. The waveforms from a plurality of sites may be processed sequentially or in parallel. In one exemplary embodiment, the initial processing by the input NN will be performed in parallel such that the initial processing of the waveform from the first body site will be performed by a different input NN than the processing of the waveform from a second body site. In another exemplary embodiment, the waveforms from the multiple body sites may be processed as a multidimensional signal into input NN. FIG. 61 is a pictorial representation of a NN block diagram. In some cases, one or more of the waveforms so processed may require interpolation and/or resampling in order to align the waveforms.

Similarly to the use of multiple waveforms from two or more body sites, multiple waveform-measurement modalities may be used as inputs into the input NN. These waveform measurements may come from substantially the same site on the body or from different body sites. As with waveforms of the same measurement modality from multiple body sites, waveform measurements from multiple modalities may be processed by the input NN in series, in parallel, or as multidimensional input signals. The latter form of inputs may require interpolation and/or resampling of one or more waveforms. In some embodiments, waveforms from multiple body sites and multiple measurement modalities may be combined sequentially, in parallel, or as multidimensional input signals.

In one exemplary embodiment, the outputs from the input NN are processed by other NN components that reduce the outputs of the input NN to one or more values that are related to the estimate of the aortic valve area. This portion of the NN is referred to herein as the “output NN.” The output NN may comprise convolutional layers, partially connected layers, transformer decoders, or other NN components and architectures as known in the art. In one embodiment, the output NN comprises one or more fully connected NN layers.

In one exemplary embodiment, one or more supplemental values may be used as input signals in addition to the one or more waveforms. These supplemental values may be comprised of one or more of age, gender, weight, heart rate, cardiac stroke volume, left ventricular ejection time, one or more blood-pressure measurements, one or more pulse-width velocity measurements, and others of the sort. The supplemental values may be appended to the waveform(s) or otherwise processed by the input NN. In some embodiments the supplemental values may be appended to the output of the input NN before the signal are processed by the output NN. In a preferred embodiment, the supplemental values are normalized by batch normalization or other techniques known in the art in order to make the supplemental signal values have comparable ranges as the values derived from the waveform(s). In some embodiments, the final output of the NN maybe be restricted to physiological limits by the use of sigmoid components, TanH components, numerical clipping components or other components of the sort as known in the art.

During training of the NN, drop-out components and/or other components may be included in the NN architecture in order to regularize the training, as known in the art. The results presented in this document were generally developed using a cross-validation methodology in which a random selection of samples were held out and the remaining samples were used to train the NN. The trained NN was then used to predict on the held-out samples. This process was repeated multiple times such that every sample was held out and used to generate one prediction result. Most often, a three-fold cross-validation was used for the results presented herein.

### Results Overview

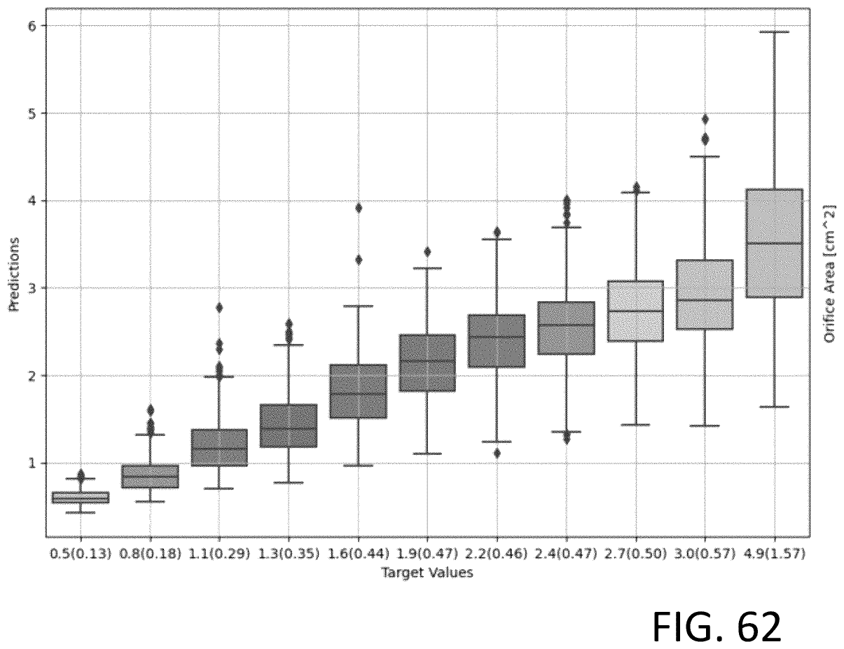

FIG. 62 illustrates the outcomes of the analysis for flow predictions utilizing the simulated waveforms at the radial artery. The prediction model is also supplemented with age data. Representative noise, approximately 10%, was incorporated. The box and whisker plot portrays target aortic valve area on the x-axis and corresponding predictions on the y-axis, with the Root Mean Square (RMS) error indicated next to each target value along the x-axis. The results showcase a notable performance, with RMS errors ranging from 0.13 for an aortic valve area of 0.5 cm² to 1.57 for an area of 4.9 cm². Of particular significance is the detection capability around 2.2 cm², where aortic stenosis often evades auscultation but can be identified with an impressive RMS error of 0.46.

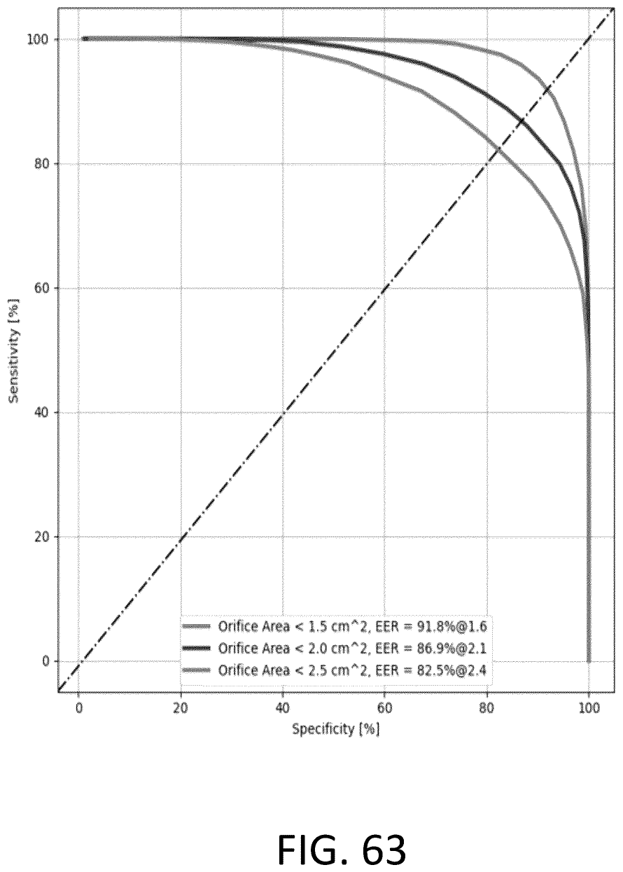

FIG. 63 is a plot of the true positive rate (Sensitivity) against the false positive rate (Specificity) for different threshold values. Sensitivity represents the proportion of true positive predictions correctly identified by the model while Specificity indicates the proportion of true negative predictions correctly identified. FIG. 63 is the Sensitivity versus Specificity for the flow predictions at the radial artery with age added as supplemental data with representative noise, approximately 10%, included. The equal error rate (EER) at an aortic valve area of 1.5 cm²is 91.8%. For aortic valve areas of 2.0 cm² and 2.5 cm², the EER are 86.9% and 82.5% respectively.

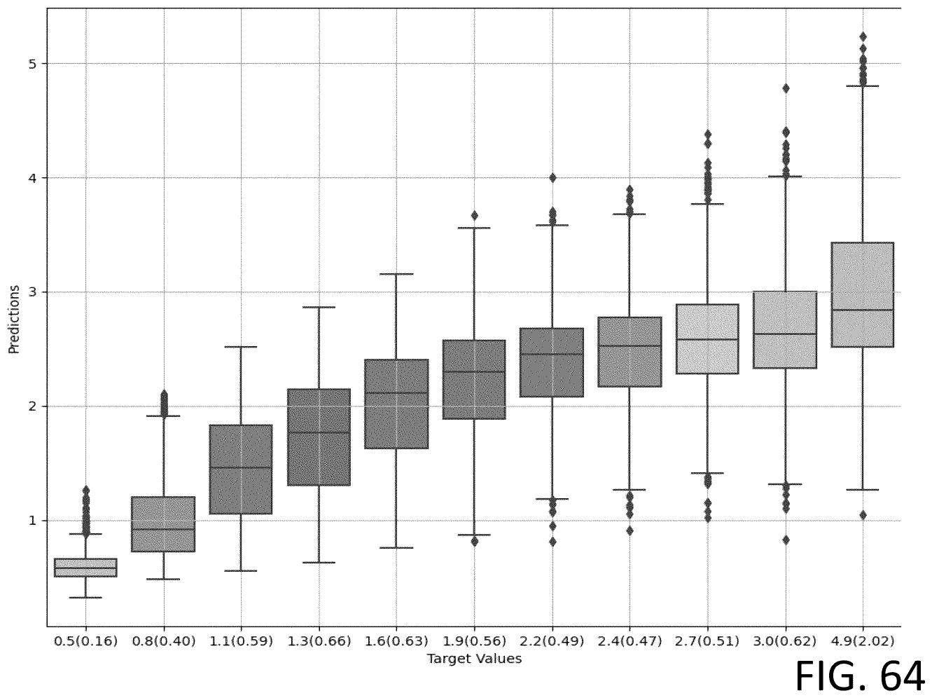

FIG. 64 illustrates the outcomes of the analysis for volume (ppg) predictions utilizing waveforms supplemented with age data at the carotid artery. Representative noise, approximately 10%, was incorporated. The box and whisker plot portrays target aortic valve area on the x-axis and corresponding predictions on the y-axis, with the Root Mean Square (RMS) error indicated next to each target value along the x-axis. The results showcase a notable performance, with RMS errors ranging from 0.16 for an aortic valve area of 0.5 cm² to 2.02 for an area of 4.9 cm². Of particular significance is the detection capability around 2.2 cm², where aortic stenosis often evades auscultation but can be identified with an impressive RMS error of 0.49.

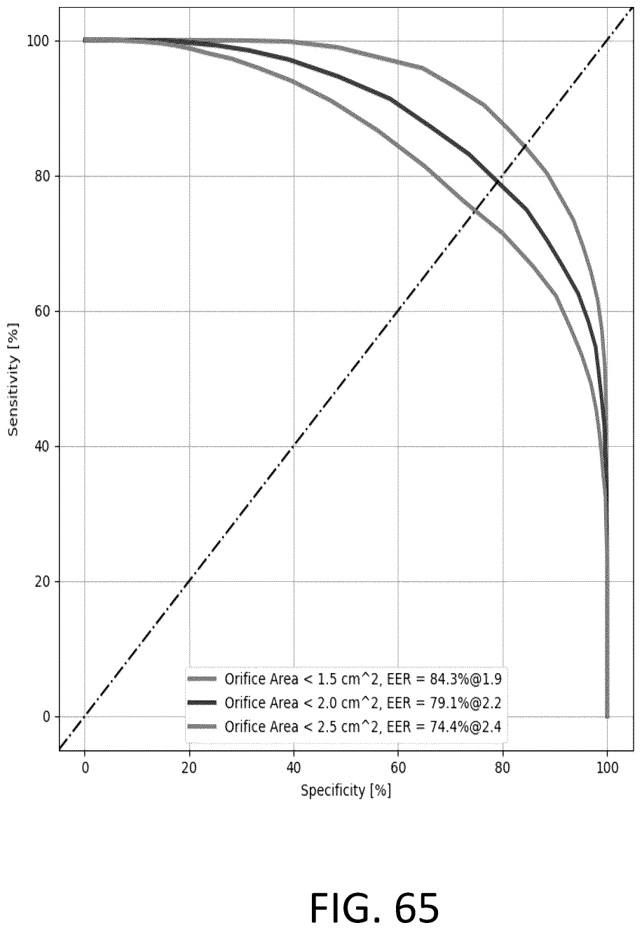

FIG. 65 is a plot of the true positive rate (Sensitivity) against the false positive rate (Specificity) for different threshold values. Sensitivity represents the proportion of true positive predictions correctly identified by the model while Specificity indicates the proportion of true negative predictions correctly identified. FIG. 65 is the Sensitivity versus Specificity for the volume predictions at the carotid artery with age added as supplemental data with 10% representative noise included. The equal error rate (EER) at an aortic valve area of 1.5 cm² is 84.3%. For aortic valve areas of 2.0 cm² and 2.5 cm², the EER are 79.1% and 74.4% respectively.

### Auscultation Comparison

The invention represents a significant advancement in aortic stenosis screening as the measurement is not dependent on the presence of turbulence and the propagation of those sound waves through the thorax. Laminar flow occurs when blood moves in ordered, parallel layers through the vasculature with no obstructions to agitate the layers. An example would be an easy-flowing, straight river with a smooth, even bottom and shoreline. The middle of the river may move faster than the banks, but all the water moves forward in straight paths.

Turbulent flow describes a situation where the flow pathway becomes disorganized, layers break formation, and eddy currents are formed. Picture a river that is rapid and winding with variable depths and many obstructions that cause eddies, white water, and other ‘rough water’ features. In arteries, turbulent blood flow can occur due to narrowing, branching or bends. The pronounced bend in the ascending aorta increases the possibility of turbulent flow. Turbulent flow generates sound, creating murmurs, carotid bruits, and other audible diagnostic vibrations.

Reynolds number is an equation that calculates laminar versus turbulent flow. It is expressed as follows:

Reynolds number=[diameter×velocity×density]/viscosity

Laminar flow occurs at low Reynolds numbers where viscous forces are dominant, and it is characterized by smooth, constant fluid motion; turbulent flow occurs at high Reynolds numbers and is dominated by inertial forces, which tend to produce chaotic eddies, vortices, and other flow instabilities.

The following discussion seeks to communicate the relationship between Reynolds number and turbulent flow and the relationship between turbulent flow and an auditory murmur. The analysis demonstrates the impact of thorax wall vibration transmission and the inherent benefits of a flow-based measurement approach. Published literature of invasive clinical studies and computational fluid dynamics simulations define a broad range of Reynolds numbers that create turbulent flow in the aorta. In vivo studies have reported turbulent flow in the ascending aorta of AVS subjects with peak Reynolds numbers ranging from 5,700 to 8,900, (Stein, Paul D., and Hani N. Sabbah. “Turbulent blood flow in the ascending aorta of humans with normal and diseased aortic valves.” Circulation research 39.1 (1976): 58-65.) Computational fluid dynamics simulations by Caballero et al. demonstrate that “disturbed flow was present in the aorta when the peak Reynolds number was on the order of 5000-6000. (Caballero, Andres D., and S. J. C. E. Laín. “A review on computational fluid dynamics modeling in human thoracic aorta.” Cardiovascular Engineering and Technology 4 (2013): 103-130.)

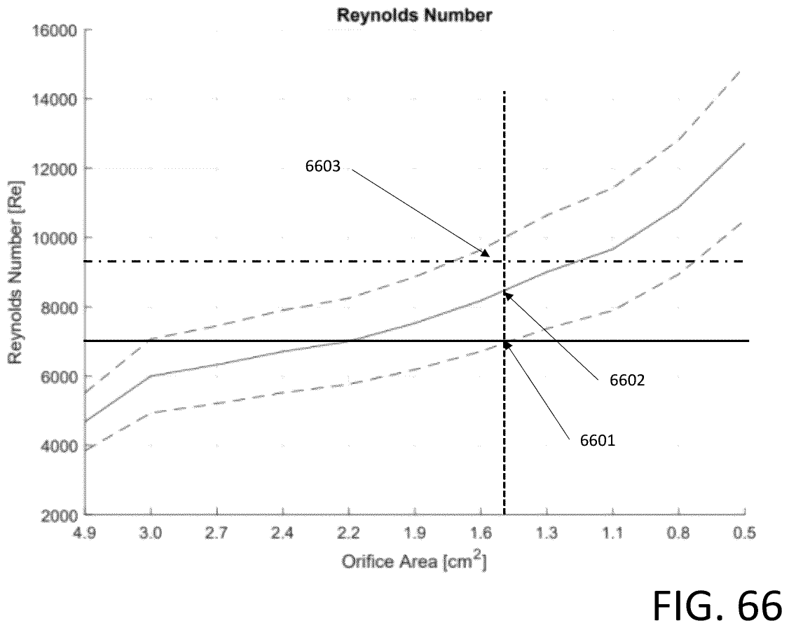

For explanation purposes, assume a Reynolds number of 7000 (average of the range noted) results in turbulence flow and creates a murmur. For evaluation, an aortic valve area of 1.5 cm² was selected as it is often referred to as mild aortic stenosis and is an area where clinical symptoms often begin. FIG. 66 illustrates that approximately 84% of patients would have an ejection murmur and should be screened effectively, assuming no vibration loss through the thorax, (6601).

The second step in this comparison is to evaluate clinical screening performance at an aortic valve area of 1.5 cm². The sensitivity of cardiac auscultation for detecting mild aortic stenosis is 32% (Gardezi et al., “Cardiac auscultation poorly predicts the presence of valvular heart disease in asymptomatic primary care patients. Heart. 2018). Using the Reynolds number versus orifice area plot of FIG. 66, the mean Reynolds number calculated at an orifice of 1.5 cm² is 8200 with a standard deviation of 1500, (6602). Using the Z score, a Reynolds number threshold for detection by auscultation with a sensitivity of 32% at an aortic valve area of 1.5 cm² can be calculated as 9,065 (6603). This analysis connects the observed level of auscultation sensitivity with a degree of turbulence defined by a Reynolds number of 9,065. Thus, to obtain a 32% sensitivity of auscultation, the analysis shows that a Reynolds number of 9,065 must be obtained.

With an awareness that the sensitivity numbers are different (84% vs 32%), the comparison demonstrates a significant impact of thorax sound transmission necessitating a higher degree of turbulence for creating an audible murmur (7,000 versus 9,065). In summary, a Reynolds number of 7000 creates a murmur supporting a sensitivity of 84% if no transmission through the thorax is needed.

However, in clinical practice, thorax sound propagation attenuates the vibration such that a Reynolds number of 9,065 creates an audible murmur with a sensitivity of 32%. The Reynolds number needed to create a sensitivity of 84% would be significantly higher.

The detrimental influence of thorax sound propagation was demonstrated by Gardezi et al. via a reduction in sensitivity of 50% in patients with Body Mass Index (BMI)≥25 kg/m², (“Cardiac auscultation poorly predicts the presence of valvular heart disease in asymptomatic primary care patients. Heart. 2018). The increased BMI creates a longer path between murmur generation and murmur detection. The result is a greater degree of murmur attenuation with a direct negative impact on the sensitivity of auscultation for aortic valve stenosis.

In stark contrast, the in silico simulations at an aortic valve area of 1.5 cm² demonstrated a sensitivity of 91% at an equal error rate, FIG. 63. The invention has less sensitivity to variance in BMI as it does not depend on sound wave propagation through the thorax.

What is claimed is:

1. A system for determining a quantitative assessment of aortic stenosis in a patient, comprising:
(a) a speckle plethysmograph configured to noninvasively measure blood flow at a peripheral sampling location of the patient;
(b) a data acquisition system for collecting temporal variations of a speckle pattern measured by the speckle plethysmograph;
(c) a programmed data processor executing data analysis software configured to produce a speckle plethysmogram from the temporal variations; and
(d) a left ventricular outflow tract assessment system configured to produce a quantitative assessment of the aortic valve area from the speckle plethysmogram.

2. A system for quantifying a degree of aortic stenosis in a patient, comprising:
(a) a coherent light source configured to illuminate a target area of the patient's skin overlying a peripheral sampling location with coherent light to generate a speckle pattern;
(b) an imaging device configured to capture temporal variations of the speckle pattern related to the cardiac cycle;
(c) a sensor control system configured to operate the coherent light source and the imaging device during a measurement period comprising at least one cardiac cycle to produce a measurement signal during the systolic and diastolic phases of the at least one cardiac cycle;
(d) a data acquisition system configured to acquire temporal variations of the speckle pattern over at least one cardiac cycle and produce a speckle plethysmogram from the temporal variations of the speckle pattern;
(f) a left ventricular outflow tract assessment system comprising a programmed data processor programmed to quantify the degree of aortic stenosis from the speckle plethysmogram;
(g) an output device configured to provide a quantitative output indicative of the degree of aortic stenosis.

3. The system of claim 2, further comprising;
(h) a physiological assessment system configured to determine the presence of a cardiac vagal control from the temporal variations of the speckle pattern based on (h1) an interbeat time interval between successive openings of the patient's aortic valve from each of two or more cardiac cycles, and (h2) a variability between two or more interbeat time intervals; and wherein the output device is further configured to provide an indication of the presence of cardiac vagal control.

4. The system of claim 2, wherein the programmed data processor is programmed to use a set of parameters defining a mapping function between the measured speckle plethysmogram and the area of the aortic valve and to determine the severity of aortic stenosis.

5. The system of claim 2, wherein the programmed data processor is programmed to a prediction model where the prediction model comprises multiple hierarchical layers.

6. A method for detecting and quantifying aortic stenosis in a patient independent of an audible murmur, the method comprising;
(a) illuminating a target area of the patient's skin overlying a peripheral sampling location with coherent light to generate a speckle pattern;
(b) capturing temporal variations of the speckle pattern with an imaging device, wherein the temporal variations correspond to physiological changes related to the cardiac cycle;
(c) determining the presence of aortic stenosis in the patient from temporal variations in the speckle pattern.

7. The method of claim 6, wherein step (c) comprises processing the temporal variations using data analysis software configured to produce a speckle plethysmogram from the temporal variations, wherein the speckle plethysmogram includes blood flow characteristics indicative of aortic valve functionality.

8. The method of claim 7 further comprising;
(d) analyzing the measured speckle plethysmogram to identify flow characteristics, wherein the analysis includes assessing for peripheral flow alterations due to aortic stenosis in the speckle plethysmogram;
(e) determining a quantitative indication of the degree of aortic stenosis in the patient from the peripheral flow alterations.

9. The method of claim 8 further comprising using a left ventricular outflow tract assessment system to produce a quantitative assessment of aortic valve area from the speckle plethysmogram.

10. A method of quantifying the degree of early-stage aortic stenosis, independent of an audible murmur in a patient, comprising:
(a) noninvasively measuring blood flow at a peripheral sampling location of the patient using a speckle plethysmograph for at least one heart cycle to obtain a speckle plethysmogram;
(b) analyzing the measured speckle plethysmogram to identify flow characteristics, wherein the analysis includes assessing for peripheral flow alterations due to aortic stenosis in the speckle plethysmogram;
(c) determining a quantitative indication of the degree of aortic stenosis in the patient from the peripheral flow alterations.

11. The method of claim 10, wherein step (a) further comprises positioning a patient in a supine posture to induce preload independence during step (a).

12. The method of claim 10, further comprising:
(d) acquiring a measurement signal by noninvasively detecting changes in blood volume or flow in a measurement region of the patient, where the changes are indicative of opening of the patient's aortic valve;
(e) determining from the measurement signal an interbeat interval from an aortic valve opening until a successive aortic valve opening,
(f) determining a presence of preload independence control based on the one or more interbeat intervals; and
(g) determining that the quantitative indication of the degree of aortic stenosis in step (c) is valid if preload independence is present.

13. The method of claim 11, further comprising:
(d) acquiring a measurement signal by noninvasively detecting changes in blood volume or flow in a measurement region of the patient, where the changes are indicative of opening and closing of the patient's aortic valve;
(e) determining from the measurement signal an ejection time from an aortic valve opening until a successive aortic valve closing, and two or more interbeat intervals, where the interbeat interval is the time from an aortic valve opening until a successive aortic valve opening;
(f) determining a presence of preload independence based on the two or more interbeat intervals and one or more ejection times;
(g) determining that the quantitative indication of the degree of aortic stenosis in step (c) is valid if preload independence is present.

14. The method of claim 10 further comprises determining preload independence by conducting a venous return change evaluation and analyzing resulting data with a physiological assessment system and determining that the quantitative indication of the degree of aortic stenosis in step (c) is valid if preload independence is present.

15. The method of claim 14 where the venous return change evaluation comprises changing the body position of the patient to create a change in venous return, and the physiological assessment system determines preload independence from interbeat interval or ejection time or a combination thereof.

16. The method of claim 10, further comprising:
(d) acquiring a measurement signal by noninvasively detecting changes in blood volume or flow in a measurement region of the patient, where the changes are indicative of opening of the patient's aortic valve;
(e) determining from the measurement signal and two or more interbeat intervals, where the interbeat interval is the time from an aortic valve opening until a successive aortic valve opening;
(f) determining a presence of cardiac vagal control based on a measure of variability of the two or more interbeat intervals; and
(g) determining that the quantitative indication of the degree of aortic stenosis in step c is valid if cardiac vagal is present.

17. The method of claim 10, further comprising:
(d) acquiring a measurement signal by noninvasively detecting changes in blood volume or flow in a measurement region of the patient, where the changes are indicative of opening and closing of the patient's aortic valve;
(e) determining from the measurement signal an ejection time from an aortic valve opening until a successive aortic valve closing, and two or more interbeat intervals, where the interbeat interval is the time from an aortic valve opening until a successive aortic valve opening;
(f) determining a presence of cardiac vagal control based on a measure of the variability of the two or more ejection times and a measure of the variability of the two or more interbeat intervals; and
(g) determining that the quantitative indication of the degree of aortic stenosis in step c is valid if cardiac vagal control is present.

18. The method of claim 10, further comprising;
(d) providing a noninvasive sensor system, comprising one or more cardiovascular sensors configured to produce a signal that indicates a time of opening and closing of the user's aortic valve;
(e) providing a sensor control system configured to operate the noninvasive sensor system and data acquisition system to record a measurement signal that indicates the times of opening and closing of the user's aortic valve during two or more successive cardiac cycles;
(f) providing a physiological assessment system configured to determine the presence of a repeatable cardiac state from the measurement signal based on (f1) an interbeat time interval between successive openings of the user's aortic valve from each of two or more cardiac cycles, and (f2) a variability between two or more interbeat time intervals; and
wherein determining a quantitative indication of the degree of aortic stenosis comprises determining a presence of a repeatable cardiac state from steps (d), (e), and (f), and wherein the quantitative indication of the degree of aortic stenosis is qualified by the determination of a repeatable cardiac state.
